SEQUENCE LISTING (12 sequences)

SEQ ID NO: 1 - protein, 10 aa, synthetic construct
DVWHFSLVDK

SEQ ID NO: 2 - protein, 10 aa, synthetic construct
DFWNIGMVHK

SEQ ID NO: 3 - protein, 10 aa, synthetic construct
DVGEISAIDK

SEQ ID NO: 4 - protein, 10 aa, synthetic construct
DLTKLGHIGK

SEQ ID NO: 5 - protein, 10 aa, synthetic construct
DILQLGVIWK

SEQ ID NO: 6 - protein, 10 aa, synthetic construct
DLTKIGEVGK

SEQ ID NO: 7 - protein, 10 aa, synthetic construct
DLTKLGHIGH

SEQ ID NO: 8 - protein, 10 aa, synthetic construct
DILQLGVIWK

SEQ ID NO: 9 - protein, 10 aa, synthetic construct
DGTITVRVDK

SEQ ID NO: 10 - protein, 10 aa, synthetic construct
DAFWVGVTFK

SEQ ID NO: 11 - protein, 10 aa, synthetic construct
DVGEISAIDK

SEQ ID NO: 12 - protein, 10 aa, synthetic construct
DAWTIAAICK

(19) United States
(12) Patent Application Publication
(10) Pub. No.: US 2026/0193295 A1
(43) Pub. Date: Jul. 9, 2026

(54) CILAGICIN COMPOUNDS AND METHODS OF USE THEREOF

(71) Applicant: The Rockefeller University, New York, NY (US)

(72) Inventors: Sean Brady, New York, NY (US); Zongqiang Wang, New York, NY (US); Bimal Koirala, New York, NY (US)

(21) Appl. No.: 18/868,437

(22) PCT Filed: May 25, 2023
(86) PCT No.: PCT/US2023/067459

§ 371 Date: Nov. 22, 2024

Related U.S. Application Data
(60) Provisional application No. 63/345,699, filed on May 25, 2022.

(51) Int. Cl. C07K 11/02; A61K 38/00; A61P 31/04

(52) U.S. Cl. CPC C07K 11/02; A61P 31/04; A61K 38/00

(57) ABSTRACT

The present invention provides methods, compositions, and articles of manufacture useful for treatment of multidrug-resistant pathogens and related conditions. The present invention provides compositions and methods incorporating and utilizing cilagicin compounds or derivatives or variants thereof.

### CROSS-REFERENCE TO RELATED APPLICATIONS

This application claims priority to U.S. Provisional Application Ser. No. 63/345,699, filed May 25, 2022, the disclosure of which is hereby incorporated by reference herein in its entirety.

### STATEMENT OF GOVERNMENT FUNDING

This invention was made with government support under 1U19AI142731 and 5R35GM122559 awarded by the National Institutes of Health. The government has certain rights in the invention.

### BACKGROUND OF THE INVENTION

The discovery and therapeutic development of natural product antibiotics, especially those produced by microbes, has dramatically reduced mortality caused by bacterial infections (Rossiter et al., 2017, Chemical reviews, 117:12415-12474). Unfortunately, this situation is challenged by the fact that antibiotic resistance is rising at a faster rate than the introduction of molecules with modes of action (MOAs) capable of circumventing existing resistance mechanisms (Davies et al., 2010, Microbiol Mol Biol Rev, 74:417-433; Aslam et al., 2018, Infect Drug Resist, 11:1645-1658).

From a clinical development standpoint, non-ribosomal peptide synthetase (NRPS) encoded lipopeptides are an appealing potential source of future antibiotics as they have a history of inhibiting bacterial growth by unique and diverse MOAs (Hamley et al., 2015, Chem Commun (Camb), 51:8574-8583; Raaijmakers et al., 2010, FEMS Microbiol Rev, 34:1037-1062). Bacterial genome sequencing efforts have uncovered a large number of biosynthetic gene clusters (BGCs) that do not appear to encode for known natural products, including many BGCs that are predicted to encode undescribed lipopeptides. These BGCs likely contain genetic instructions for the biosynthesis of antibiotics with diverse MOAs that could help to replenish antibiotic discovery pipelines. Unfortunately, the vast majority of sequenced BGCs remain silent in the laboratory and therefore, the molecules they encode remain a mystery.

Thus, there is a need in the art for new compositions and methods for treating infections. The present invention satisfies this unmet need in the art.

### SUMMARY OF THE INVENTION

In one aspect, the present invention provides a compound comprising the amino acid sequence D(X^{A})ₐK.

In some embodiments, the compound is a zwitterion. In some embodiments, the zwitterion comprises at least two positively charged residues and at least two negatively charged residues.

In some embodiments, the compound is a cyclic compound.

In some embodiments, the compound is a linear compound.

In some embodiments, a is an integer from 8 to 100. In one embodiment, a is an integer represented by 8.

In some embodiments, each occurrence of X^{A} is independently selected from a natural amino acid, functionalized natural amino acid, unnatural amino acid, functionalized unnatural amino acid, or any combination thereof. In some embodiments, the functionalized natural amino acid or the functionalized unnatural amino acid comprises a functional group selected from the group consisting of at least one selected from FIG. 4A, at least one selected from FIG. 6, at least one selected from FIG. 7, or any combination thereof.

In some embodiments, the amino acid sequence D(X^{A})ₐK comprises at least one amino acid sequence selected from at least one amino acid sequence as set forth in SEQ ID NOs: 1-12 or a fragment thereof.

In some embodiments, the compound is a compound having the structure of Formula (I)

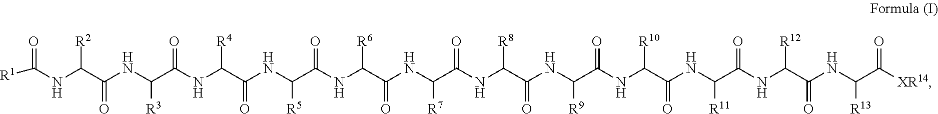

or a racemate, an enantiomer, a diastereomer, a pharmaceutically acceptable salt, or a derivative thereof.

In some embodiments, X is selected from O, S, or N(R¹⁴).

In some embodiments, each occurrence of R¹, R², R³, R⁴, R⁵, R⁶, R⁷, R⁸, R⁹, R¹⁰, R¹¹, R¹², R¹³, and R¹⁴ is independently selected from hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, or any combinations thereof.

In some embodiments, R² and X are optionally fused or joined to form a ring.

In some embodiments, R³ and X are optionally fused or joined to form a ring.

In some embodiments, R⁴ and X are optionally fused or joined to form a ring.

In some embodiments, the compound having the structure of Formula (I) is a compound having the structure selected from the group consisting of

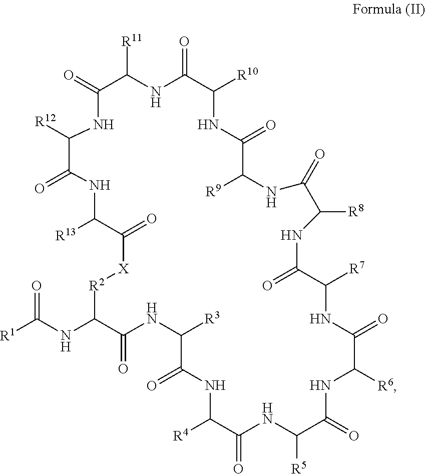

or a racemate, an enantiomer, a diastereomer, a pharmaceutically acceptable salt, or a derivative thereof;

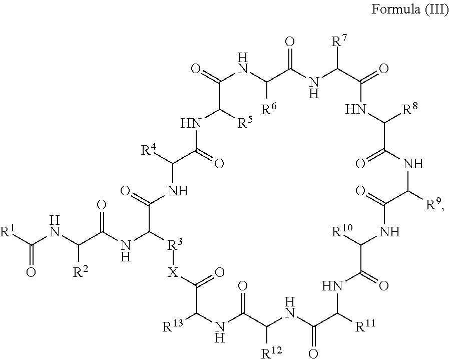

or a racemate, an enantiomer, a diastereomer, a pharmaceutically acceptable salt, or a derivative thereof; and

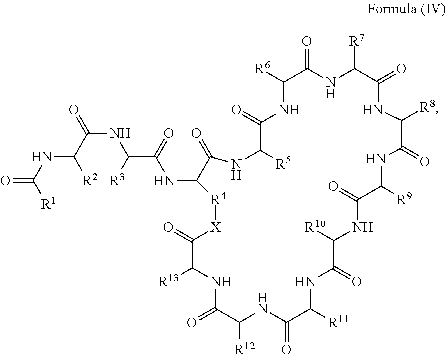

or a racemate, an enantiomer, a diastereomer, a pharmaceutically acceptable salt, or a derivative thereof.

In some embodiments, the compound is a compound selected from:

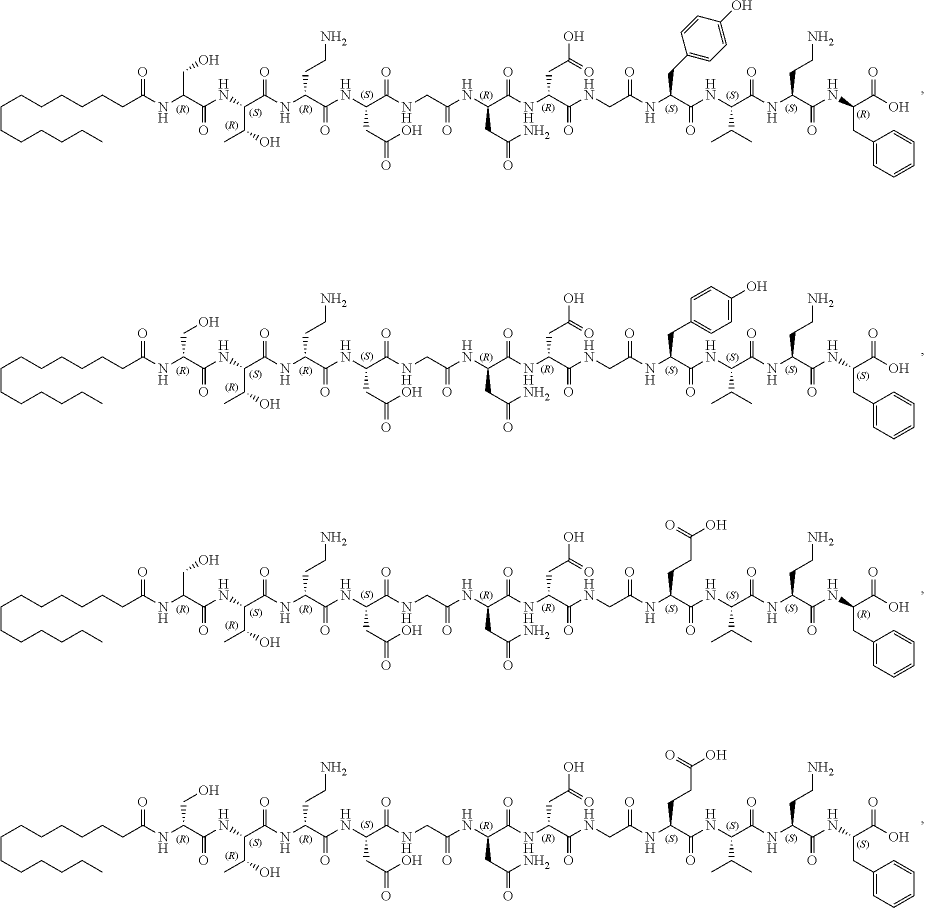

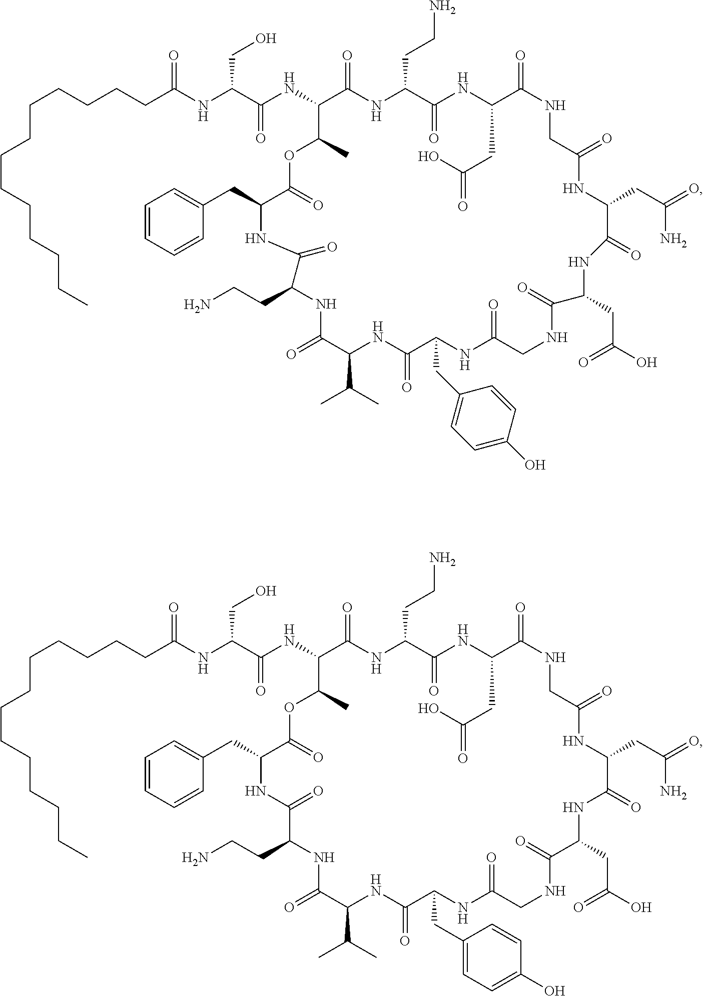 [SMILES: CCCCCCCCCCCCCC(=O)N[C@H](CO)C(=O)N[C@@H]1C(=O)N[C@H](CCN)C(=O)N[C@@H](CC(=O)O)C(=O)NCC(=O)N[C@H](CC(N)=O)C(=O)N[C@H](CC(=O)O)C(=O)NCC(=O)N[C@@H](Cc2ccc(O)cc2)C(=O)N[C@@H](C(C)C)C(=O)N[C@@H](CCN)C(=O)N[C@@H](Cc2ccccc2)C(=O)O[C@@H]1C ; CCCCCCCCCCCCCC(=O)N[C@H](CO)C(=O)N[C@@H]1C(=O)N[C@H](CCN)C(=O)N[C@@H](CC(=O)O)C(=O)NCC(=O)N[C@H](CC(N)=O)C(=O)N[C@H](CC(=O)O)C(=O)NCC(=O)N[C@@H](Cc2ccc(O)cc2)C(=O)N[C@@H](C(C)C)C(=O)N[C@@H](CCN)C(=O)N[C@H](Cc2ccccc2)C(=O)O[C@@H]1C]

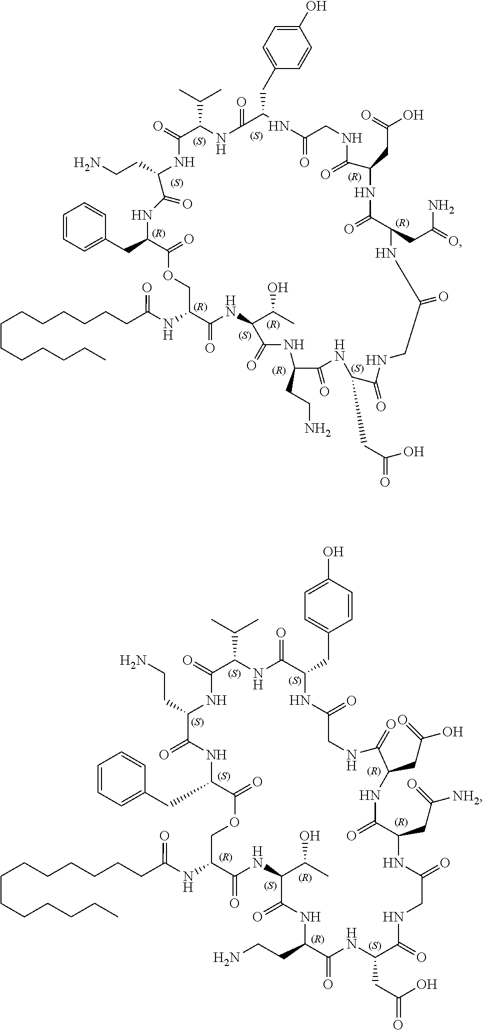

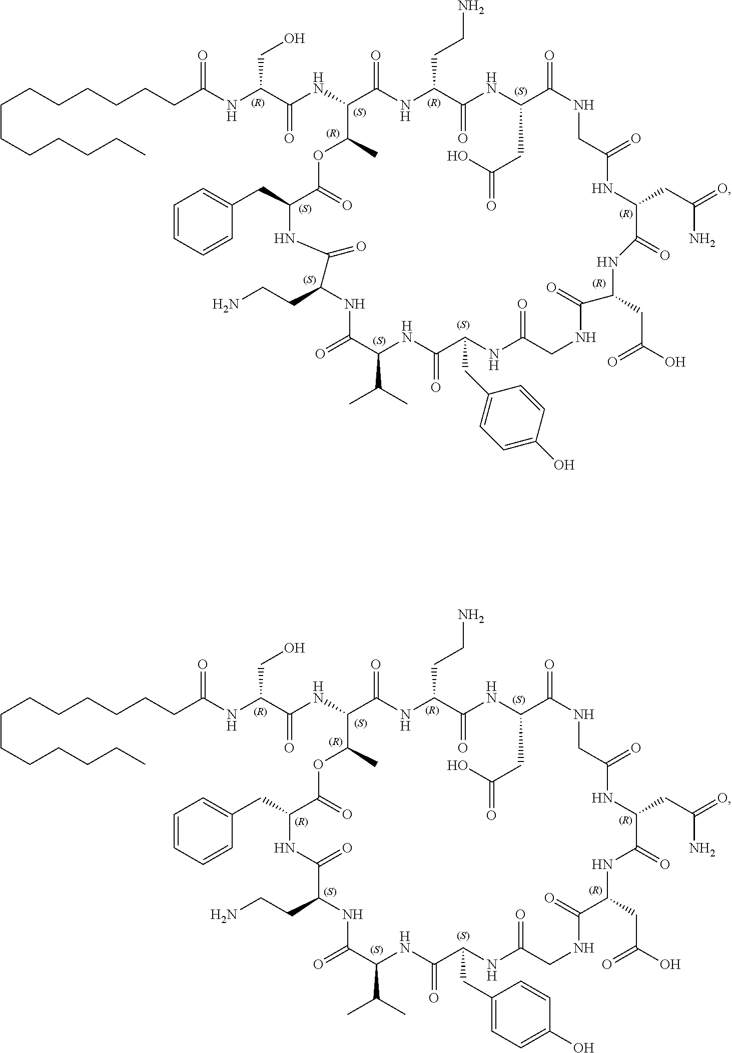

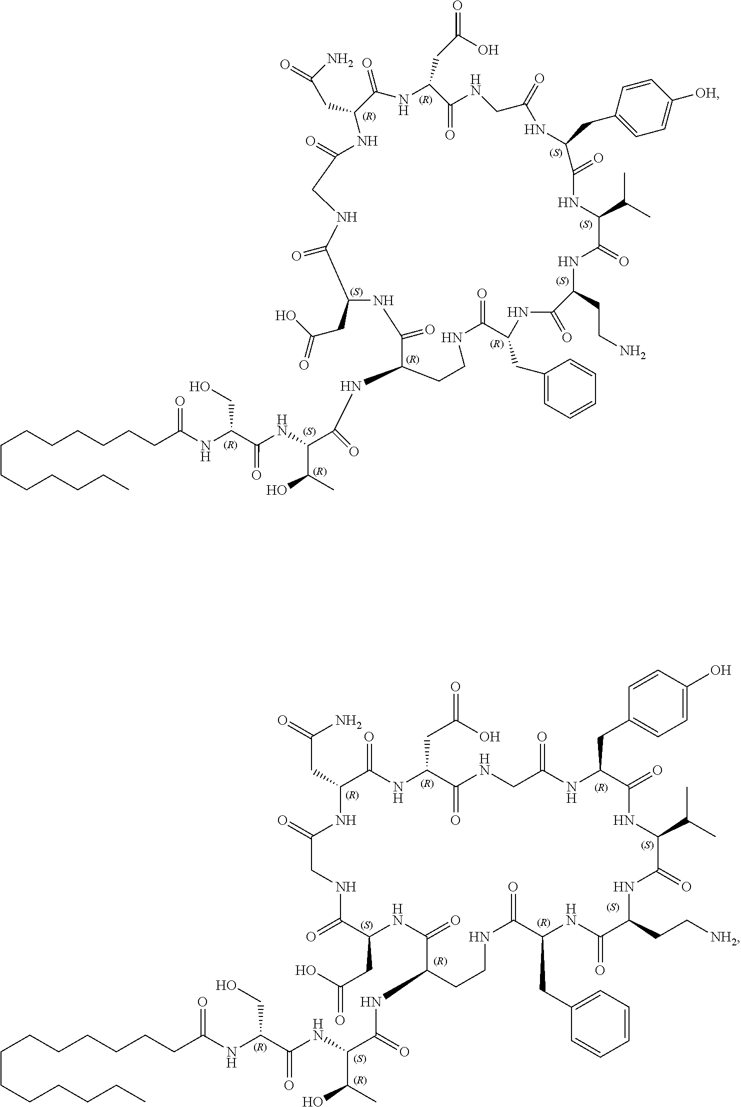

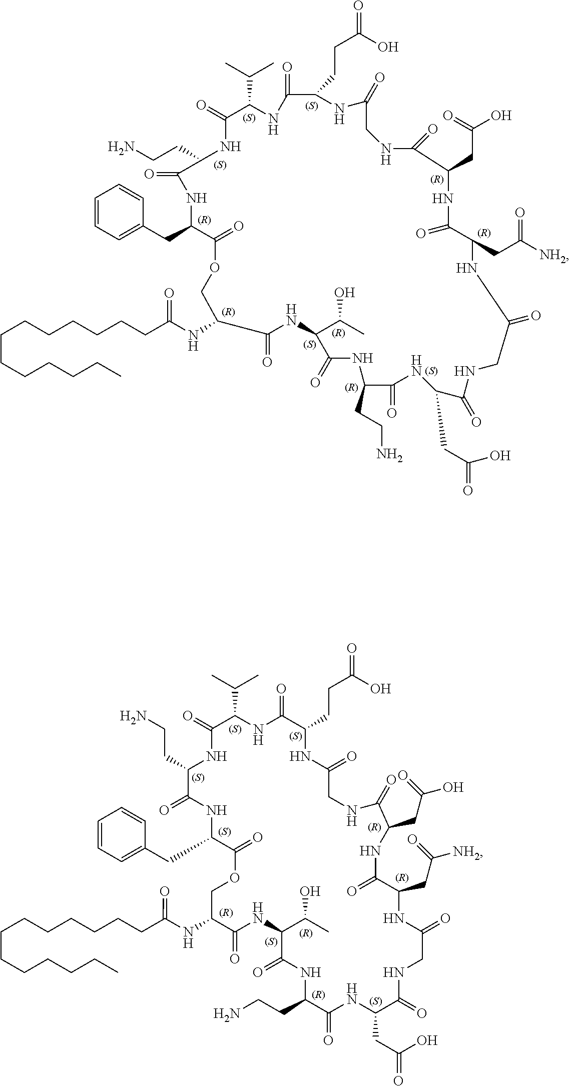

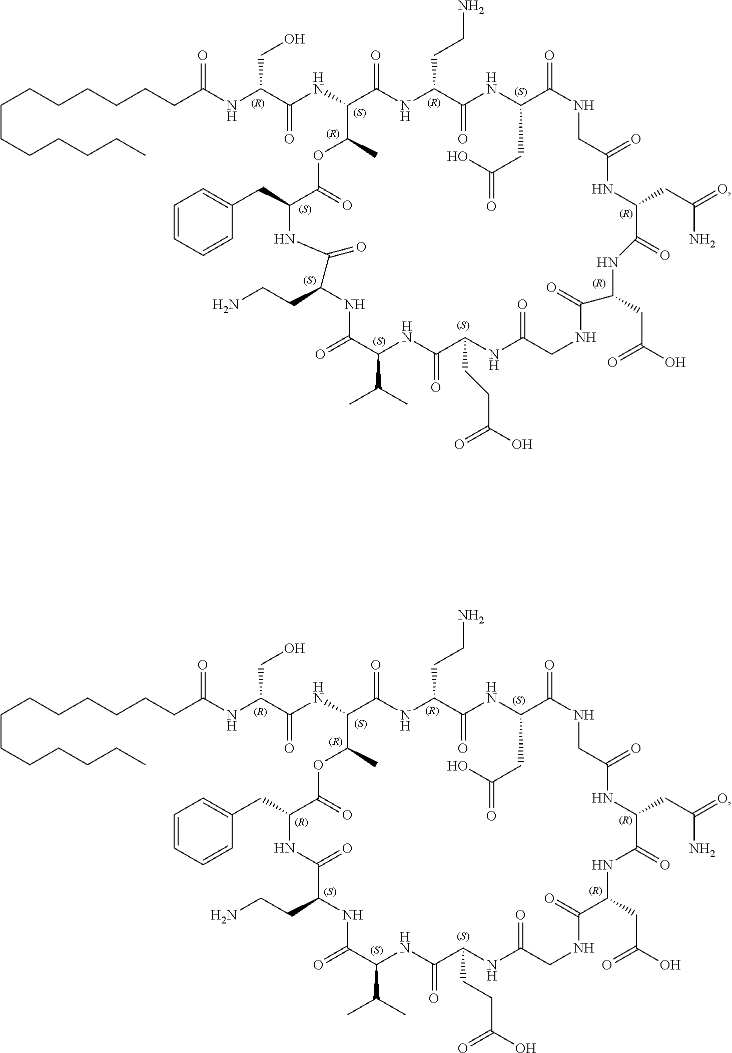

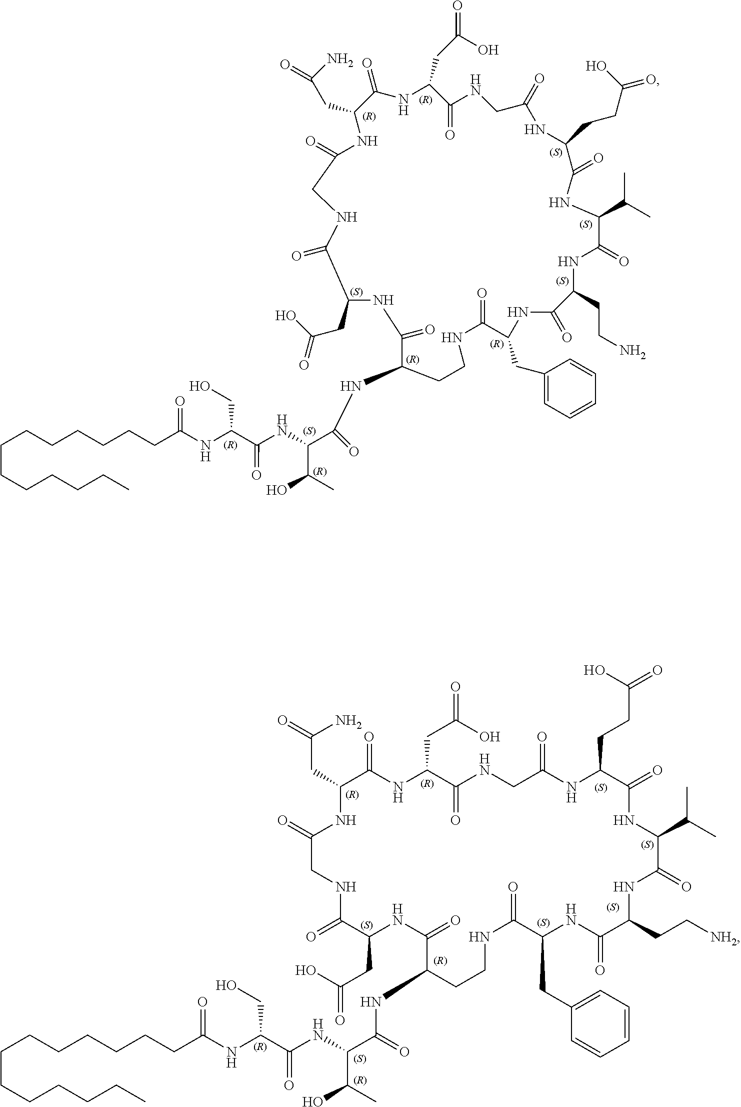

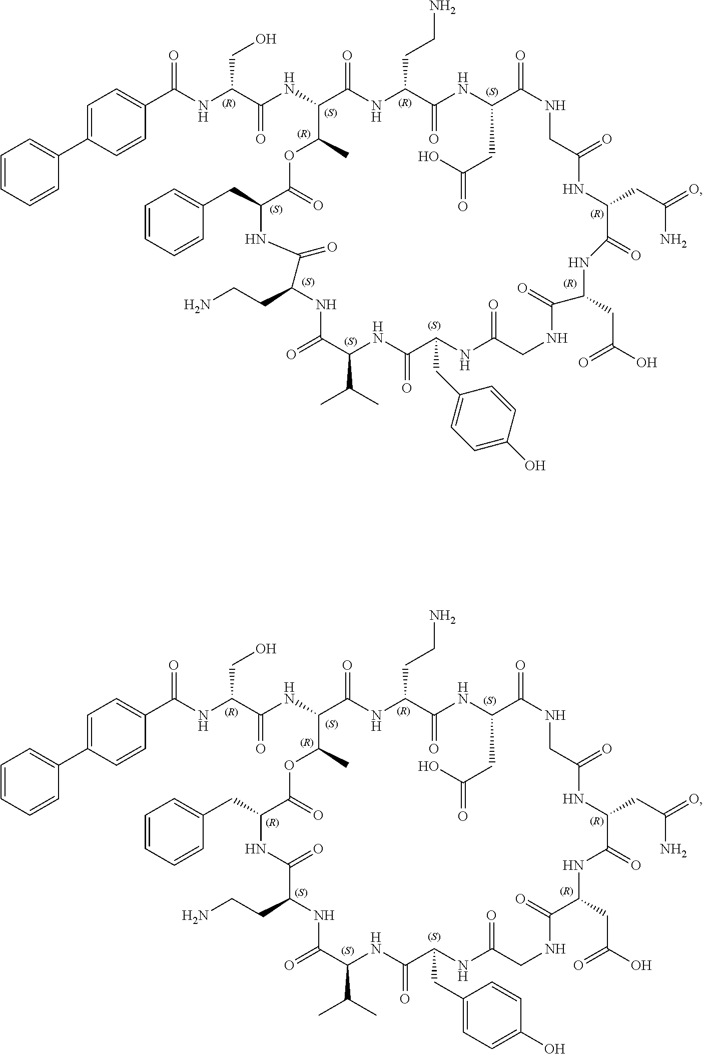

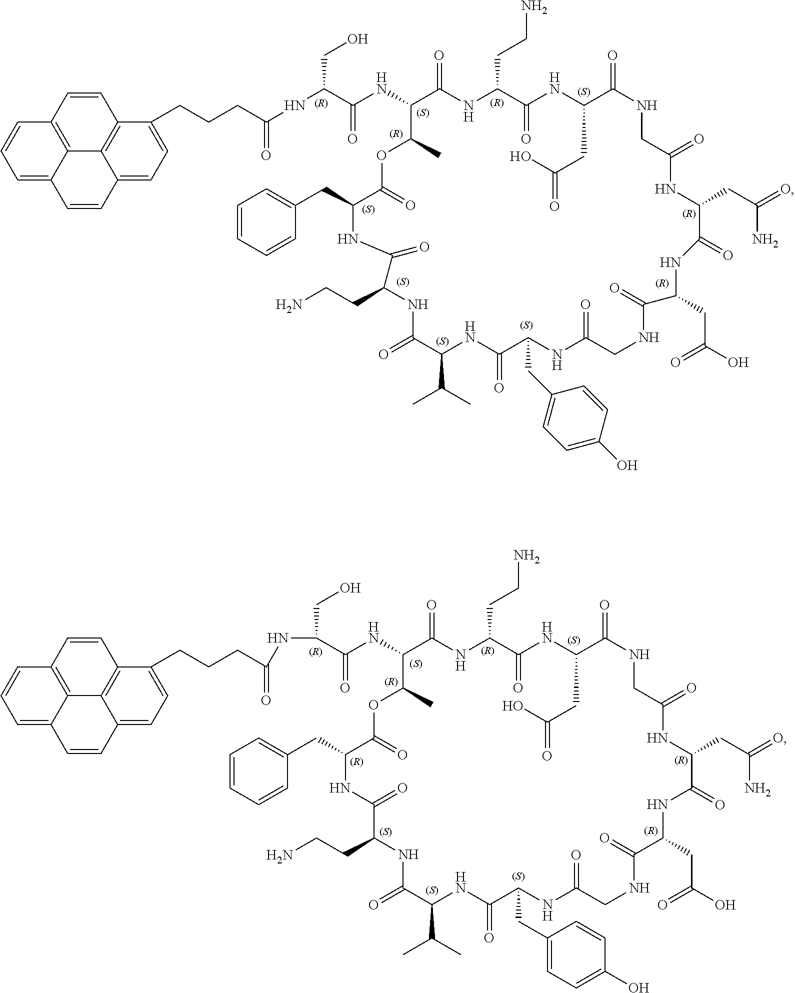

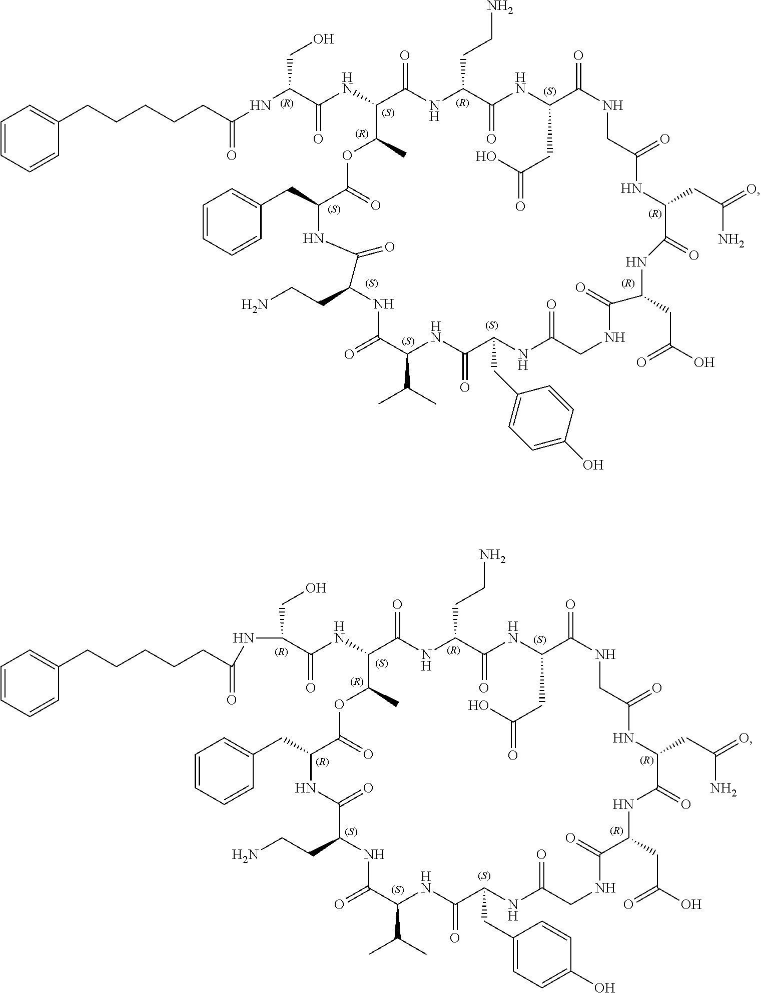

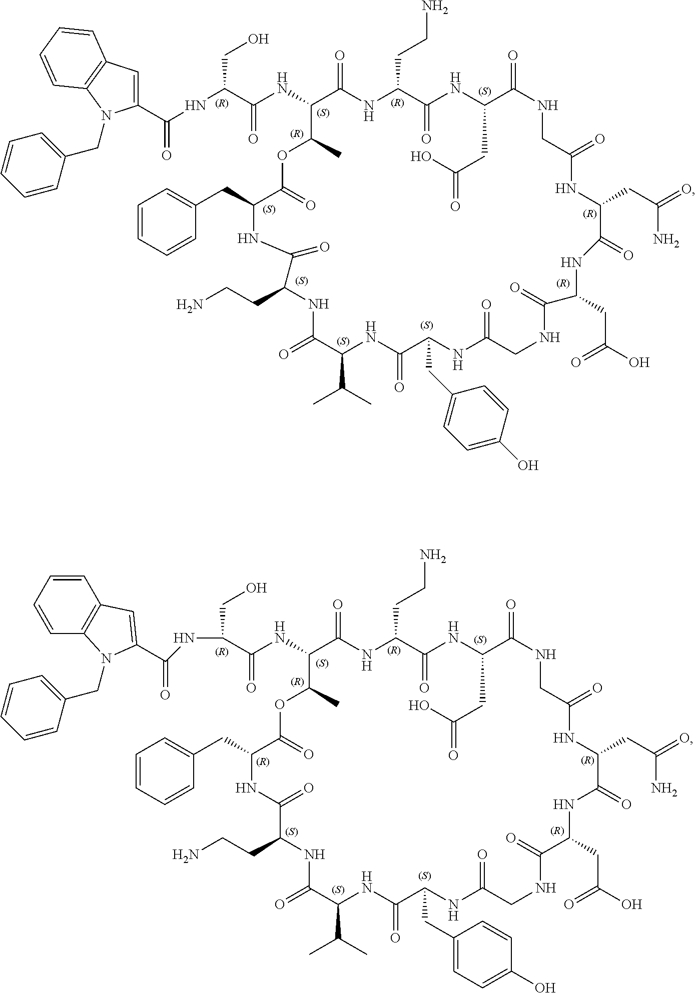

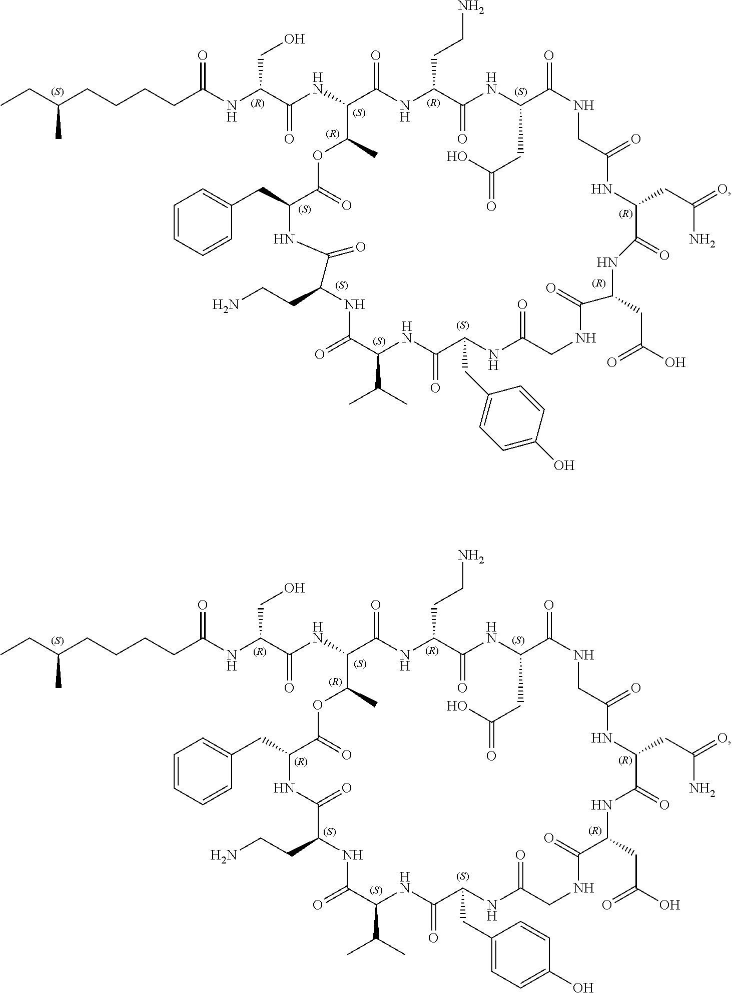

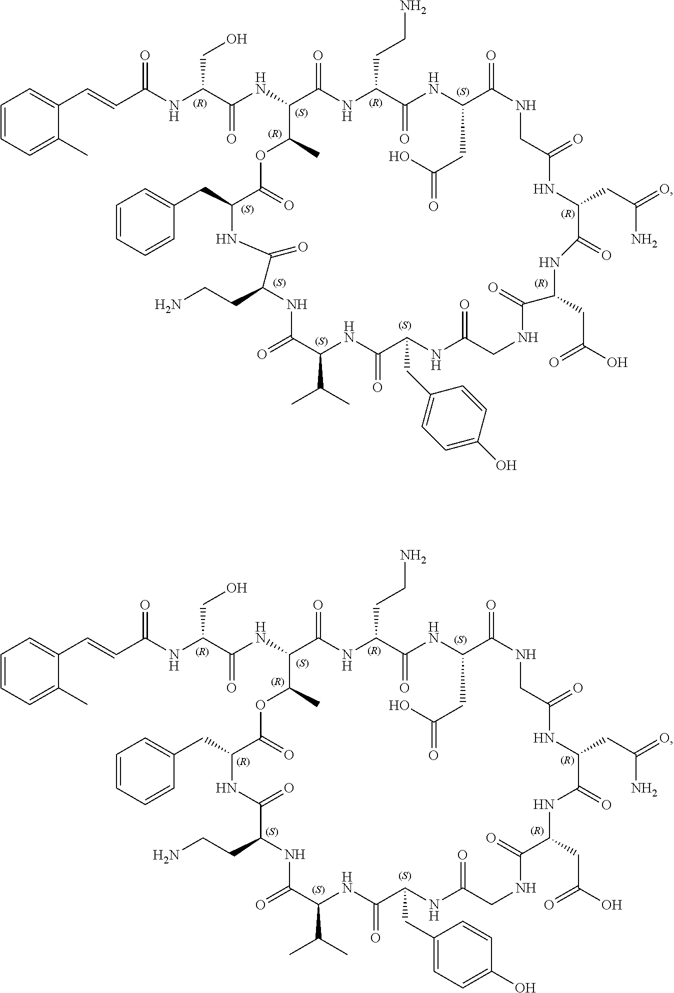

or a racemate, an enantiomer, a diastereomer, a pharmaceutically acceptable salt, or a derivative thereof.

In some embodiments, the compound inhibits cell wall biosynthesis.

In some embodiments, the compound specifically binds at least one undecaprenyl phosphorylate.

In various aspects, the present invention also provides a pharmaceutical composition comprising at least one compound of the present invention or a racemate, an enantiomer, a diastereomer, a pharmaceutically acceptable salt, or a derivative thereof.

In other aspects, the present invention provides an isolated nucleic acid molecule encoding at least one compound of the present invention or a racemate, an enantiomer, a diastereomer, a pharmaceutically acceptable salt, or a derivative thereof.

In some embodiments, the nucleic acid molecule comprises at least one nucleotide sequence of FIG. 1.

In other aspects, the present invention provides a genetically engineered cell producing at least one compound of the present invention or a racemate, an enantiomer, a diastereomer, a pharmaceutically acceptable salt, or a derivative thereof.

In other aspects, the present invention provides a method of treating or preventing a bacterial infection in a subject in need thereof.

In some embodiments, the method comprises administering at least one compound of the present invention or a composition thereof to the subject.

In some embodiments, the subject is exposed to or infected with a pathogen.

In some embodiments, the pathogen is bacteria. In some embodiments, the bacteria is selected from drug resistant bacteria, gram positive bacteria, or any combination thereof. In some embodiments, the bacteria is selected from Bacillus subtilis, Clostridium difficile, Enterococcus faecium, Enterococcus gallinarum, Enterococcus casseliflavus, Escherichia coli, Streptococcus agalactiae, Streptococcus pneumoniae, Streptococcus pyrogens, Staphylococcus aureus, Klebsiella pneumoniae, Acinetobacter baumannii, Pseudomonas aeruginosa, Enterobacter cloacae, Enterobacter species, or any combination thereof.

In some embodiments, the method further comprises administering a second therapeutic. In some embodiments, the second therapeutic is an antibiotic.

In other aspects, the present invention provides a method of inhibiting the growth of or killing a bacterial cell.

In some embodiments, the method comprises contacting the bacterial cell with at least one compound of the present invention or a composition thereof.

In other aspects, the present invention provides a method of biosynthesizing at least one compound of the present invention.

In some embodiments, the method comprises a) providing a nucleic acid to a host or a growth medium, wherein the nucleic acid encodes the amino acid sequence D(X^{A})ₐK or a fragment thereof, b) incubating the host in a growth medium; and c) isolating the compound from the host or the growth medium.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1, comprising FIG. 1A through FIG. 1E, depicts a schematic representation of the discovery of cilagicin. FIG. 1A depicts representative condensation starter domains (Cs) from sequenced NRPS BGCs were used to construct a phylogenetic tree. Clades associated with characterized antibiotic BGCs are labeled. The “orphan” cilagicin clade is labeled in blue. FIG. 1B depicts representative cil BGC containing three NRPS open reading frames (cilC-E). Biosynthesis of cilagicin is to start from a Cs domain in CilC. It then proceeds using one A (adenylation) and T (thiolation) domain containing initiation module followed by 11 C (condensation), A and T domain containing extender modules. The substrate specificity of the cil BGC A-domains was determined based on a comparison of each A-domain's substrate binding pocket to the 10 amino acid A-domain signature sequences found in functional characterized BGCs. E (epimerization) domains in modules 1, 3, 6 and 7 result in the incorporation of D amino acids. The TE (thioesterase) domain at the end of CilE releases the mature structure from the final T domain as either a linear or cyclic product. FIG. 1C depicts a schematic representation of the four different peptide topologies that were synthesized from the linear peptide generated to arise from the cil BGC. Position nine was either Tyr (a) or Glu (b). L is a linear peptide. C1, C2 and C3 are cyclized through the C-terminal carboxyl group and Ser-1, Thr-2 or Dab-3, respectively. Dab, 2, 4-diaminobutyric acid. FIG. 1D depicts representation MIC data against the ESKAPE pathogens for the eight synthetic structures depicted in FIG. 1C. Concentrations tested ranged from 1 μg/mL (blue) to 64 μg/mL (white). Data are representative of 3 independent experiments. FIG. 1E depicts a schematic representation of a structure of the antibiotic cilagicin, which corresponds to C2a in FIG. 1C.

FIG. 2, comprising FIG. 2A through FIG. 2F, depicts representative results demonstrating the cilagicin mode of action. FIG. 2A depicts representative results demonstrating the survival of S. aureus USA300 after timed exposure to 10× the MIC of cilagicin. DMSO and Vancomycin (10×MIC) were included as controls. CFU were counted three independent times and plotted as mean±SD. FIG. 2B depicts representative scanning electron microscopy image of S. aureus USA300 cultures treated with cilagicin. Conditions were the same as in FIG. 2A. FIG. 2C depicts representative results demonstrating cell lysis in cilagicin treated S. aureus cultures that were monitored using SYTOX dyes. Data are presented as the mean of three independent experiments ±SD. FIG. 2D depicts representative results demonstrating membrane depolarization in cilagicin treated S. aureus cultures was monitored using DiSC3(5) dyes. Data are presented as the mean of three independent experiments ±SD. FIG. 2E depicts representation results demonstrating accumulation of UDP-MurNAc-pentapeptide after treating S. aureus cultures with cilagicin (1×MIC) was monitored by LCMS. DMSO and vancomycin (10×MIC) treated cultures were examined as controls. UDP-MurNAc-pentapeptide corresponds to (M−H)⁻=1148.53 and (M−2H)²⁻=573.87. FIG. 2F depicts representative results demonstrating fold change in cilagicin MIC upon treatment of S. aureus with 5-fold molar excess of different lipid II intermediates. The peptidoglycan mixture was added at 100 μg/mL. Data are representative of two independent experiments.

FIG. 3, comprising FIG. 3A through FIG. 3F, depicts representative results demonstrating the interaction of cilagicin with C55-P and C55-PP. FIG. 3A depicts representative results demonstrating fold change in MIC of cilagicin treated cultures of S. aureus USA300 in the presence of different concentrations of C55-P. The highest concentration tested was 32× the MIC. Data from two independent experiments are presented. FIG. 3B depicts representative results demonstrating fold change in MIC of cilagicin treated cultures of S. aureus USA300 in the presence of different concentrations of C55-PP. The highest concentration tested was 32× the MIC. Data from two independent experiments are presented. FIG. 3C depicts representative results demonstrating isothermal titration calorimetry data for cilagicin or its inactive analog C3b interacting with either C55-P. Two independent experiments were performed with similar results. FIG. 3D depicts representative results demonstrating isothermal titration calorimetry data for cilagicin or its inactive analog C3b interacting with either C55-PP. Two independent experiments were performed with similar results. FIG. 3E depicts a schematic representation of the role of C55-P and C55-PP in Gram-positive cell wall biosynthesis. FIG. 3F depicts representative results demonstrating resistance acquisition during serial passaging of S. aureus USA300 in the presence of sub-MIC levels of cilagicin, bacitracin or amphomycin. Data shown represent the mean of three independent experiments ±SEM. Inset: The MIC fold increase of cilagicin against bacitracin (green) and amphomycin (red) resistant strains on 25 days.

FIG. 4, comprising FIG. 4A through FIG. 4D, depicts representative results demonstrating the cilagicin BP activity in a murine neutropenic thigh infection model. FIG. 4A depicts representative anti S. aureus activity of cilagicin analogs with different lipid substituents in the presence of 10% serum. Blue: MIC<4 μg/mL or no change in MIC in the presence of serum. FIG. 4B depicts a schematic representation of the structure of cilagicin BP (L1). FIG. 4C depicts representative results demonstrating neutropenic thigh infection model using S. aureus USA300. FIG. 4D depicts representative results demonstrating neutropenic thigh infection model using S. pyrogens ATCC19615. Two hours post infection with a fresh bacterial suspension (1×10⁶ CFU), vehicle (10% DMSO, TID), vancomycin (40 mg/kg, TID) or cilagicin BP (40 mg/kg, TID) were delivered by IP injection. After 24 hours post infection, CFUs were determined from homogenized thigh tissue samples. Significant differences between groups were analyzed by one-way analysis of variance (ANOVA) (***P=0.0001, ****P<0.0001) (n=4 mice, n=8 thighs). Mean CFU counts and SD are shown.

FIG. 5 depicts a schematic representation of cilagicin.

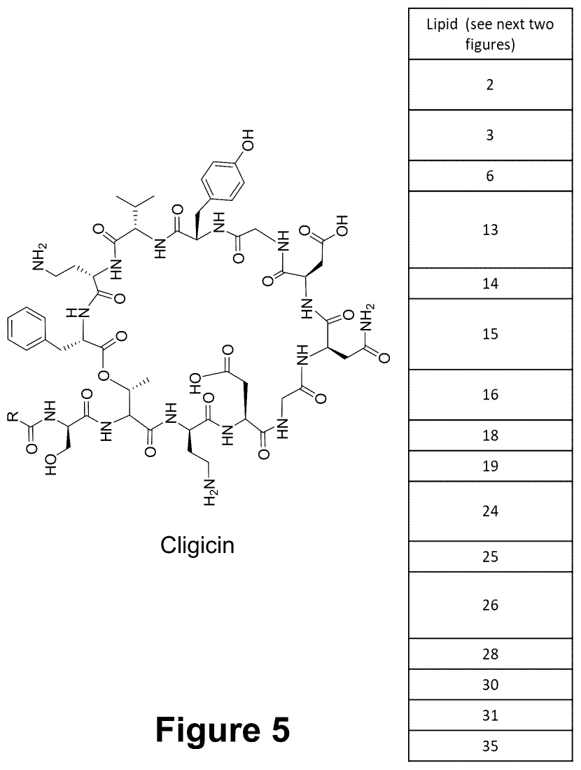

FIG. 6 depicts a schematic representation of additional lipids references in FIG. 5.

FIG. 7 depicts a schematic representation of additional unexpected lipids references in FIG. 5.

FIG. 8 depicts a schematic representation of cilagicin analogs.

FIG. 9 depicts representative results demonstrating the pharmacological assessment of cilagicin via intraperitoneal (IP), intravenous (IV) or subcutaneous (SC) injection. Data shown represent the mean of three independent mice ±SD.

FIG. 10, comprising FIG. 10A through FIG. 10C, depicts representative results demonstrating the activity of cilagicin BP. FIG. 10A depicts representative MIC of cilagicin BP against S. aureus USA300 in the presence of C55-P or C55-PP. Each compound was added in a 5:1 molar ratio compared to cilagicin BP. Data are from two independent experiments is presented. FIG. 10B depicts representative MTT assay of cilagicin BP against HEK293 cell. The experiments were performed in triplicates (n=3) and repeated two independent times (n=2). Mean and SD was shown. FIG. 10C depicts representative results of hemolytic assay of cilagicin and its analogs (cilagicin BP and L5). The figure is representative of two independent assays.

FIG. 11, comprising FIG. 11A and FIG. 11B, depicts representative results of LCMS analysis of cilagicin. FIG. 11A depicts representative HPLC chromatogram of cilagicin. FIG. 11B depicts representative high-resolution mass spectra (HRMS) of cilagicin.

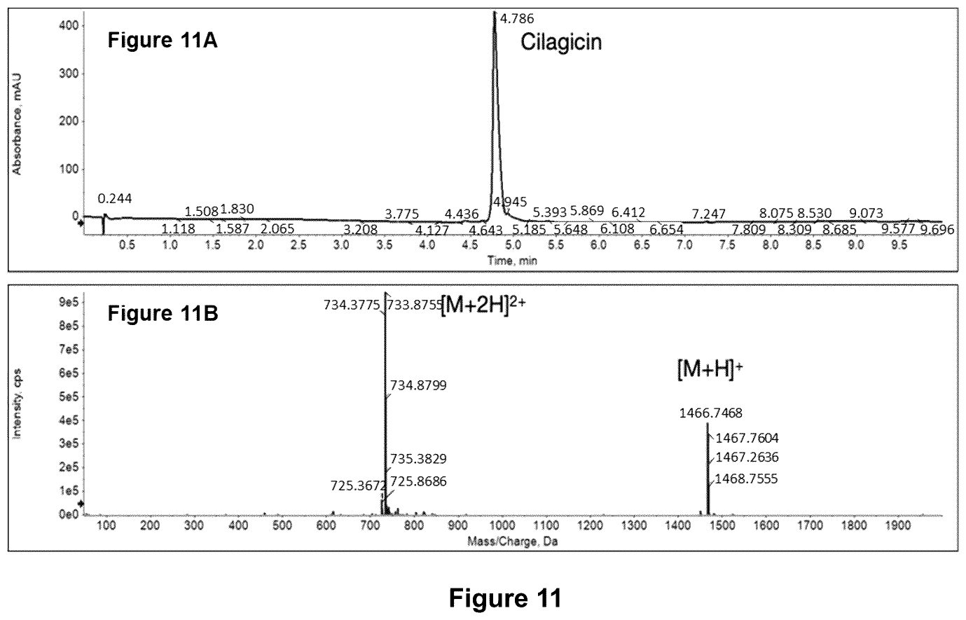

FIG. 12 depicts representative ¹H NMR (DMSO-d6, 600 MHz) spectrum of cilagicin.

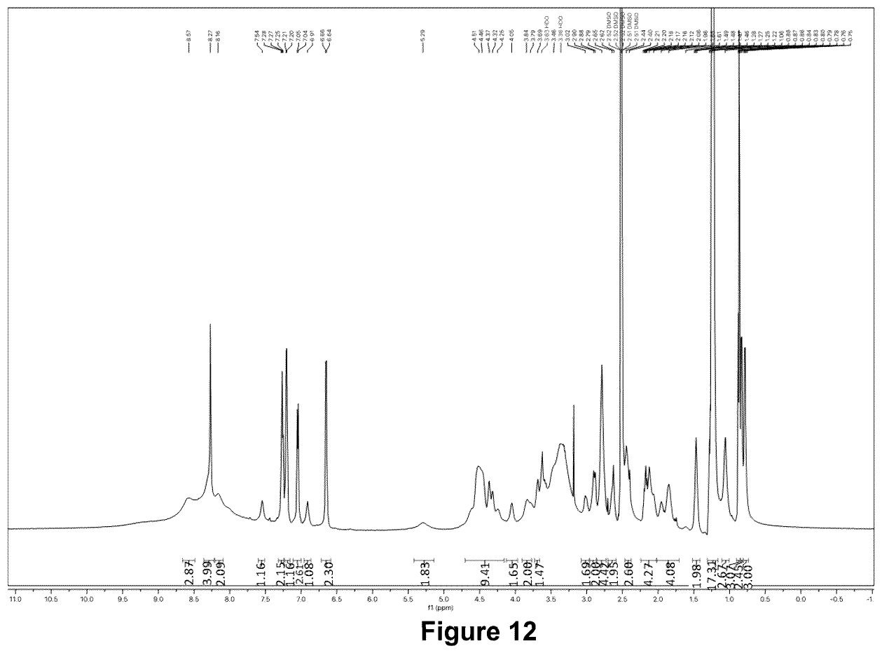

FIG. 13 depicts representative ¹³C NMR (DMSO-d6, 600 MHz) spectrum of cilagicin.

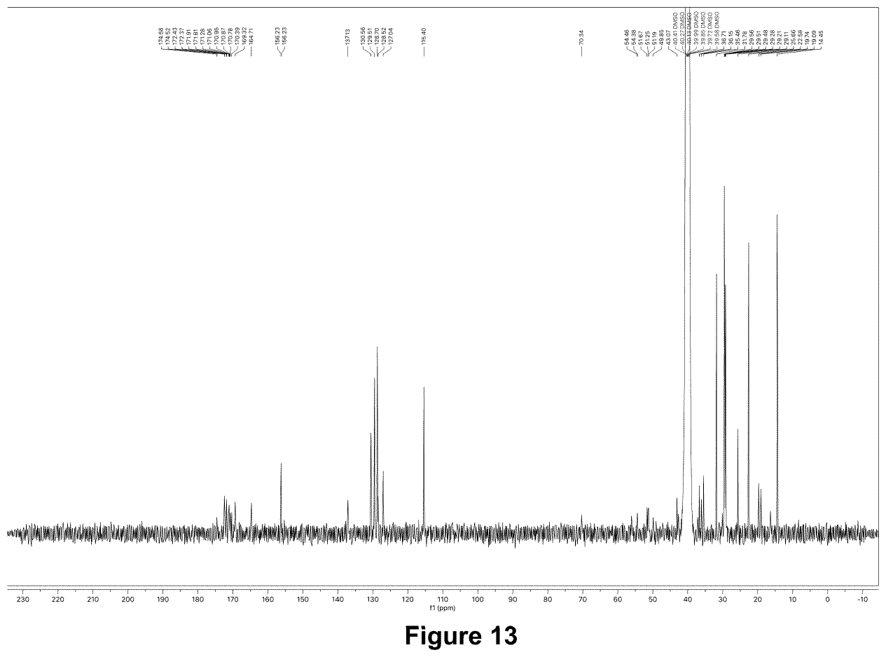

FIG. 14, comprising FIG. 14A and FIG. 14B, depicts representative results of LCMS analysis of cilagicin BP. FIG. 14A depicts representative HPLC chromatogram of cilagicin BP. FIG. 14B depicts representative high-resolution mass spectra (HRMS) of Cilagicin BP.

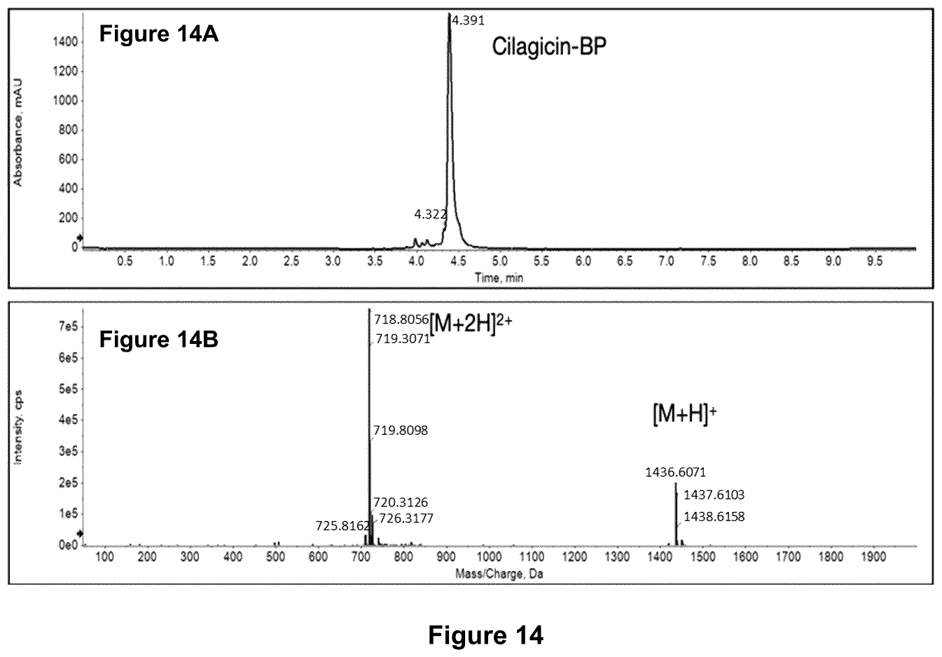

FIG. 15 depicts representative ¹H NMR (DMSO-d6, 600 MHz) spectrum of cilagicin BP.

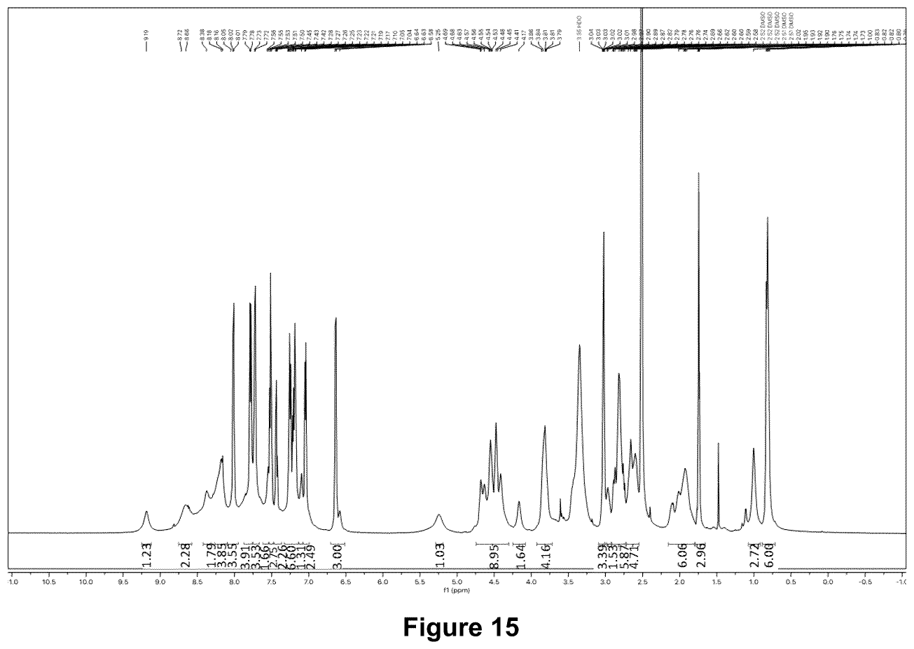

FIG. 16 depicts representative ¹³C NMR (DMSO-d6, 600 MHz) spectrum of cilagicin BP.

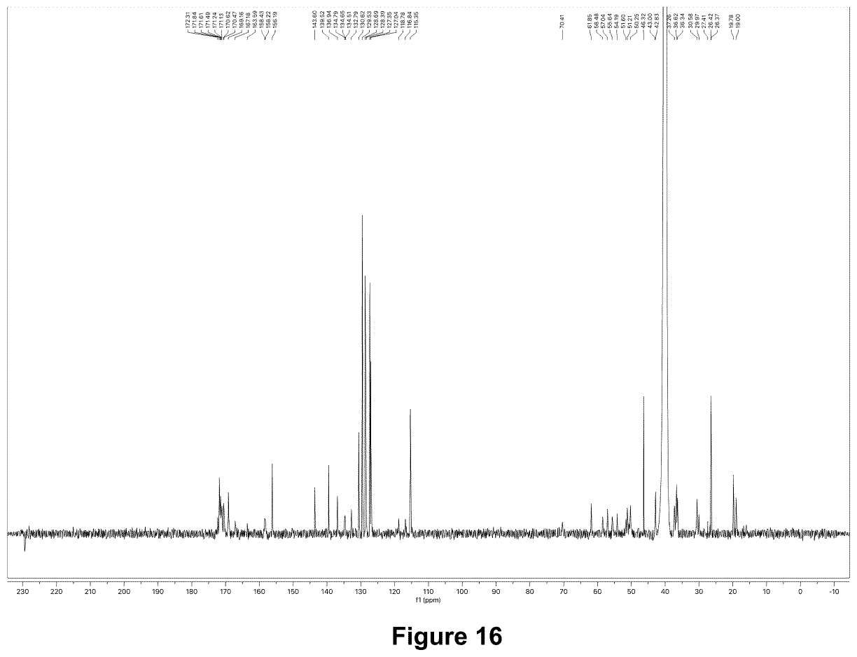

### DETAILED DESCRIPTION

The present invention is based, in part, on the unexpected discovery of cilagicin compounds as antibiotics which have activity against multidrug resistant pathogens. In one aspect, the present invention provides compounds or a therapeutic compound comprising a desired activity. In one embodiment, the compound is an antibiotic. In one embodiment, the antibiotic compound of the invention can be used in the treatment of bacterial infections. In one embodiment, the antibiotic compound of the invention can be used in the treatment of gram positive bacterial infections. In certain embodiments, the use of the antibiotic compound of the invention in the treatment of bacterial infections optionally includes a pharmaceutically acceptable carrier, excipient, or adjuvant.

In one embodiment, the compound can be biosynthesized via heterologous expression of a biosynthetic gene. Thus, in one aspect, the invention provides compounds and methods for synthesizing cilagicin compounds. In one embodiment, the invention provides a nucleic acid encoding cilagicin compounds. In one embodiment, the nucleic acid is an isolated nucleic acid. In one embodiment, the nucleic acid is transformed into a cell.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of 20%, ±10%, ±5%, ±1%, or ±0.1% from the specified value, as such variations are appropriate to perform the disclosed methods.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

To “treat” a disease as the term is used herein, means to reduce the frequency or severity of at least one sign or symptom of a disease or disorder experienced by a subject.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

“Parenteral” administration of a composition includes, e.g., subcutaneous (s.c.), intravenous (i.v.), intramuscular (i.m.), or intrastemal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

The term, “biologically active” or “bioactive” can mean, but is in no way limited to, the ability of an agent or compound to effectuate a physiological change or response. The response may be detected, for example, at the cellular level, for example, as a change in growth and/or viability, gene expression, protein quantity, protein modification, protein activity, or combination thereof; at the tissue level; at the systemic level; or at the organism level. For example, as used herein, biologically active molecules include but are not limited to any substance intended for diagnosis, cure, mitigation, treatment, or prevention of disease in humans or other animals, or to otherwise enhance physical or mental well-being of humans or animals. Examples of biologically active molecules include, but are not limited to, peptides, proteins, enzymes, small molecule drugs, dyes, lipids, nucleosides, oligonucleotides, cells, viruses, liposomes, microparticles and micelles. Classes of biologically active agents that are suitable for use with the invention include, but are not limited to, antibiotics, fungicides, anti-viral agents, anti-inflammatory agents, anti-tumor agents, cardiovascular agents, anti-anxiety agents, hormones, growth factors, steroidal agents, and the like.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

An “effective amount” or “therapeutically effective amount” of a compound is that amount of compound which is sufficient to provide a beneficial effect to the subject to which the compound is administered. An “effective amount” of a delivery vehicle is that amount sufficient to effectively bind or deliver a compound.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared ×100. For example, if 6 of 10 of the positions in two sequences are matched or homologous then the two sequences are 60% homologous. By way of example, the DNA sequences ATGG and ATCC share 50% homology. Generally, a comparison is made when two sequences are aligned to give maximum homology.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

As used herein, the terms “amino acid”, “amino acidic monomer”, or “amino acid residue” refer to any of the twenty naturally occurring amino acids including synthetic amino acids with unnatural side chains and including both D and L optical isomers.

In the context of the invention, term “natural amino acid” means any amino acid which is found naturally in vivo in a living being. Natural amino acids therefore include amino acids coded by mRNA incorporated into proteins during translation but also other amino acids found naturally in vivo which are a product or by-product of a metabolic process, such as for example ornithine which is generated by the urea production process by arginase from L-arginine. In the invention, the amino acids used can therefore be natural or not. Namely, natural amino acids generally have the L configuration but also, according to the invention, an amino acid can have the L or D configuration.

A “non-naturally encoded amino acid” refers to an amino acid that is not one of the 20 common amino acids or pyrolysine or selenocysteine. The term “non-naturally encoded amino acid” includes, but is not limited to, amino acids that occur naturally by modification of a naturally encoded amino acid (including but not limited to, the 20 common amino acids or pyrolysine and selenocysteine) but are not themselves incorporated into a growing polypeptide chain by the translation complex. Examples of naturally-occurring amino acids that are not naturally-encoded include, but are not limited to, N-acetylglucosaminyl-L-serine, N-acetylglucosaminyl-L-threonine, and O-phosphotyrosine.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof. Furthermore, peptides of the invention may include amino acid mimentics, and analogs. Recombinant forms of the peptides can be produced according to standard methods and protocols which are well known to those of skill in the art, including for example, expression of recombinant proteins in prokaryotic and/or eukaryotic cells followed by one or more isolation and purification steps, and/or chemically synthesizing peptides or portions thereof using a peptide sythesizer.

The term “pharmacological composition,” “therapeutic composition,” “therapeutic formulation” or “pharmaceutically acceptable formulation” can mean, but is in no way limited to, a composition or formulation that allows for the effective distribution of an agent provided by the invention, which is in a form suitable for administration to the physical location most suitable for their desired activity, e.g., systemic administration.

Non-limiting examples of agents suitable for formulation with the, e.g., compounds provided by the instant invention include: cinnamoyl, PEG, phospholipids or lipophilic moieties, phosphorothioates, P-glycoprotein inhibitors (such as Pluronic P85) which can enhance entry of drugs into various tissues, for example the CNS (Jolliet-Riant and Tillement, 1999, Fundam. Clin. Pharmacol., 13, 16-26); biodegradable polymers, such as poly (DL-lactide-coglycolide) microspheres for sustained release delivery after implantation (Emerich, D F et al, 1999, Cell Transplant, 8, 47-58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999).

The term “pharmaceutically acceptable” or “pharmacologically acceptable” can mean, but is in no way limited to, entities and compositions that do not produce an adverse, allergic or other untoward reaction when administered to an animal, or a human, as appropriate.

The term “pharmaceutically acceptable carrier” or “pharmacologically acceptable carrier” can mean, but is in no way limited to, any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A “therapeutic” treatment is a treatment administered to a subject who exhibits signs of pathology, for the purpose of diminishing or eliminating those signs.

As used herein, “treating a disease or disorder” means reducing the frequency with which a symptom of the disease or disorder is experienced by a patient. Disease and disorder are used interchangeably herein.

The phrase “therapeutically effective amount,” as used herein, refers to an amount that is sufficient or effective to prevent or treat (delay or prevent the onset of, prevent the progression of, inhibit, decrease or reverse) a disease or condition, including alleviating symptoms of such diseases.

The term “compound,” as used herein, unless otherwise indicated, refers to any specific chemical compound disclosed herein. In one embodiment, the term also refers to stereoisomers and/or optical isomers (including racemic mixtures) or enantiomerically enriched mixtures of disclosed compounds.

As used herein, “derivatives” are compositions formed from the native compounds either directly, by modification, or by partial substitution. As used herein, “analogs” are compositions that have a structure similar to, but not identical to, the native compound.

As used herein, the term “alkyl,” by itself or as part of another substituent means, unless otherwise stated, a straight or branched chain hydrocarbon having the number of carbon atoms designated (i.e. C1-6 means one to six carbon atoms) and includes straight, branched chain, or cyclic substituent groups. Examples include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. The term “alkyl,” unless otherwise noted, is also meant to include those derivatives of alkyl defined in more detail below, such as “heteroalkyl”, “haloalkyl” and “homoalkyl”.

As used herein, the term “substituted alkyl” means alkyl, as defined above, substituted by one, two or three substituents selected from the group consisting of halogen, —OH, alkoxy, —NH₂, —N(CH₃)₂, —C(═O)OH, trifluoromethyl, —C≡N, —C(═O)O(C₁-C₄)alkyl, —C(═O)NH₂, —SO₂NH₂, —C(═NH)NH₂, and —NO₂, preferably containing one or two substituents selected from halogen, —OH, alkoxy, —NH₂, trifluoromethyl, —N(CH₃)₂, and —C(═O)OH, more preferably selected from halogen, alkoxy and —OH. Examples of substituted alkyls include, but are not limited to, 2,2-difluoropropyl, 2-carboxycyclopentyl and 3-chloropropyl.

As used herein, the term “alkylene” by itself or as part of another molecule means a divalent radical derived from an alkane, as exemplified by (—CH₂—)ₙ. By way of example only, such groups include, but are not limited to, groups having 24 or fewer carbon atoms such as the structures —CH₂CH₂— and —CH₂CH₂CH₂CH₂—. The term “alkylene,” unless otherwise noted, is also meant to include those groups described below as “heteroalkylene.”

As used herein, the terms “alkoxy,” “alkylamino” and “alkylthio” are used in their conventional sense, and refer to alkyl groups linked to molecules via an oxygen atom, an amino group, a sulfur atom, respectively.

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. Preferred are (C₁-C₃) alkoxy, particularly ethoxy and methoxy.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “cycloalkyl” refers to a mono cyclic or polycyclic non-aromatic radical, wherein each of the atoms forming the ring (i.e. skeletal atoms) is a carbon atom. In one embodiment, the cycloalkyl group is saturated or partially unsaturated. In another embodiment, the cycloalkyl group is fused with an aromatic ring. Cycloalkyl groups include groups having from 3 to 10 ring atoms. Illustrative examples of cycloalkyl groups include, but are not limited to, the following moieties:

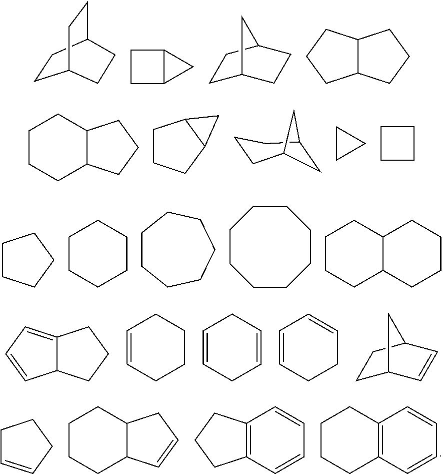

Monocyclic cycloalkyls include, but are not limited to, cyclopropyl, cyclobutyl, cyclopentyl, cyclohexyl, cycloheptyl, and cyclooctyl. Dicyclic cycloalkyls include, but are not limited to, tetrahydronaphthyl, indanyl, and tetrahydropentalene. Polycyclic cycloalkyls include adamantine and norbomane. The term cycloalkyl includes “unsaturated nonaromatic carbocyclyl” or “nonaromatic unsaturated carbocyclyl” groups, both of which refer to a nonaromatic carbocycle as defined herein, which contains at least one carbon carbon double bond or one carbon carbon triple bond.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, Si, P, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include. —O—CH₂—CH₂—CH₃, —CH₂—CH₂—CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, —CH₂—NH—OCH₃, or —CH₂—CH₂—S—S—CH₃.

As used herein, the term “heterocycle” or “heterocyclyl” or “heterocyclic” by itself or as part of another substituent means, unless otherwise stated, an unsubstituted or substituted, stable, mono- or multi-cyclic heterocyclic ring system that consists of carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. An example of a 3-membered heterocycloalkyl group includes, and is not limited to, aziridine. Examples of 4-membered heterocycloalkyl groups include, and are not limited to, azetidine and a beta lactam. Examples of 5-membered heterocycloalkyl groups include, and are not limited to, pyrrolidine, oxazolidine and thiazolidinedione. Examples of 6-membered heterocycloalkyl groups include, and are not limited to, piperidine, morpholine and piperazine. Other non-limiting examples of heterocycloalkyl groups are.

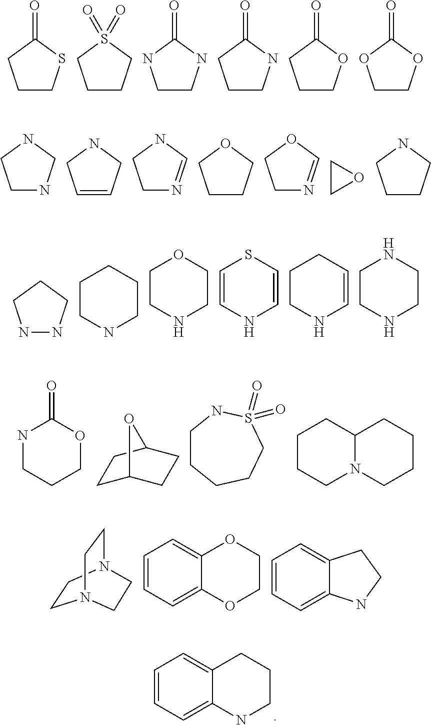

Examples of non-aromatic heterocycles include monocyclic groups such as aziridine, oxirane, thiirane, azetidine, oxetane, thietane, pyrrolidine, pyrroline, imidazoline, pyrazolidine, dioxolane, sulfolane, 2,3-dihy drofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihy dropyran, tetrahydropyran, 1,4-dioxane, 1,3-dioxane, homopiperazine, homopiperidine, 1,3-dioxepane, 4,7-dihydro-1,3-dioxepin and hexamethyleneoxide.

As used herein, the term “aromatic” refers to a carbocycle or heterocycle with one or more polyunsaturated rings and having aromatic character, i.e. having (4n+2) delocalized π (pi) electrons, where n is an integer.

As used herein, the term “aryl,” employed alone or in combination with other terms, means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl, and naphthyl. Preferred are phenyl and naphthyl, most preferred is phenyl.

As used herein, the term “aryl-(C₁-C₄)alkyl” means a functional group wherein a one to three carbon alkylene chain is attached to an aryl group, e.g., —CH₂CH₂-phenyl. Preferred is aryl-CH₂— and aryl-CH(CH₃)—. The term “substituted aryl-(C₁-C₄)alkyl” means an aryl-(C₁-C₄)alkyl functional group in which the aryl group is substituted. Preferred is substituted aryl(CH₂)—. Similarly, the term “heteroaryl-(C₁-C₄)alkyl” means a functional group wherein a one to three carbon alkylene chain is attached to a heteroaryl group, e.g., —CH₂CH₂-pyridyl. Preferred is heteroaryl-(CH₂)—. The term “substituted heteroaryl-(C₁-C₄)alkyl” means a heteroaryl-(C₁-C₄)alkyl functional group in which the heteroaryl group is substituted. Preferred is substituted heteroaryl-(CH₂)—.

Examples of heteroaryl groups include pyridyl, pyrazinyl, pyrimidinyl (particularly 2- and 4-pyrimidinyl), pyridazinyl, thienyl, furyl, pyrrolyl (particularly 2-pyrrolyl), imidazolyl, thiazolyl, oxazolyl, pyrazolyl (particularly 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles include indolyl (particularly 3-, 4-, 5-, 6- and 7-indolyl), indolinyl, quinolyl, tetrahydroquinolyl, isoquinolyl (particularly 1- and 5-isoquinolyl), 1,2,3,4-tetrahydroisoquinolyl, cinnolinyl, quinoxalinyl (particularly 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (particularly 3-, 4-, 5-, 6- and 7-benzofuryl), 2,3-dihydrobenzofuryl, 1,2-benzisoxazolyl, benzothienyl (particularly 3-, 4-, 5-, 6-, and 7-benzothienyl), benzoxazolyl, benzothiazolyl (particularly 2-benzothiazolyl and 5-benzothiazolyl), purinyl, benzimidazolyl (particularly 2-benzimidazolyl), benztriazolyl, thioxanthinyl, carbazolyl, carbolinyl, acridinyl, pyrrolizidinyl, and quinolizidinyl.

The aforementioned listing of heterocyclyl and heteroaryl moieties is intended to be representative and not limiting.

As used herein, the term “amino aryl” refers to an aryl moiety which contains an amino moiety. Such amino moieties may include, but are not limited to primary amines, secondary amines, tertiary amines, masked amines, or protected amines. Such tertiary amines, masked amines, or protected amines may be converted to primary amine or secondary amine moieties. Additionally, the amine moiety may include an amine-like moiety which has similar chemical characteristics as amine moieties, including but not limited to chemical reactivity.

As used herein, the term “substituted” means that an atom or group of atoms has replaced hydrogen as the substituent attached to another group. For aryl, aryl-(C₁-C₄)alkyl and heterocyclyl groups, the term “substituted” as applied to the rings of these groups refers to any level of substitution, namely mono-, di-, tri-, tetra-, or penta-substitution, where such substitution is permitted. The substituents are independently selected, and substitution may be at any chemically accessible position. In one embodiment, the substituents vary in number between one and four. In another embodiment, the substituents vary in number between one and three. In yet another embodiment, the substituents vary in number between one and two. In yet another embodiment, the substituents are independently selected from the group consisting of C1-6 alkyl, —OH, C1-6 alkoxy, halo, amino, acetamido and nitro. In yet another embodiment, the substituents are independently selected from the group consisting of C1-6 alkyl, C1-6 alkoxy, halo, acetamido, and nitro. As used herein, where a substituent is an alkyl or alkoxy group, the carbon chain may be branched, straight or cyclic, with straight being preferred.

As used herein, the term “optionally substituted” means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

In one embodiment, the substituents are independently selected from the group consisting of oxo, halogen, —CN, —NH₂, —OH, —NH(CH₃), —N(CH₃)₂, alkyl (including straight chain, branched and/or unsaturated alkyl), substituted or unsubstituted cycloalkyl, substituted or unsubstituted heterocycloalkyl, fluoro alkyl, substituted or unsubstituted heteroalkyl, substituted or unsubstituted alkoxy, fluoroalkoxy, —S-alkyl, S(═O)₂alkyl, —C(═O)NH(substituted or unsubstituted alkyl, or substituted or unsubstituted phenyl), —C(═O)N(H or alkyl)₂, —OC(═O)N(substituted or unsubstituted alkyl)₂, —NHC(═O)NH(substituted or unsubstituted alkyl, or substituted or unsubstituted phenyl), —NHC(═O)alkyl, —N(substituted or unsubstituted alkyl)C(═O)(substituted or unsubstituted alkyl), —NHC(═O)(substituted or unsubstituted alkyl), —C(OH)(substituted or unsubstituted alkyl)₂, and —C(NH₂)(substituted or unsubstituted alkyl)₂. In another embodiment, by way of example, an optional substituent is selected from oxo, fluorine, chlorine, bromine, iodine, —CN, —NH₂, —OH, —NH(CH₃), —N(CH₃)₂, —CH₃, —CH₂CH₃, —CH(CH₃)₂, —CF₃, —CH₂CF₃, —OCH₃, —OCH₂CH₃, —OCH(CH₃)₂, —OCF₃, —OCH₂CF₃, —S(═O)₂—CH₃, —C(═O)NH₂, —C(═O)—NHCH₃, —NHC(═O)NHCH₃, —C(═O)CH₃, —ON(O)₂, and —C(═O)OH. In yet one embodiment, the substituents are independently selected from the group consisting of C₁₋₆ alkyl, —OH, C₁₋₆ alkoxy, halo, amino, acetamido, oxo and nitro. In yet another embodiment, the substituents are independently selected from the group consisting of C₁₋₆ alkyl, C₁₋₆ alkoxy, halo, acetamido, and nitro. As used herein, where a substituent is an alkyl or alkoxy group, the carbon chain may be branched, straight or cyclic.

As used herein, the term “analog,” “analogue,” or “derivative” is meant to refer to a chemical compound or molecule made from a parent compound or molecule by one or more chemical reactions. As such, an analog can be a structure having a structure similar to that of the small molecule therapeutic agents described herein or can be based on a scaffold of a small molecule therapeutic agents described herein, but differing from it in respect to certain components or structural makeup, which may have a similar or opposite action metabolically.

An analog or derivative can also be a small molecule that differs in structure from the reference molecule, but retains the essential properties of the reference molecule. An analog or derivative may change its interaction with certain other molecules relative to the reference molecule. An analog or derivative molecule may also include a salt, an adduct, tautomer, isomer, or other variant of the reference molecule.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention is based, in part, on the unexpected discovery of cilagicin compounds as antibiotics which have activity against multidrug resistant pathogens. In one embodiment, the present invention provides compounds or a therapeutic compound comprising a desired activity. In one embodiment, the compound is an antibiotic. In one embodiment, the antibiotic compound of the invention can be used in the treatment of bacterial infections. In one embodiment, the antibiotic compound of the invention can be used in the treatment of gram positive bacterial infections. In certain embodiments, the use of the antibiotic compound of the invention in the treatment of bacterial infections optionally includes a pharmaceutically acceptable carrier, excipient or adjuvant.

In one embodiment, the compound can be biosynthesized via heterologous expression of a biosynthetic gene. Thus, in one aspect, the invention provides compounds and methods for synthesizing cilagicin compounds. In one embodiment, the invention provides a nucleic acid encoding cilagicin compounds. In one embodiment, the nucleic acid is an isolated nucleic acid. In one embodiment, the nucleic acid is transformed into a cell.

### Compounds

In one aspect, the present invention provides a compound or a racemate, an enantiomer, a diastereomer thereof, a pharmaceutically acceptable salt, or a derivative thereof comprising the amino acid sequence (D(X^{A})ₐK. In one aspect of the invention, the compound inhibits cell wall biosynthesis. In one aspect of the invention, the compound specifically binds at least one undecaprenyl phosphorylate.

In one aspect of the invention, the compound is a linear compound.

In another aspect of the invention, the compound is a cyclic compound.

In one embodiment, the compound is a zwitterion. In some embodiments, the zwitterion comprises at least two positively charged residues and at least two negatively charged residues.

In some embodiments, each occurrence of X^{A} is independently selected from a natural amino acid, functionalized natural amino acid, unnatural amino acid, functionalized unnatural amino acid, or any combination thereof.

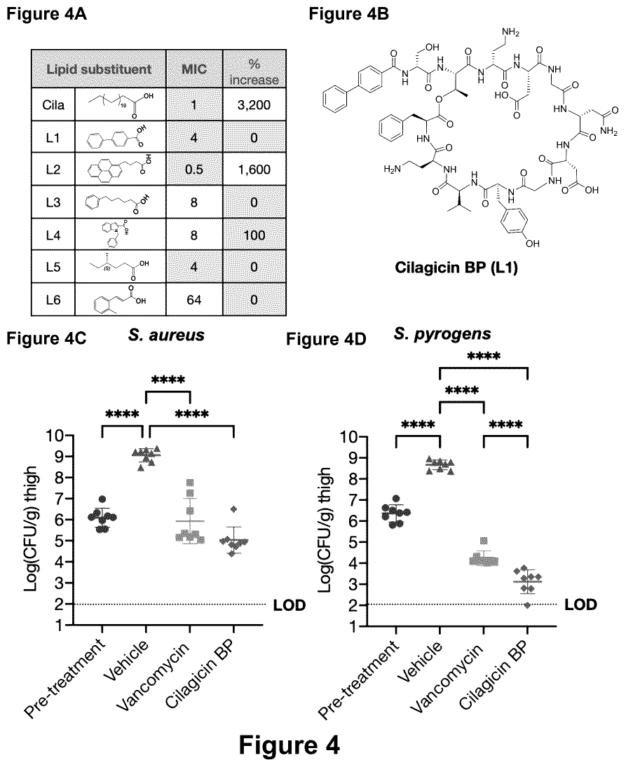

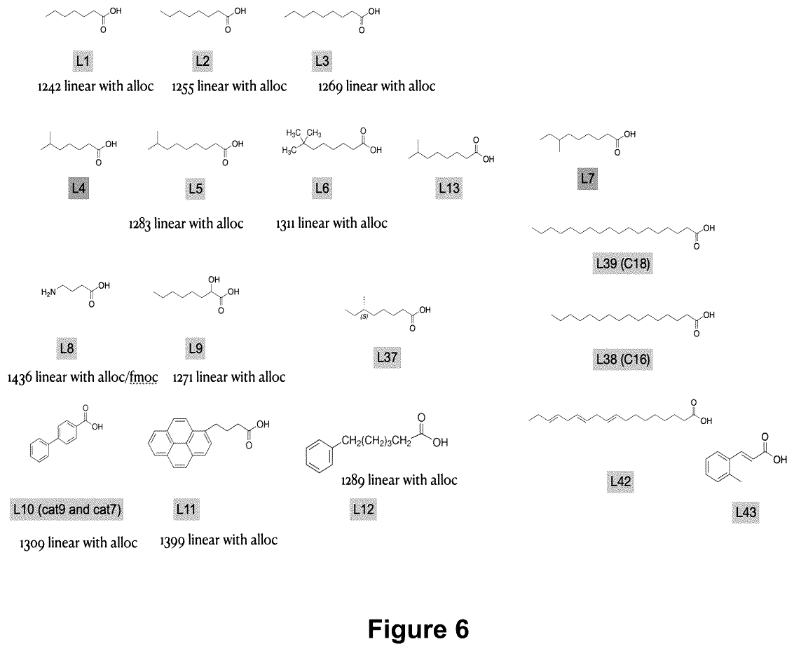

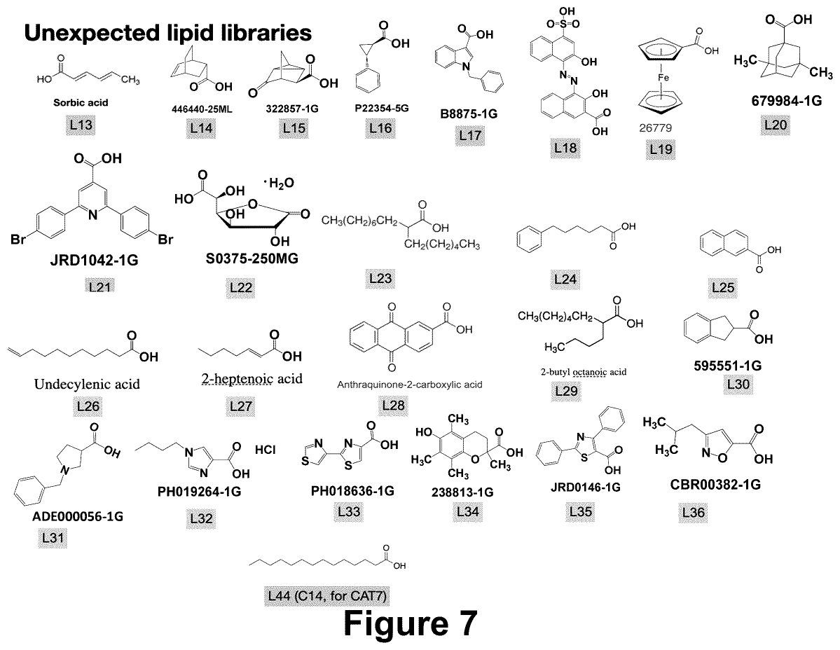

In some embodiments, the functionalized natural amino acid comprises a functional group selected from at least one selected from FIG. 4A, at least one selected from FIG. 6, at least one selected from FIG. 7, or any combination thereof. In some embodiments, the functionalized unnatural amino acid comprises a functional group selected from at least one selected from FIG. 4A, at least one selected from FIG. 6, at least one selected from FIG. 7, or any combination thereof.

In some embodiments, a is independently an integer from 8 to 100. For example, in one embodiment, a is an integer of 8. In one embodiment, a is an integer of 9. In one embodiment, a is an integer of 10. In one embodiment, a is an integer of 11. In one embodiment, a is an integer of 12. In one embodiment, a is an integer of 13. In one embodiment, a is an integer of 14. In one embodiment, a is an integer of 15. In one embodiment, a is an integer of 16. In one embodiment, a is an integer of 17. In one embodiment, a is an integer of 18. In one embodiment, a is an integer of 19. In one embodiment, a is an integer of 20. In one embodiment, a is an integer of 30. In one embodiment, a is an integer of 40. In one embodiment, a is an integer of 50. In one embodiment, a is an integer of 60. In one embodiment, a is an integer of 70. In one embodiment, a is an integer of 80. In one embodiment, a is an integer of 90. In one embodiment, a is an integer of 100.

In some embodiments, the amino acid sequence D(X^{A})ₐK comprises an amino acid sequence selected from at least one amino acid sequence as set forth in SEQ ID NOs: 1-12 or a fragment thereof, or any combination thereof.

In one embodiment, the compound is a compound of general Formula (I)

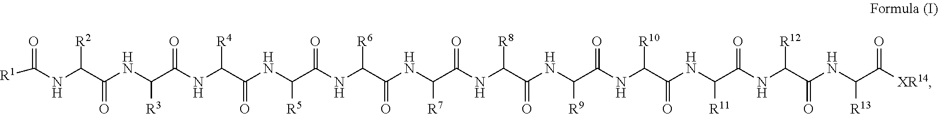

or a racemate, an enantiomer, a diastereomer, a pharmaceutically acceptable salt, or a derivative thereof.

In some embodiments, R² and X are optionally fused or joined to form a ring. Thus, in one embodiment, the compound having the structure of Formula (I) is a compound having the structure Formula (II)

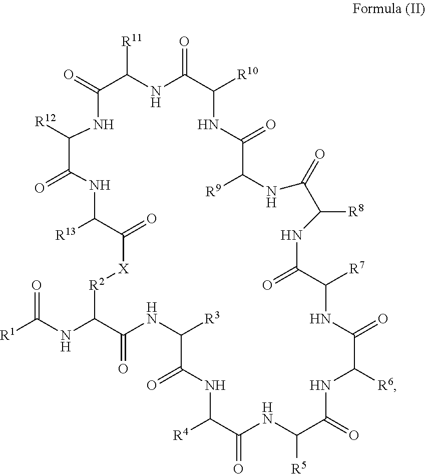

or a racemate, an enantiomer, a diastereomer, a pharmaceutically acceptable salt, or a derivative thereof.

In some embodiments, R³ and X are optionally fused or joined to form a ring. Thus, in one embodiment, the compound having the structure of Formula (I) is a compound having the structure Formula (III)

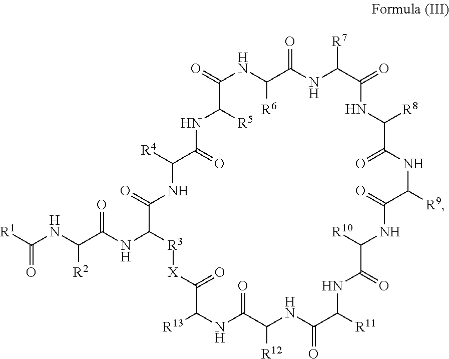

or a racemate, an enantiomer, a diastereomer, a pharmaceutically acceptable salt, or a derivative thereof.

In some embodiments, R⁴ and X are optionally fused or joined to form a ring. Thus, in one embodiment, the compound having the structure of Formula (I) is a compound having the structure Formula (IV)

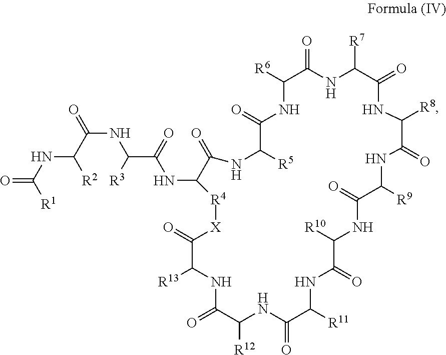

or a racemate, an enantiomer, a diastereomer, a pharmaceutically acceptable salt, or a derivative thereof.

In some embodiments, X is selected from O, S, N(R¹⁴), or any combination thereof.

In some embodiments, each occurrence of R¹, R², R³, R⁴, R⁵, R⁶, R⁷, R⁸, R⁹, R¹⁰, R¹¹, R¹², R¹³, and R¹⁴ is independently selected from hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, or any combinations thereof.

For example, in some embodiments, each occurrence of R¹, R², R³, R⁴, R⁵, R⁶, R⁷, R⁸, R⁹, R¹⁰, R¹¹, R¹², R¹³ and R¹⁴ is independently selected from hydrogen, alkyl, alkenyl, aryl, aryl alkyl, aminoalkyl, hydroxyalkyl, or any combination thereof. In other embodiments, each occurrence of R¹, R², R³, R⁴, R⁵, R⁶, R⁷, R⁸, R⁹, R¹⁰, R¹¹, R¹², R¹³, and R¹⁴ is independently selected from hydrogen, linear C₁-C₁₀ alkyl, branched C₁-C₁₀ alkyl, linear aryl-C₁-C₁₀ alkyl, branched aryl-C₁-C₁₀ alkyl, linear amino-C₁-C₁₀ alkyl, amino-branched C₁-C₁₀ alkyl, linear hydroxy-C₁-C₁₀ alkyl, hydroxy-branched C₁-C₁₀ alkyl, linear C₁-C₁₀ alkenyl, branched C₁-C₁₀ alkenyl, linear aryl-C₁-C₁₀ alkenyl, branched aryl-C₁-C₁₀ alkenyl, linear amino-C₁-C₁₀ alkenyl, amino-branched C₁-C₁₀ alkenyl, linear hydroxy-C₁-C₁₀ alkenyl, hydroxy-branched C₁-C₁₀ alkenyl, or any combination thereof.

For example, in some embodiments, the compound of the present invention is selected from

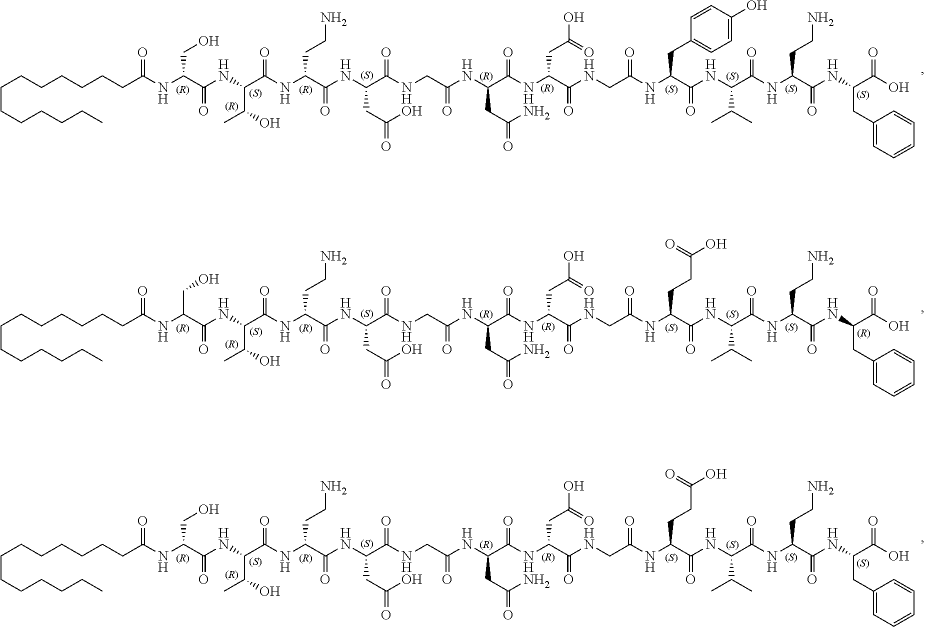

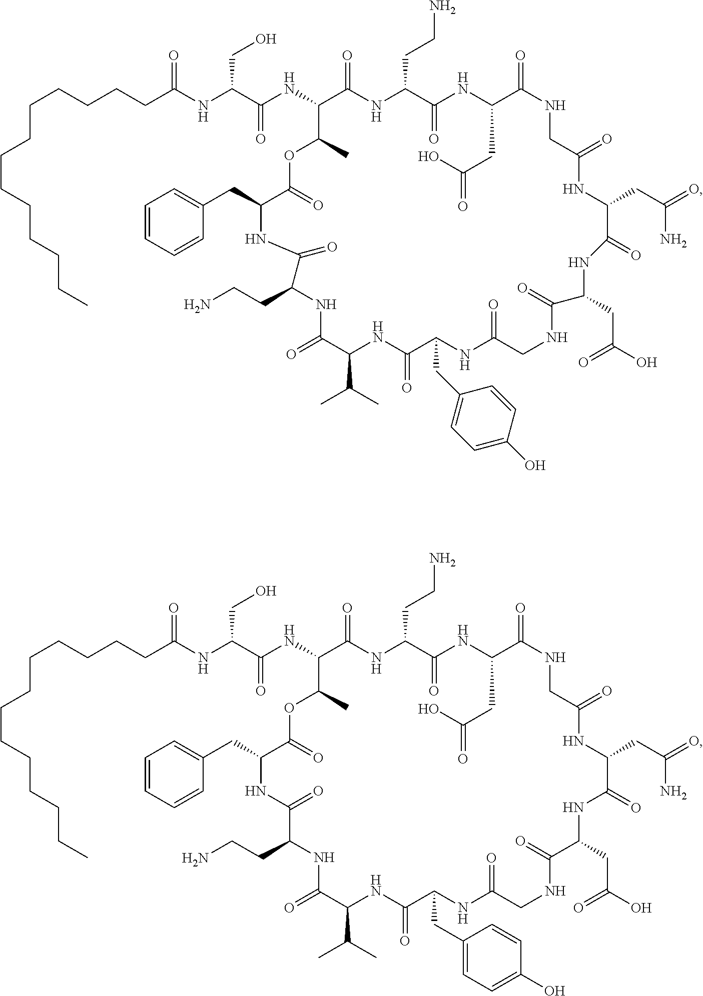 [SMILES: CCCCCCCCCCCCCC(=O)N[C@H](CO)C(=O)N[C@@H]1C(=O)N[C@H](CCN)C(=O)N[C@@H](CC(=O)O)C(=O)NCC(=O)N[C@H](CC(N)=O)C(=O)N[C@H](CC(=O)O)C(=O)NCC(=O)N[C@@H](Cc2ccc(O)cc2)C(=O)N[C@@H](C(C)C)C(=O)N[C@@H](CCN)C(=O)N[C@@H](Cc2ccccc2)C(=O)O[C@@H]1C ; CCCCCCCCCCCCCC(=O)N[C@H](CO)C(=O)N[C@@H]1C(=O)N[C@H](CCN)C(=O)N[C@@H](CC(=O)O)C(=O)NCC(=O)N[C@H](CC(N)=O)C(=O)N[C@H](CC(=O)O)C(=O)NCC(=O)N[C@@H](Cc2ccc(O)cc2)C(=O)N[C@@H](C(C)C)C(=O)N[C@@H](CCN)C(=O)N[C@H](Cc2ccccc2)C(=O)O[C@@H]1C]

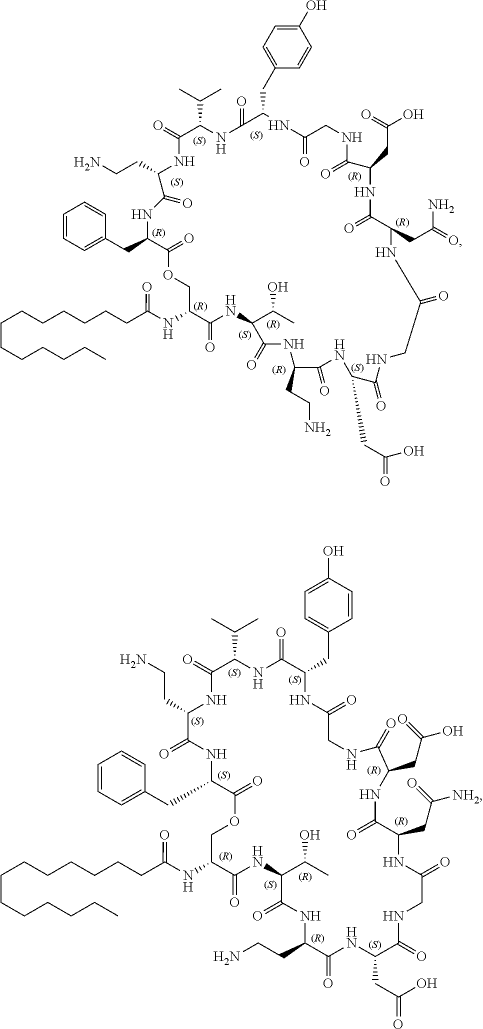

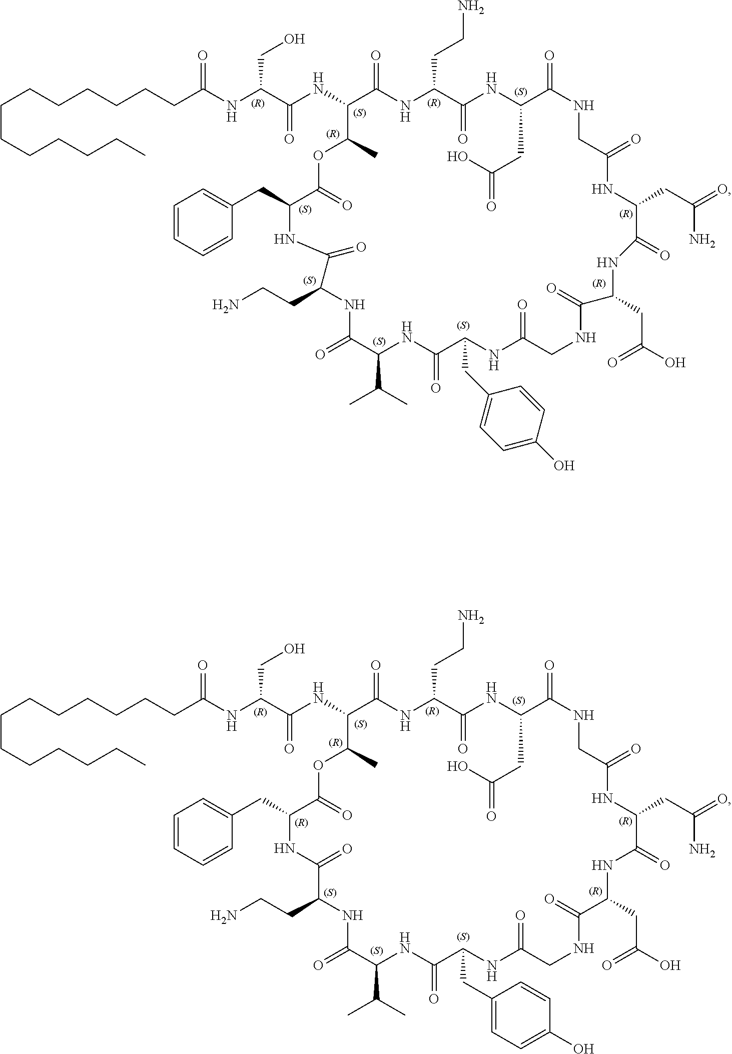

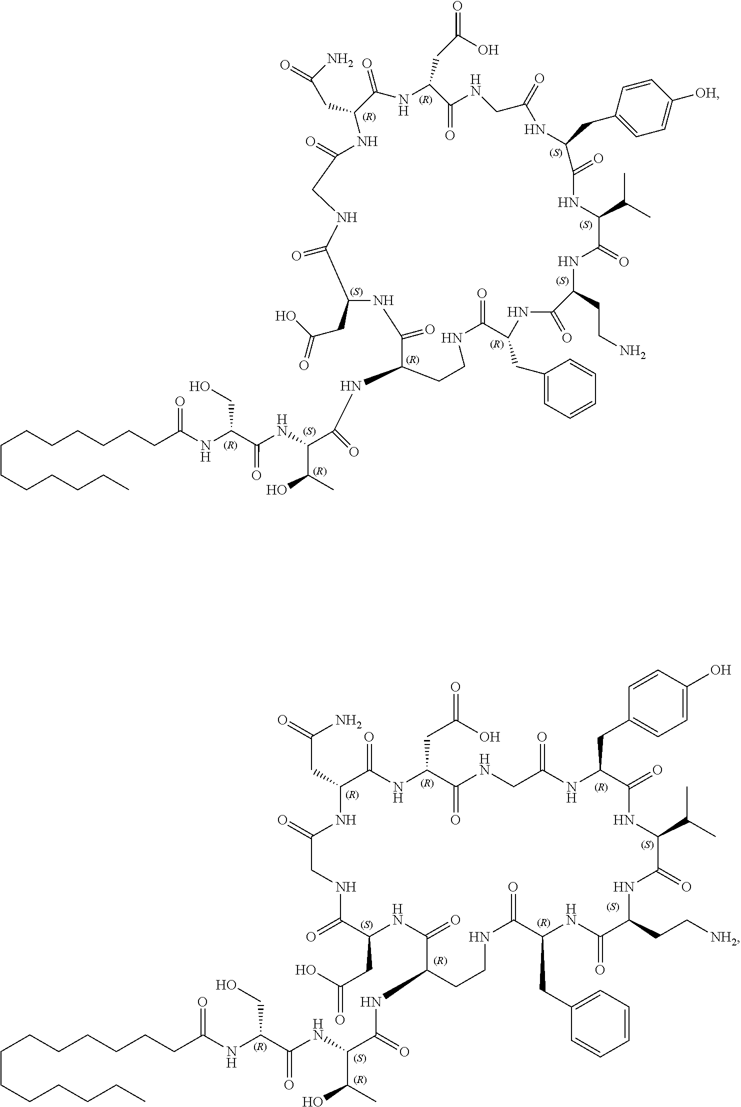

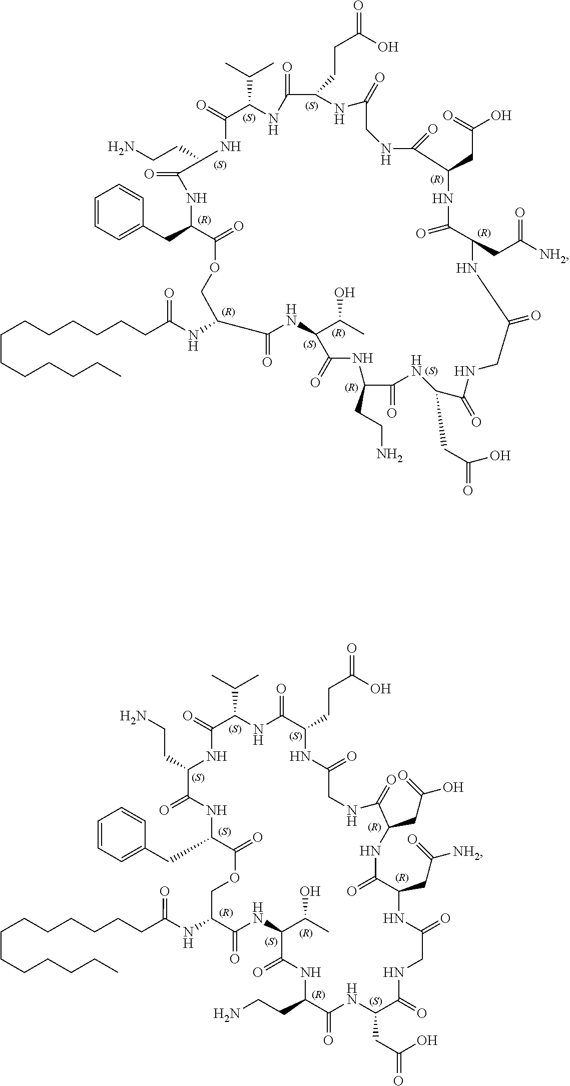

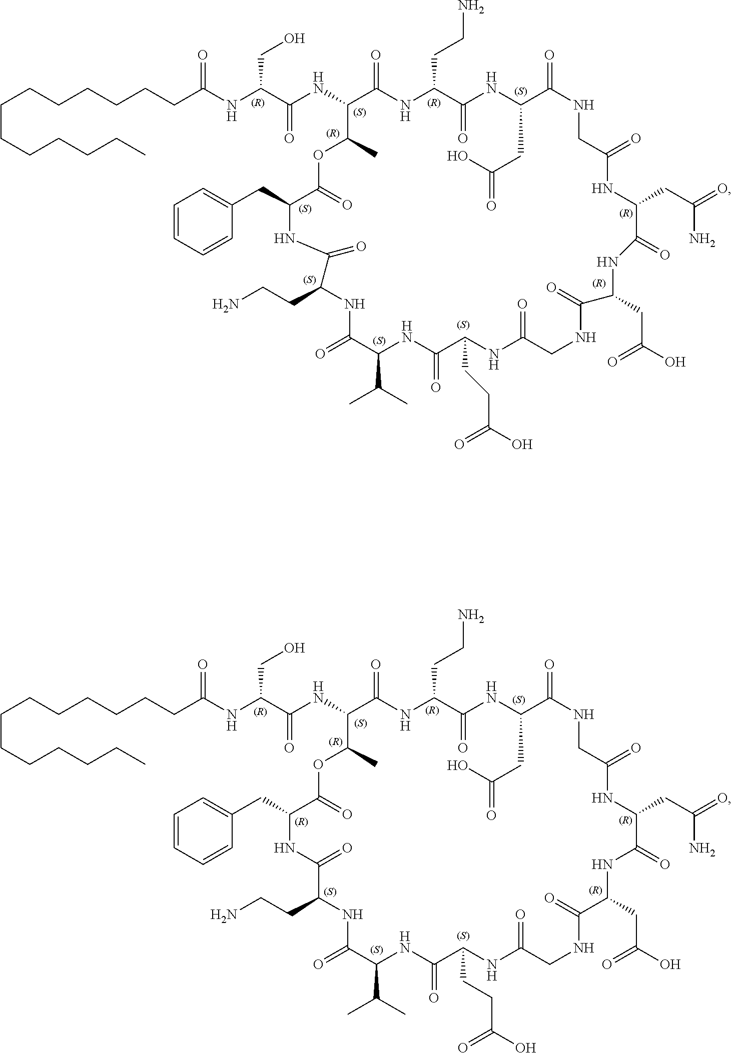

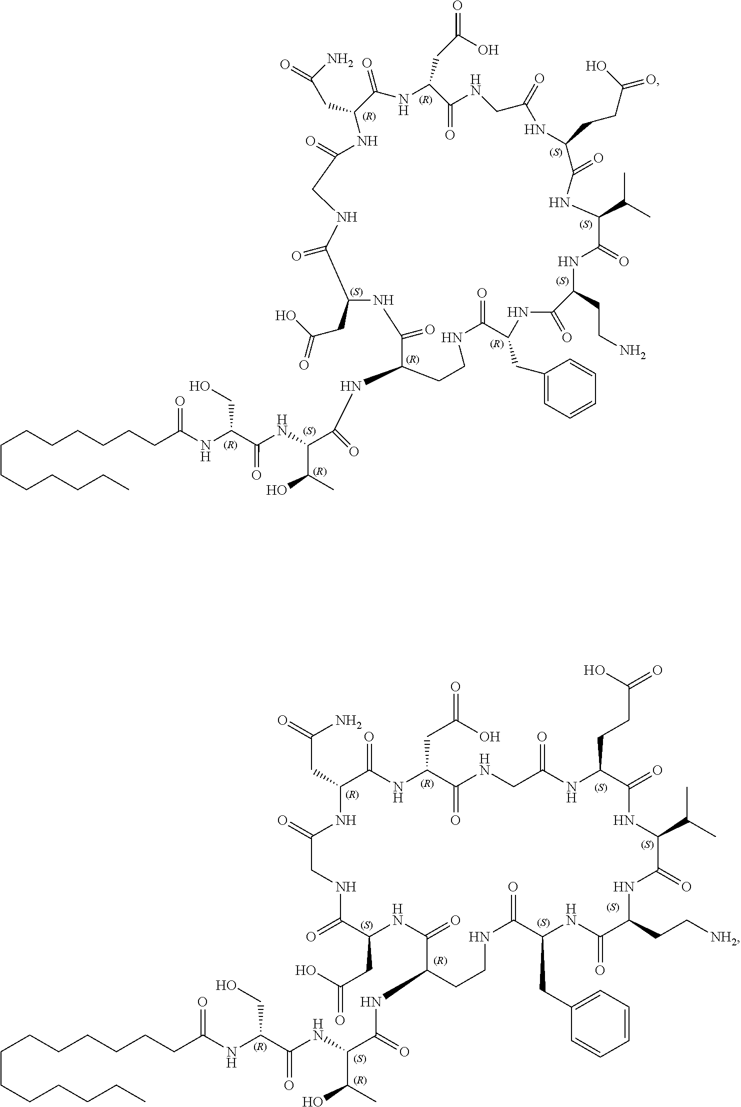

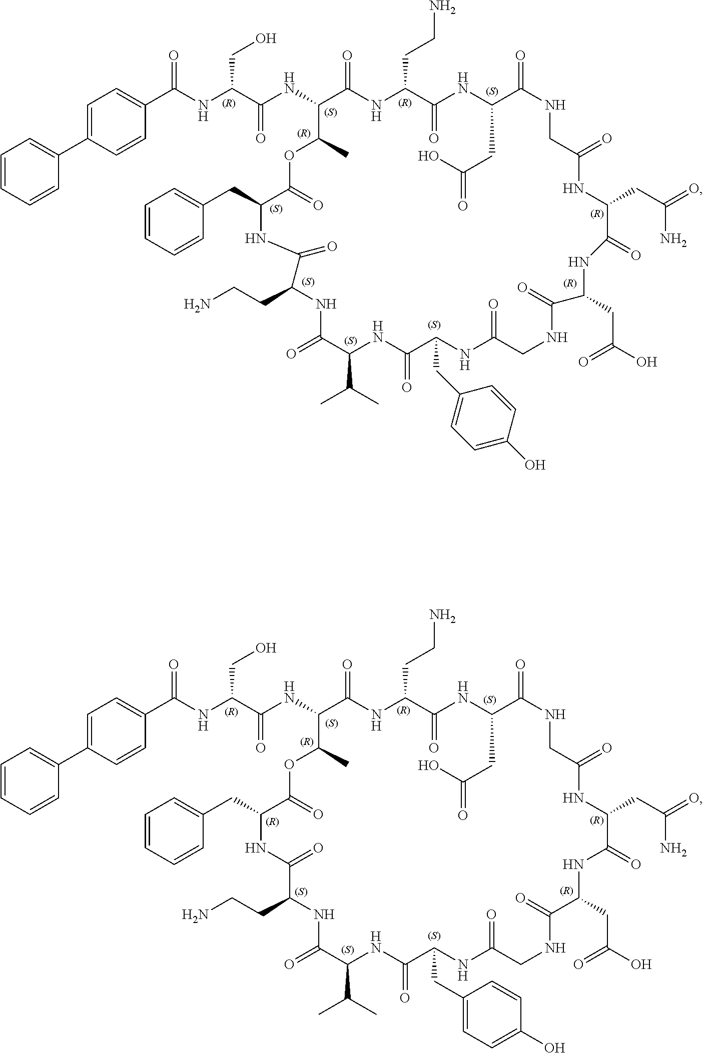

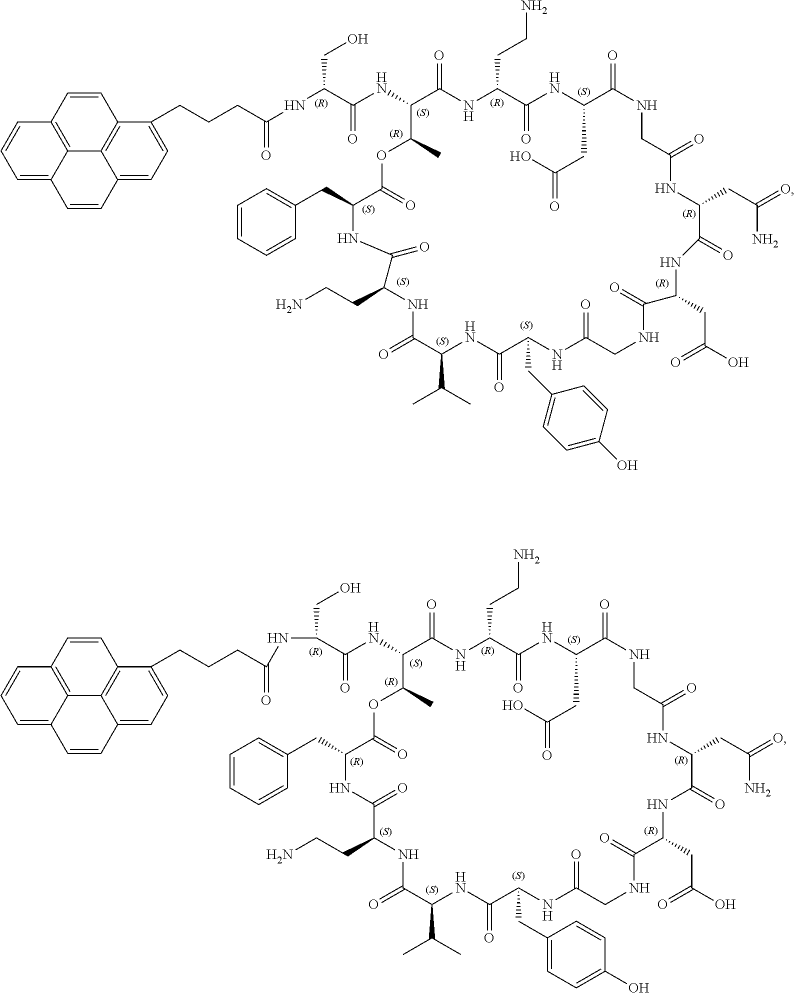

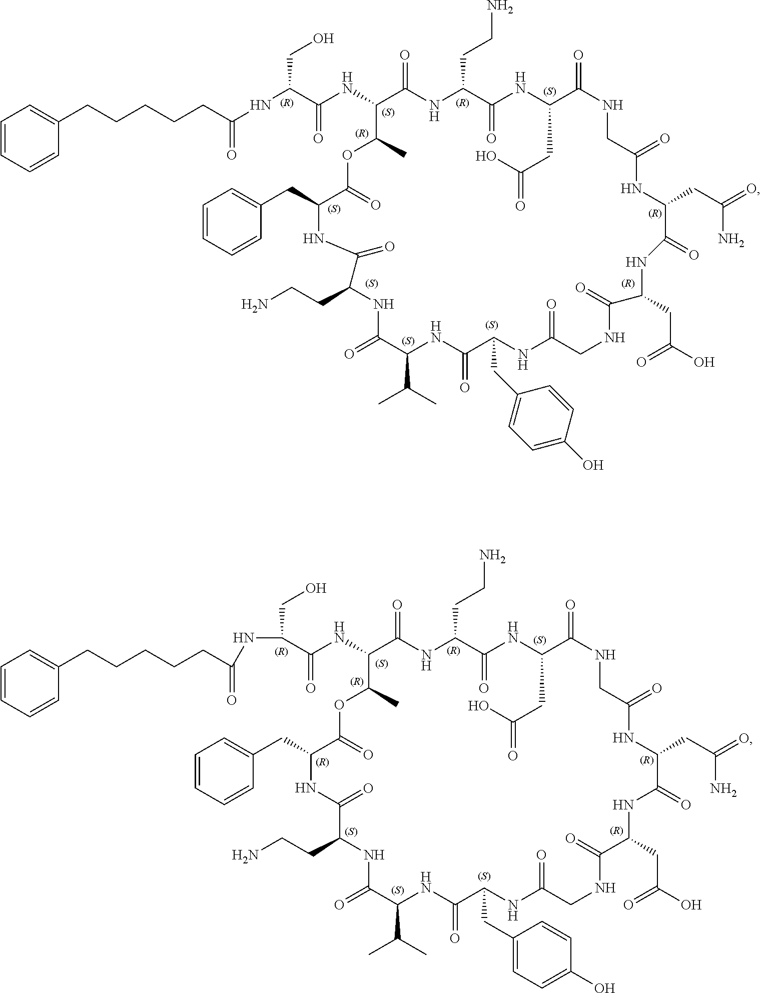

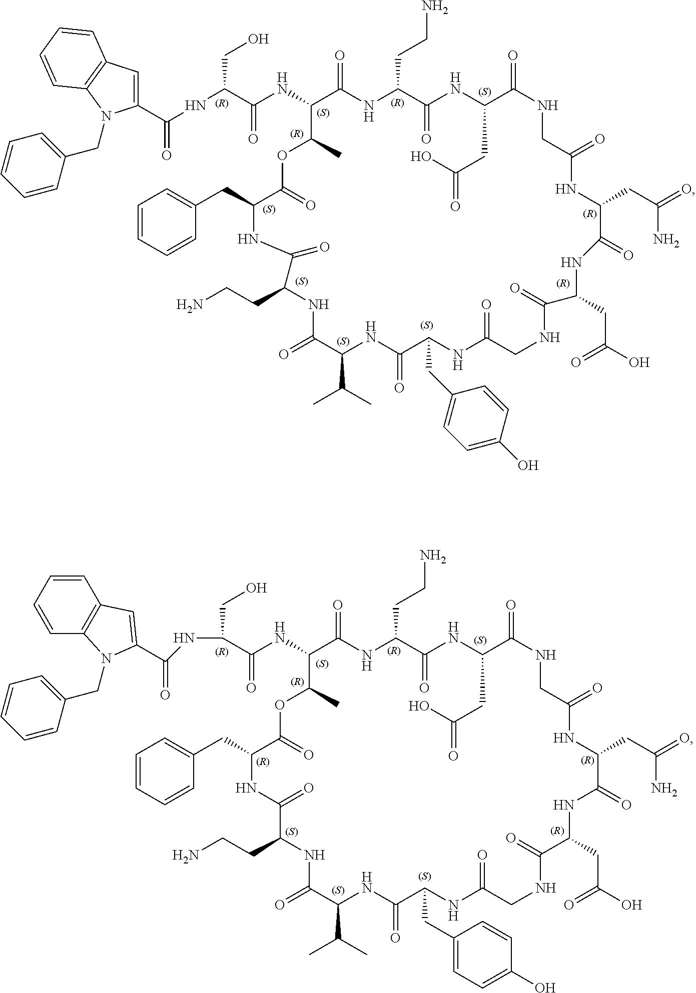

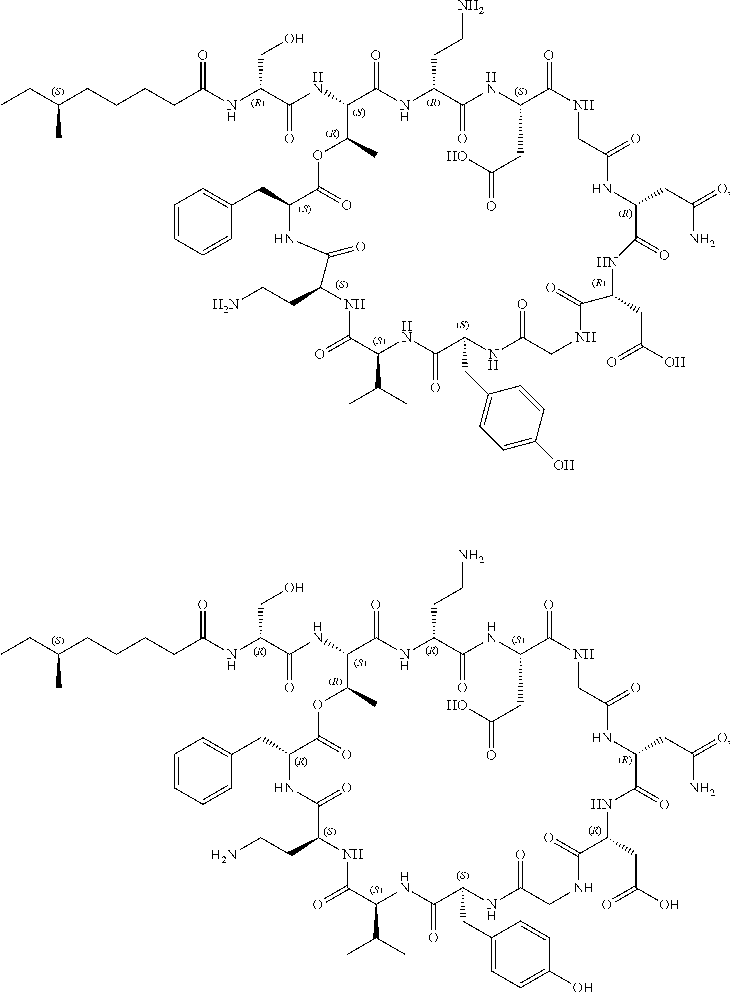

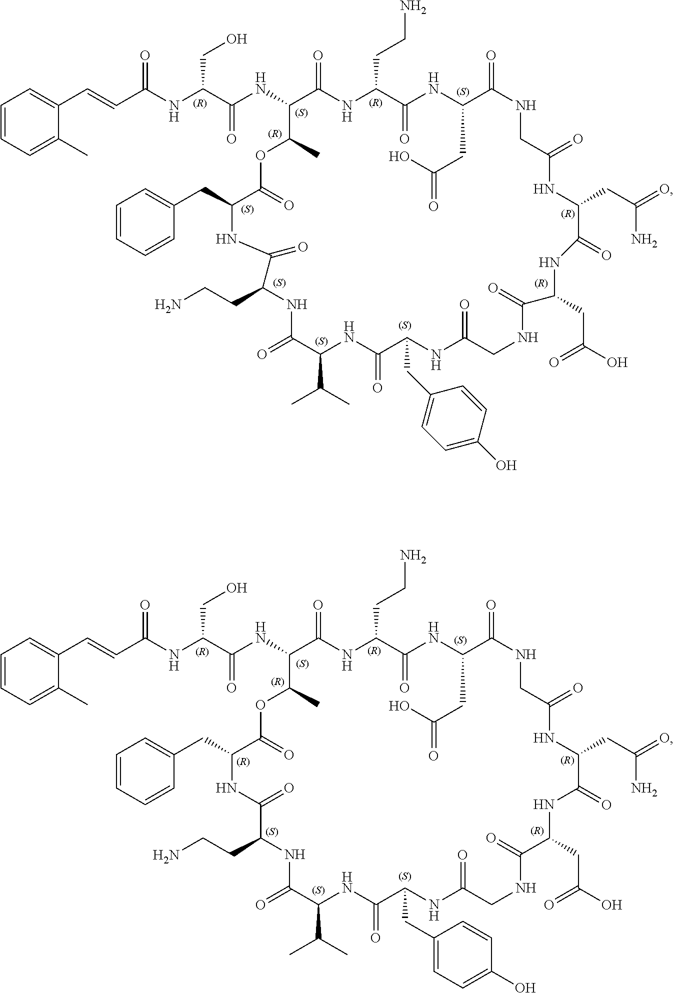

or a racemate, an enantiomer, a diastereomer, a pharmaceutically acceptable salt, or a derivative thereof.

The compounds described herein may form salts with acids or bases, and such salts are included in the present invention. The term “salts” embraces addition salts of free acids or free bases that are compounds of the invention.

In one aspect, the present invention relates, in part, to compositions comprising one or more compounds of the present invention. In some embodiments, the composition comprises one or more compounds having the structure of Formulae (I)-(IV), or a racemate, an enantiomer, a diastereomer, a pharmaceutically acceptable salt, or a derivative thereof. In some embodiments, the composition is the pharmaceutical composition.

### Methods of Generating Compounds

In one aspect, the present invention relates, in part, to a method of generating one or more compounds of the present invention. In various embodiments, the compounds of the present invention can be generated using any method known to those of skill in the art. For example, in one embodiment, the compounds can be synthesized using any method known to those of skill in the art. For example, the compounds of the present invention may be synthesized using techniques well-known in the art of organic synthesis. The starting materials and intermediates required for the synthesis may be obtained from commercial sources or synthesized according to methods known to those skilled in the art.

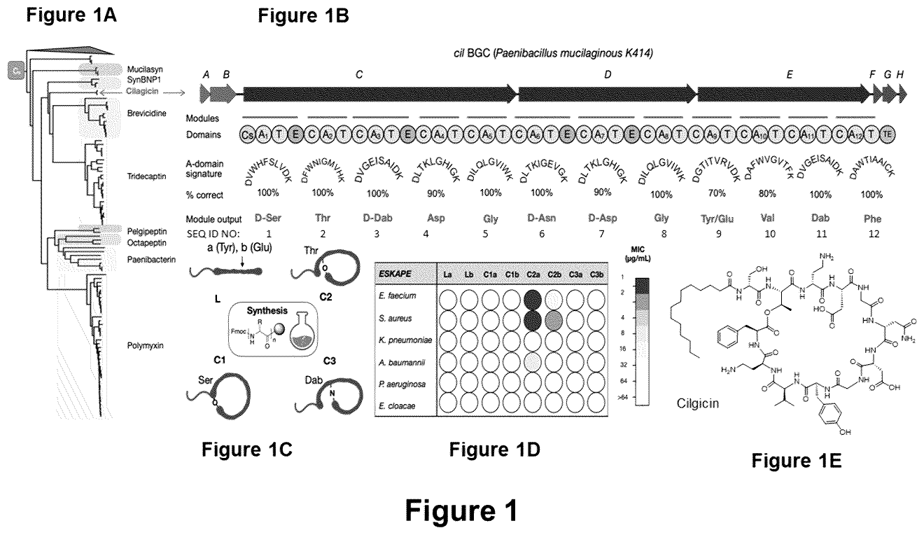

In one embodiment, the present invention provides methods of generating the compounds of the present invention via isolated nucleic acids encoding the compound of the present invention. In one embodiment, when the nucleic acids are administered to a subject, they produce the compound of the present invention. In one embodiment, when the nucleic acids are administered to a subject, they produce an antibacterial effect. For example, in some embodiments, the nucleic acid molecule comprises at least one nucleotide sequence of FIG. 1.

In another embodiment, the present invention provides methods of generating the compounds of the present invention via isolated nucleic acids and vectors encoding the compound of the present invention. In one embodiment, when the nucleic acids and vectors are administered to a subject, they produce the compound of the present invention. In one embodiment, when the nucleic acids and vectors are administered to a subject, they produce an antibacterial effect.

The nucleic acid sequences include both the DNA sequence that is transcribed into RNA and the RNA sequence that is translated into a polypeptide. According to other embodiments, the polynucleotides of the invention are inferred from the amino acid sequence of the polypeptides of the invention. As is known in the art several alternative polynucleotides are possible due to redundant codons, while retaining the biological activity of the translated polypeptides.

It is to be understood explicitly that the scope of the present invention encompasses homologs, analogs, variants, fragments, derivatives and salts, including shorter and longer polynucleotides as well as polynucleotide analogs with one or more nucleic acid substitution, as well as nucleic acid derivatives, non-natural nucleic acids and synthetic nucleic acids as are known in the art, with the stipulation that these modifications must preserve the activity of the original molecule. The invention should be construed to include any and all isolated nucleic acids which are homologous to the nucleic acids described and referenced herein.

The skilled artisan would understand that the nucleic acids of the invention encompass a RNA or a DNA sequence comprising a sequence of the invention, and any modified forms thereof, including chemical modifications of the DNA or RNA which render the nucleotide sequence more stable when it is cell free or when it is associated with a cell. Chemical modifications of nucleotides may also be used to enhance the efficiency with which a nucleotide sequence is taken up by a cell or the efficiency with which it is expressed in a cell. Any and all combinations of modifications of the nucleotide sequences are contemplated in the present invention.

The coding sequence may comprise a codon that may allow more efficient transcription of the coding sequence in the host cell. In one embodiment, viral vectors are provided herein which are capable of delivering a nucleic acid of the invention to a cell. The expression vector may be provided to a cell in the form of a viral vector. Viral vector technology is well known in the art and is described, for example, in Sambrook et al. (2001), and in Ausubel et al. (1997), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Suitable host organisms include microorganisms, plant cells, and plants. The microorganism can be any microorganism suitable for expression of heterologous nucleic acids. In one embodiment the host organism of the invention is a eukaryotic cell. In another embodiment the host organism is a prokaryotic cell. In one embodiment, the host organism is a fungal cell such as a yeast or filamentous fungus. In one embodiment the host organism may be a yeast cell.

The host organism may also be a plant. plant or plant cell can be transformed by having a heterologous nucleic acid integrated into its genome, i.e., it can be stably transformed. Stably transformed cells typically retain the introduced nucleic acid with each cell division. A plant or plant cell can also be transiently transformed such that the recombinant gene is not integrated into its genome. Transiently transformed cells typically lose all or some portion of the introduced nucleic acid with each cell division such that the introduced nucleic acid cannot be detected in daughter cells after a certain number of cell divisions.

In one embodiment, the engineered cell produces a compound of Formula (I). In some embodiments, the engineered cell produces at least one compound of Formula (I)-(IV). For example, in one embodiment, the engineered cell produces a compound of Formula (I). In one embodiment, the engineered cell produces a compound of Formula (II). In one embodiment, the engineered cell produces a compound of Formula (III). In one embodiment, the engineered cell produces a compound of Formula (IV).

In one embodiment, the cell is a eukaryotic cell. In one embodiment, the cell may be a human cell, a non-human mammalian cell, a non-mammalian vertebrate cell, an invertebrate cell, an insect cell, a plant cell, a yeast cell, or a single cell eukaryotic organism. In one embodiment, the cell may be an adult cell or an embryonic cell (e.g., an embryo). In one embodiment, the cell may be a stem cell. Suitable stem cells include without limit embryonic stem cells, ES-like stem cells, fetal stem cells, adult stem cells, pluripotent stem cells, induced pluripotent stem cells, multipotent stem cells, oligopotent stem cells, unipotent stem cells and others.

In one embodiment, the cell is a cell line cell. Non-limiting examples of suitable mammalian cells include Chinese hamster ovary (CHO) cells, baby hamster kidney (BHK) cells; mouse myeloma NSO cells, mouse embryonic fibroblast 3T3 cells (NIH3T3), mouse B lymphoma A20 cells; mouse melanoma B16 cells; mouse myoblast C₂C12 cells; mouse myeloma SP2/0 cells; mouse embryonic mesenchymal C3H-TOTT/2 cells; mouse carcinoma CT26 cells, mouse prostate DuCuP cells; mouse breast EMT6 cells; mouse hepatoma Hepalclc7 cells; mouse myeloma J5582 cells; mouse epithelial MTD-TA cells; mouse myocardial MyEnd cells; mouse renal RenCa cells; mouse pancreatic RIN-5F cells; mouse melanoma X64 cells; mouse lymphoma YAC-1 cells; rat glioblastoma 9L cells; rat B lymphoma RBL cells; rat neuroblastoma B35 cells; rat hepatoma cells (HTC); buffalo rat liver BRL 3A cells; canine kidney cells (MDCK); canine mammary (CMT) cells; rat osteosarcoma D17 cells; rat monocyte/macrophage DH82 cells; monkey kidney SV-40 transformed fibroblast (COS7) cells; monkey kidney CVI-76 cells; African green monkey kidney (VERO-76) cells; human embryonic kidney cells (HEK293, HEK293T); human cervical carcinoma cells (HELA); human lung cells (W138); human liver cells (Hep G2); human U2-OS osteosarcoma cells, human A549 cells, human A-431 cells, human SW48 cells, human HCT116 cells, and human K562 cells. An extensive list of mammalian cell lines may be found in the American Type Culture Collection catalog (ATCC, Manassas, Va.).

In one embodiment, the cell can be a prokaryotic cell or a eukaryotic cell. In one embodiment, the cell is a prokaryotic cell. In one embodiment, the cell is a genetically engineered bacteria cell.

In one embodiment, the genetically engineered bacteria cell is a non-pathogenic bacteria cell. In some embodiments, the genetically engineered bacteria cell is a commensal bacteria cell. In some embodiments, the genetically engineered bacteria cell is a probiotic bacteria cell. In some embodiments, the genetically engineered bacteria cell is a naturally pathogenic bacteria cell that is modified or mutated to reduce or eliminate pathogenicity.

Exemplary bacteria include, but are not limited to Acinetobacter baumannii, Bacillus, Bacteroides, Bifidobacterium, Brevibacteria, Clostridium, Enterococcus, Escherichia coli, Lactobacillus, Lactococcus, Saccharomyces, and Staphylococcus, e.g., Bacillus coagulans, Bacillus subtilis, Bacteroides fragilis, Bacteroides subtilis, Bacteroides thetaiotaomicron, Bifidobacterium bifidum, Bifidobacterium infantis, Bifidobacterium lactis, Bifidobacterium longum, Clostridium butyricum, Clostridium difficile, Enterobacter species, Enterobacter cloacae, Enterococcus casseliflavus, Enterococcus faecium, Enterococcus gallinarum, Escherichia coli, Klebsiella pneumoniae, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, Pseudomonas aeruginosa, Saccharomyces boulardii, Staphylococcus aureus, Streptococcus agalactiae, Streptococcus pneumoniae, and Streptococcus pyrogens.

In some embodiments, the genetically engineered bacteria are Escherichia coli strain Nissle 1917 (E. coli Nissle), a Gram-negative bacterium of the Enterobacteriaceae family that “has evolved into one of the best characterized probiotics” (Ukena et al., 2007). The strain is characterized by its complete harmlessness (Schultz, 2008), and has GRAS (generally recognized as safe) status (Reister et al., 2014, emphasis added). Genomic sequencing confirmed that E. coli Nissle lacks prominent virulence factors (e.g., E. coli α-hemolysin, P-fimbrial adhesins) (Schultz, 2008). In addition, it has been shown that E. coli Nissle does not carry pathogenic adhesion factors, does not produce any enterotoxins or cytotoxins, is not invasive, and not uropathogenic (Sonnenborn et al., 2009). As early as in 1917, E. coli Nissle was packaged into medicinal capsules, called Mutaflor, for therapeutic use. E. coli Nissle has since been used to treat ulcerative colitis in humans in vivo (Rembacken et al., 1999), to treat inflammatory bowel disease, Crohn's disease, and pouchitis in humans in vivo (Schultz, 2008), and to inhibit enteroinvasive Salmonella, Legionella, Yersinia, and Shigella in vitro (Altenhoefer et al., 2004). It is commonly accepted that E. coli Nissle's therapeutic efficacy and safety have convincingly been proven (Ukena et al., 2007).

One of ordinary skill in the art would appreciate that the genetic modifications disclosed herein may be modified and adapted for other species, strains, and subtypes of bacteria.

### Treatment Methods

In one aspect, the invention provides methods of treating or preventing an infection in a subject in need thereof. In some embodiments, the method comprises administering to the subject an effective amount of a composition comprising at least one compound of the invention (e.g., at least one compound of Formula (I)-(IV)). In some embodiments, the method comprises administering to the subject an effective amount of a composition comprising at least one nucleic acid of the invention.

In another aspect, the invention provides methods of administering an effective amount of a composition comprising at least one compound of the invention (e.g., at least one compound of Formula (I)-(IV)) to a subject. In some embodiments, the method comprises administering to the subject an effective amount of a composition comprising at least one nucleic acid of the invention. In some embodiments, the subject has an infection.

In some embodiments, the method treats or prevents a bacterial infection. In one embodiment, the method treats or prevents a gram-positive bacterial infection. In one embodiment, the bacterial infection is resistant to antibiotics. For example, in one embodiment, the bacterial infection is resistant to one or more of, beta-lactams, including methicillin, oxacillin, or penicillin, tetracyclines, gentamicin, kanamycin, erythromycin, spectinomycin, and vancomycin.

Exemplary bacterial infections that may be treated by way of the present invention includes, but is not limited to, infections caused by bacteria from the taxonomic genus of Acinetobacter, Bacillus, Bartonella, Bordetella, Borrelia, Brucella, Campylobacter, Chlamydia, Chlamydophila, Clostridium, Corynebacterium, Enterobacter, Enterococcus, Escherichia, Francisella, Haemophilus, Helicobacter, Klebsiella, Legionella, Leptospira, Listeria, Mycobacterium, Mycoplasma, Neisseria, Pseudomonas, Rickettsia, Salmonella, Shigella, Staphylococcus, Streptococcus, Treponema, Ureaplasma, Vibrio, and Yersinia. In some embodiments, the bacterial infection is an infection of Acinetobacter baumannii, Bacillus anthracis, Bacillus cereus, Bacillus subtilis, Bartonella henselae, Bartonella quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus casseliflavus, Enterobacter cloacae, Enterococcus faecalis, Enterococcus faecium, Enterococcus gallinarum, Escherichia coli, Francisella tularensis, Haemophilus influenzae, Helicobacter pylori, Klebsiella pneumoniae, Legionella pneumophila, Leptospira interrogans, Leptospira santarosai, Leptospira weilii, Leptospira noguchii, Listeria monocytogenes, Morexella species, Moraxella osloensis, Mycobacterium leprae, Mycobacterium tuberculosis, Mycobacterium ulcerans, Mycoplasma pneumoniae, Neisseria gonorrhoeae, Neisseria meningitidis, Proteus species, Proteus vulgaris, Pseudomonas aeruginosa, Rickettsia rickettsii, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Ureaplasma urealyticum, Vibrio cholerae, Yersinia pestis, Yersinia enterocolitica, or Yersinia pseudotuberculosis. In one embodiment, the bacterial infection is a Listeria monocytogenes infection.

In one embodiment, the bacterial infection is an infection of S. aureus USA300, S. aureus COL, S. aureus BAA-42, S. aureus NRS100, S. aureus NRS108, S. aureus NRS140, S. aureus NRS146, E. faecium VRE, E. faecium Com15, S. pneumoniae, S. mutans, B. subtilis, L. rhamnosus, E. coli, C. albicans, or C. neoformans.

Exemplary diseases caused by bacterial infections which may be treated using compositions of the present invention, include but are not limited to, bacterially mediated meningitis, sinus tract infections, pneumonia, endocarditis, pancreatitis, appendicitis, gastroenteritis, biliary tract infections, soft tissue infections, urinary tract infections, cystitis, pyelonephritis, osteomyelitis, bacteremia, Actinomycosis, Whooping cough, Secondary bacterial pneumonia, Lyme disease (B. burgdorferi), Relapsing fever, Brucellosis, Enteritis, bloody diarrhea, Guillain-Barre syndrome, Atypical pneumonia, Trachoma, Neonatal conjunctivitis, Neonatal pneumonia, Nongonococcal urethritis(NGU), Urethritis, Pelvic inflammatory disease, Epididymitis, Prostatitis, Lymphogranuloma venereum (LGV), Psittacosis, Botulism: Mainly muscle weakness and paralysis, Pseudomembranous colitis, Anaerobic cellulitis, Gas gangrene Acutefood poisoning, Tetanus, and Diphtheria.

However, the invention should not be limited to only treating bacterial infection. The invention encompasses compounds having an antimicrobial activity including but not limited to antibacterial, antimycobacterial, antifungal, antiviral and the likes.

In one aspect, the invention provides methods of killing a bacterial cell or inhibiting the grown of a bacterial cell. In some embodiments, the method comprises administering to the cell an effective amount of a composition comprising at least one compound of the invention. In some embodiments, the method comprises administering to the cell an effective amount of a composition comprising at least one nucleic acid of the invention. In one embodiment the bacterial cell is a gram positive bacterial cell. In one embodiment, the bacterial cell is resistant to antibiotics. For example, in one embodiment, the bacterial cell is resistant to one or more of, beta-lactams, including methicillin, oxacillin, or penicillin, tetracyclines, gentamicin, kanamycin, erythromycin, spectinomycin, and vancomycin.

In another aspect, the invention provides compositions and methods for treating and/or preventing a disease or disorder related to the detrimental growth and/or proliferation of a bacterial cell in vivo, ex vivo or in vitro. In certain embodiments, the method comprises administering a composition comprising an effective amount of a composition provided by the invention to a subject, wherein the composition is effective in inhibiting or preventing the growth and/or proliferation of a bacterial cell. In certain embodiments, the bacterial cell is a Gram-positive bacterial cell, e.g., a bacteria of a genera such as Staphylococcus, Streptococcus, Enterococcus, (which are cocci) and Bacillus, Corynebacterium, Nocardia, Clostridium, Actinobacteria, and Listeria (which are rods and can be remembered by the mnemonic obconical), Mollicutes, bacteria-like Mycoplasma, Actinobacteria.

In certain embodiments, the bacterial cell is a Gram− bacteria cell, e.g., a bacteria of a genera such as Acinetobacter, Citrobacter, Enterobacter, Enterococcus, Escherichia, Helicobacter, Hemophilus, Klebsiella, Legionella, Moraxella, Neisseria, Proteus, Pseudomonas, Salmonella, Staphylococcus, and Yersinia. The compounds as described herein and compositions comprising them may thus be for use in the treatment of bacterial infections by the above-mentioned Gram+ or Gram− bacteria.

In one embodiment, the method further comprises administering a second therapeutic agent. In one embodiment, the second therapeutic agent is an antibiotic agent. In one embodiment, the compound of the invention and the at least one additional antibiotic agent act synergistically in preventing, reducing or disrupting microbial growth.

Non-limiting examples of the at least one additional antibiotic agents include levofloxacin, doxycycline, neomycin, clindamycin, minocycline, gentamycin, rifampin, chlorhexidine, chloroxylenol, methylisothizolone, thymol, α-terpineol, cetylpyridinium chloride, hexachlorophene, triclosan, nitrofurantoin, erythromycin, nafcillin, cefazolin, imipenem, astreonam, gentamicin, sulfamethoxazole, vancomycin, ciprofloxacin, trimethoprim, rifampin, metronidazole, clindamycin, teicoplanin, mupirocin, azithromycin, clarithromycin, ofoxacin, lomefloxacin, norfloxacin, nalidixic acid, sparfloxacin, pefloxacin, amifloxacin, gatifloxacin, moxifloxacin, gemifloxacin, enoxacin, fleroxacin, minocycline, linexolid, temafloxacin, tosufloxacin, clinafloxacin, sulbactam, clavulanic acid, amphotericin B, fluconazole, itraconazole, ketoconazole, nystatin, penicillins, cephalosporins, carbepenems, beta-lactams antibiotics, aminoglycosides, macrolides, lincosamides, glycopeptides, tetracylines, chloramphenicol, quinolones, fucidines, sulfonamides, trimethoprims, rifamycins, oxalines, streptogramins, lipopeptides, ketolides, polyenes, azoles, echinocandines, and any combination thereof.

In one embodiment, the compositions of the invention find use in removing at least a portion of or reducing the number of microorganisms and/or biofilm-embedded microorganisms attached to the surface of a medical device or the surface of a subject's body (such as the skin of the subject, or a mucous membrane of the subject, such as the vagina, anus, throat, eyes or ears). In one embodiment, the compositions of the invention find further use in coating the surface of a medical device, thus inhibiting or disrupting microbial growth and/or inhibiting or disrupting the formation of biofilm on the surface of the medical device. The compositions of the invention find further use in preventing or reducing the growth or proliferation of microorganisms and/or biofilm-embedded microorganisms on the surface of a medical device or on the surface of a subject's body. However, the invention is not limited to applications in the medical field. Rather, the invention includes using a compound or an analog thereof as an antimicrobial and/or antibiofilm agent in any setting.

The composition of the invention may be administered to a patient or subject in need in a wide variety of ways, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous (i.v.) injection, or intraperitoneally. In one embodiment, the composition is administered systemically to the subject. In one embodiment, the compositions of the present invention are administered to a patient by i.v. injection. In one embodiment, the composition is administered locally to the subject. In one embodiment, the compositions of the present invention are administered to a patient topically. Any administration may be a single application of a composition of invention or multiple applications. Administrations may be to single site or to more than one site in the individual to be treated. Multiple administrations may occur essentially at the same time or separated in time.

In one aspect, the compositions of the invention may be in the form of a coating that is applied to the surface of a medical device or the surface of a subject's body. In one embodiment, the coating prevents or hinders microorganisms and/or biofilm-embedded microorganisms from growing and proliferating on at least one surface of the medical device or at least one surface of the subject's body. In another embodiment, the coating facilitates access of antimicrobial agents to the microorganisms and/or biofilm-embedded microorganisms, thus helping prevent or hinder the microorganisms and/or biofilm-embedded microorganisms from growing or proliferating on at least one surface of the medical device or at least one surface of the subject's body. The compositions of the invention may also be in the form of a liquid or solution, used to clean the surface of medical device or the surface of a subject's body, on which microorganisms and/or biofilm-embedded microorganisms live and proliferate. Such cleaning of the medical device or body surface may occur by flushing, rinsing, soaking, or any additional cleaning method known to those skilled in the art, thus removing at least a portion of or reducing the number of microorganisms and/or biofilm-embedded microorganisms attached to at least one surface of the medical device or at least one surface of the subject's body.

Subjects to which administration of the pharmaceutical compositions of the invention is contemplated include, but are not limited to, humans and other primates, mammals including but not limited to non-human mammals such as non-human primates, cattle, pigs, horses, sheep, cats, and dogs.

Pharmaceutical compositions of the present invention may be administered in a manner appropriate to the disease to be treated (or prevented). The quantity and frequency of administration will be determined by such factors as the condition of the subject, and the type and severity of the subject's disease, although appropriate dosages may be determined by clinical trials.

When “therapeutic amount” is indicated, the precise amount of the compositions of the present invention to be administered can be determined by a physician with consideration of individual differences in age, weight, disease type, extent of disease, and condition of the patient (subject).

### Dosage and Formulation (Pharmaceutical Compositions)

The invention also encompasses the use of pharmaceutical compositions comprising a compound of the invention, a nucleic acid of the invention, or salts thereof. Such a pharmaceutical composition may comprise of at least one a compound of the invention, a nucleic acid of the invention, or salts thereof in a form suitable for administration to a subject, or the pharmaceutical composition may comprise at least one a compound of the invention, a nucleic acid of the invention, or salts thereof, and one or more pharmaceutically acceptable carriers, one or more additional ingredients, or some combination of these. The compound or nucleic acid of the invention may be present in the pharmaceutical composition in the form of a physiologically acceptable salt, such as in combination with a physiologically acceptable cation or anion, as is well known in the art.

Administration of the therapeutic agent in accordance with the present invention may be continuous or intermittent, depending, for example, upon the recipient's physiological condition, whether the purpose of the administration is therapeutic or prophylactic, and other factors known to skilled practitioners. The administration of the agents of the invention may be essentially continuous over a preselected period of time or may be in a series of spaced doses. Both local and systemic administration is contemplated. The amount administered will vary depending on various factors including, but not limited to, the composition chosen, the particular disease, the weight, the physical condition, and the age of the subject, and whether prevention or treatment is to be achieved. Such factors can be readily determined by the clinician employing animal models or other test systems which are well known to the art

The formulations may, where appropriate, be conveniently presented in discrete unit dosage forms and may be prepared by any of the methods well known to pharmacy. Such methods may include the step of bringing into association the therapeutic agent with liquid carriers, solid matrices, semi-solid carriers, finely divided solid carriers or combinations thereof, and then, if necessary, introducing or shaping the product into the desired delivery system.

In one embodiment, the pharmaceutical compositions useful for practicing the methods of the invention may be administered to deliver a dose of between 1 ng/kg/day and 100 mg/kg/day. In another embodiment, the pharmaceutical compositions useful for practicing the invention may be administered to deliver a dose of between 1 ng/kg/day and 500 mg/kg/day.

Typically, dosages which may be administered in a method of the invention to a mammal, preferably a human, range in amount from 0.5 μg to about 50 mg per kilogram of body weight of the mammal, while the precise dosage administered will vary depending upon any number of factors, including but not limited to, the type of mammal and type of disease state being treated, the age of the mammal and the route of administration. Preferably, the dosage of the compound will vary from about 1 μg to about 10 mg per kilogram of body weight of the mammal. More preferably, the dosage will vary from about 3 μg to about 5 mg per kilogram of body weight of the mammal.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

The composition may be administered to a mammal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the mammal, etc.

When the therapeutic agents of the invention are prepared for administration, they are preferably combined with a pharmaceutically acceptable carrier, diluent or excipient to form a pharmaceutical formulation, or unit dosage form. The total active ingredients in such formulations include from 0.1 to 99.9% by weight of the formulation. A “pharmaceutically acceptable” is a carrier, diluent, excipient, and/or salt that is compatible with the other ingredients of the formulation, and not deleterious to the recipient thereof. The active ingredient for administration may be present as a powder or as granules; as a solution, a suspension or an emulsion.

Pharmaceutical formulations containing the therapeutic agents of the invention can be prepared by procedures known in the art using well known and readily available ingredients. The therapeutic agents of the invention can also be formulated as solutions appropriate for parenteral administration, for instance by intramuscular, subcutaneous or intravenous routes.

The pharmaceutical formulations of the therapeutic agents of the invention can also take the form of an aqueous or anhydrous solution or dispersion, or alternatively the form of an emulsion or suspension.

Thus, the therapeutic agent may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

It will be appreciated that the unit content of active ingredient or ingredients contained in an individual aerosol dose of each dosage form need not in itself constitute an effective amount for treating the particular indication or disease since the necessary effective amount can be reached by administration of a plurality of dosage units. Moreover, the effective amount may be achieved using less than the dose in the dosage form, either individually, or in a series of administrations.

The pharmaceutical formulations of the present invention may include, as optional ingredients, pharmaceutically acceptable carriers, diluents, solubilizing or emulsifying agents, and salts of the type that are well-known in the art. Specific non-limiting examples of the carriers and/or diluents that are useful in the pharmaceutical formulations of the present invention include water and physiologically acceptable buffered saline solutions, such as phosphate buffered saline solutions pH 7.0-8.0.

The compounds and polypeptides (active ingredients) of this invention can be formulated and administered to treat a variety of disease states by any means that produces contact of the active ingredient with the agent's site of action in the body of the organism.

They can be administered by any conventional means available for use in conjunction with pharmaceuticals, either as individual therapeutic active ingredients or in a combination of therapeutic active ingredients. They can be administered alone, but are generally administered with a pharmaceutical carrier selected on the basis of the chosen route of administration and standard pharmaceutical practice.

In general, water, suitable oil, saline, aqueous dextrose (glucose), and related sugar solutions and glycols such as propylene glycol or polyethylene glycols are suitable carriers for parenteral solutions. Solutions for parenteral administration contain the active ingredient, suitable stabilizing agents and, if necessary, buffer substances. Antioxidizing agents such as sodium bisulfate, sodium sulfite or ascorbic acid, either alone or combined, are suitable stabilizing agents. Also used are citric acid and its salts and sodium Ethylenediaminetetraacetic acid (EDTA). In addition, parenteral solutions can contain preservatives such as benzalkonium chloride, methyl- or propyl-paraben and chlorobutanol. Suitable pharmaceutical carriers are described in Remington's Pharmaceutical Sciences, a standard reference text in this field.

The active ingredients of the invention may be formulated to be suspended in a pharmaceutically acceptable composition suitable for use in mammals and in particular, in humans. Such formulations include the use of adjuvants such as muramyl dipeptide derivatives (MDP) or analogs that are described in U.S. Pat. Nos. 4,082,735; 4,082,736; 4,101,536; 4,185,089; 4,235,771; and 4,406,890. Other adjuvants, which are useful, include alum (Pierce Chemical Co.), lipid A, trehalose dimycolate and dimethyldioctadecylammonium bromide (DDA), Freund's adjuvant, and IL-12. Other components may include a polyoxypropylene-polyoxyethylene block polymer (Pluronic®), a non-ionic surfactant, and a metabolizable oil such as squalene (U.S. Pat. No. 4,606,918).

Additionally, standard pharmaceutical methods can be employed to control the duration of action. These are well known in the art and include control release preparations and can include appropriate macromolecules, for example polymers, polyesters, polyamino acids, polyvinyl, pyrolidone, ethylenevinylacetate, methyl cellulose, carboxymethyl cellulose or protamine sulfate. The concentration of macromolecules as well as the methods of incorporation can be adjusted in order to control release. Additionally, the agent can be incorporated into particles of polymeric materials such as polyesters, polyamino acids, hydrogels, poly (lactic acid) or ethylenevinylacetate copolymers. In addition to being incorporated, these agents can also be used to trap the compound in microcapsules.

Accordingly, the pharmaceutical composition of the present invention may be delivered via various routes and to various sites in a mammal body to achieve a particular effect (see, e.g., Rosenfeld et al., 1991; Rosenfeld et al., 1991a; Jaffe et al., supra; Berkner, supra). One skilled in the art will recognize that although more than one route can be used for administration, a particular route can provide a more immediate and more effective reaction than another route. Local or systemic delivery can be accomplished by administration comprising application or instillation of the formulation into body cavities, inhalation or insufflation of an aerosol, or by parenteral introduction, comprising intramuscular, intravenous, peritoneal, subcutaneous, intradermal, as well as topical administration.

The active ingredients of the present invention can be provided in unit dosage form wherein each dosage unit, e.g., a teaspoonful, tablet, solution, or suppository, contains a predetermined amount of the composition, alone or in appropriate combination with other active agents. The term “unit dosage form” as used herein refers to physically discrete units suitable as unitary dosages for human and mammal subjects, each unit containing a predetermined quantity of the compositions of the present invention, alone or in combination with other active agents, calculated in an amount sufficient to produce the desired effect, in association with a pharmaceutically acceptable diluent, carrier, or vehicle, where appropriate. The specifications for the unit dosage forms of the present invention depend on the particular effect to be achieved and the particular pharmacodynamics associated with the pharmaceutical composition in the particular host.

In one embodiment, the compositions of the invention are formulated using one or more pharmaceutically acceptable excipients or carriers. In one embodiment, the pharmaceutical compositions of the invention comprise a therapeutically effective amount of a compound or conjugate of the invention and a pharmaceutically acceptable carrier.

Pharmaceutically acceptable carriers that are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions may be brought about by including in the composition an agent that delays absorption, for example, aluminum monostearate or gelatin. In one embodiment, the pharmaceutically acceptable carrier is not DMSO alone.

The present invention also provides pharmaceutical compositions comprising one or more of the compositions described herein. Formulations may be employed in admixtures with conventional excipients, i.e., pharmaceutically acceptable organic or inorganic carrier substances suitable for administration to subject. The pharmaceutical compositions may be sterilized and if desired mixed with auxiliary agents, e.g., lubricants, preservatives, stabilizers, wetting agents, emulsifiers, salts for influencing osmotic pressure buffers, coloring, and/or aromatic substances and the like. They may also be combined where desired with other active agents, e.g., other analgesic agents.

As used herein, “additional ingredients” include, but are not limited to, one or more of the following: excipients; surface active agents; dispersing agents; inert diluents; granulating and disintegrating agents; binding agents; lubricating agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the invention are known in the art and described, for example in Genaro, ed. (1985, Remington's Pharmaceutical Sciences, Mack Publishing Co., Easton, PA), which is incorporated herein by reference.

The composition of the invention may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

In an embodiment, the composition includes an anti-oxidant and a chelating agent that inhibits the degradation of one or more components of the composition. Preferred antioxidants for some compounds are BHT, BHA, alpha-tocopherol and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition that may be detrimental to the shelf life of the formulation. While BHT and disodium edetate are the particularly preferred antioxidant and chelating agent respectively for some compounds, other suitable and equivalent antioxidants and chelating agents may be substituted therefore as would be known to those skilled in the art.

Liquid suspensions may be prepared using conventional methods to achieve suspension of the HMW-HA or other composition of the invention in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl-para-hydroxybenzoates, ascorbic acid, and sorbic acid.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or Arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

The regimen of administration may affect what constitutes an effective amount. The therapeutic formulations may be administered to the subject either prior to or after a diagnosis of disease. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a subject, preferably a mammal, more preferably a human, may be carried out using known procedures, at dosages and for periods of time effective to prevent or treat disease. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the subject being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

The compound may be administered to a subject as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose will be readily apparent to the skilled artisan and will depend upon any number of factors, such as, but not limited to, the type and severity of the disease being treated, the type and age of the animal, etc.

Actual dosage levels of the active ingredients in the pharmaceutical compositions of this invention may be varied so as to obtain an amount of the active ingredient that is effective to achieve the desired therapeutic response for a particular subject, composition, and mode of administration, without being toxic to the subject.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease in a subject.

In one embodiment, the compositions of the invention are administered to the subject in dosages that range from one to five times per day or more. In another embodiment, the compositions of the invention are administered to the subject in range of dosages that include, but are not limited to, once every day, every two, days, every three days to once a week, and once every two weeks. It will be readily apparent to one skilled in the art that the frequency of administration of the various combination compositions of the invention will vary from subject to subject depending on many factors including, but not limited to, age, disease or disorder to be treated, gender, overall health, and other factors. Thus, the invention should not be construed to be limited to any particular dosage regime and the precise dosage and composition to be administered to any subject will be determined by the attending physical taking all other factors about the subject into account.

Compounds of the invention for administration may be in the range of from about 1 mg to about 10,000 mg, about 20 mg to about 9,500 mg, about 40 mg to about 9,000 mg, about 75 mg to about 8,500 mg, about 150 mg to about 7,500 mg, about 200 mg to about 7,000 mg, about 3050 mg to about 6,000 mg, about 500 mg to about 5,000 mg, about 750 mg to about 4,000 mg, about 1 mg to about 3,000 mg, about 10 mg to about 2,500 mg, about 20 mg to about 2,000 mg, about 25 mg to about 1,500 mg, about 50 mg to about 1,000 mg, about 75 mg to about 900 mg, about 100 mg to about 800 mg, about 250 mg to about 750 mg, about 300 mg to about 600 mg, about 400 mg to about 500 mg, and any and all whole or partial increments there between.

In some embodiments, the dose of a compound of the invention is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound (i.e., a drug used for treating the same or another disease as that treated by the compositions of the invention) as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In one embodiment, the present invention is directed to a packaged pharmaceutical composition comprising a container holding a therapeutically effective amount of a compound or conjugate of the invention, alone or in combination with a second pharmaceutical agent; and instructions for using the compound or conjugate to treat, prevent, or reduce one or more symptoms of a disease in a subject.

The term “container” includes any receptacle for holding the pharmaceutical composition. For example, in one embodiment, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating or preventing a disease in a subject, or delivering an imaging or diagnostic agent to a subject.

Routes of administration of any of the compositions of the invention include oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

These methods described herein are by no means all-inclusive, and further methods to suit the specific application will be apparent to the ordinary skilled artisan. Moreover, the effective amount of the compositions can be further approximated through analogy to compounds known to exert the desired effect.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1: Cilagicins, Bifunctional Antibiotics that Evade Resistance

The present studies demonstrated the use of a phylogenetic analysis of condensation starter domain (Cs) sequences, which introduced the acyl substituent into lipopeptides, to identify the cryptic cil biosynthetic gene cluster (BGC) as a potential source of an uncharacterized lipopeptide antibiotic. Detailed bioinformatic analysis of this BGC inspired the chemical synthesis of the antibiotic cilagicin. Cilagicin's unprecedented ability to inhibit cell wall biosynthesis by sequestering both the glycan lipid carrier undecaprenyl phosphate (C55-P) and its precursor undecaprenyl pyrophosphate (C55-PP), likely explains the absence of any observed resistance in laboratory experiments or among multi-drug resistant clinical isolates.

An optimized biphenyl analog of cilagicin, cilagicin BP, is active against multi-drug resistant (MDR) Gram-positive pathogens both in vitro and in vivo. Its in vivo efficacy, combined with an absence of detectable resistance makes cilagicin BP an exciting candidate for developing therapies that can address infections caused by multiple MDR pathogens.

### Identification of the Cil BGC

To identify BGCs that encode lipopeptide antibiotics with new MOAs, NRPS BGCs were collected from ~10,000 sequenced bacterial genomes. Clinically relevant lipopeptide antibiotics (e.g., polymyxin, daptomycin, etc.) have historically tended to be larger macrocyclic structures, and therefore BGCs encoding peptides with fewer than 5 amino acids (i.e., containing less than 5 adenylation domains) were removed from this collection. The key conserved feature across lipopeptides is the presence of an N-terminal lipid that is installed by a Cs domain (Bloudoff et al., 2017, Biochim Biophys Acta Proteins Proteom, 1865:1587-1604; Rausch et al., 2007, BMC Evol Biol, 7:78; Roongsawang et al., 2005, FEMS Microbiol Lett, 252:143-151).

Among the sequenced large NRPS BGCs that were collected, 3,426 were identified to contain a Cs domain. Cs domain sequences from these BGCs were used to construct a phylogenetic tree that guided the present discovery efforts. As seen with other biosynthetic genes, sequences arising from BGCs sharing close common ancestors and hence the same MOA are likely to group together into the same clade (Kang et al., 2014, ACS Chem Biol, 9:1267-1272; Hover et al., 2018, Nat Microbiol, 3:415-422; Wang et al., 2021, Angewandte Chemie, 133:22346-22351). By extension, clades that do not contain any sequences from characterized BGCs represent candidates for identifying structurally and mechanistically new antibiotics.

The Cs domain phylogenetic tree contained a number of clades that were not associated with any characterized lipopeptides; however, one was particularly intriguing as it fell into a larger group of sequences where most other clades were associated with antibiotic biosynthesis. These included BGCs for a number of clinically used, as well as clinically appealing, antibiotics (e.g., polymyxins, tridecaptins and brevicidines). This “orphan” Cs clade that were identified contained three closely related sequences that arose from the same BGC found in two different sequenced Paenibacillus mucilaginosus strains (KNP414 and K02) (FIG. 1A). Based on gene content and gene organization, this BGC, which was labelled as the cil BGC, did not resemble any characterized BGCs and became the focus of the study outlined here.

### Structures Encoded by the Cil BGC

The vast majority of sequenced BGCs remain silent in the laboratory, even when interrogated with advanced synthetic biology tools (Tomm et al., 2019, J Ind Microbiol Biotechnol, 46:1381-1400). Existing in vivo biological approaches for accessing natural products from large BGCs will, for the foreseeable future, likely leave many BGCs functionally inaccessible. Alternative approaches for converting the genetic information in sequenced BGCs into novel bioactive small molecules are therefore needed. With the power of modern synthetic organic chemistry and the increasing accuracy of natural product structure prediction algorithms, it is now possible to generate a bioactive molecule from the genetic instructions found in the primary sequence of a BGC by first bioinformatically generating the encoded structure and then chemically synthesizing the generated structure (i.e., produce a synthetic-bioinformatic natural product, syn-BNP) (Chu et al., 2016, Nat Chem Biol, 12:1004-1006; Chu et al., 2020, J Am Chem Soc, 142:14158-14168; Chu et al., 2019, J Am Chem Soc, 141:15737-15741). In this study, a syn-BNP approach was used to generate lipopeptide structures based on the cil BGC and then tested these small molecules for antibacterial activity.

The cil BGC contained three NRPS open reading frames (cil C-E) that encode 12 distinct modules (FIG. 1B, Table 1). The biosynthesis of a 12-mer lipopeptide begins with the Cs domain at the N-terminus of CilC and end with the thioesterase at the C-terminus of CilE. The composition of each module's A-domain substrate binding pocket (i.e., substrate signature based on positions 235, 236, 239, 278, 299, 301, 322, 330, 331, 517 of the A-domain) was used to generate the 12 monomers used by this BGC (Stachelhaus et al., 1999, Chem Biol, 6:493-505).

TABLE 1
Cil biosynthetic gene cluster gene annotations (GenBank Accession Number:
NC_015690.1 location 3,145,670-3,197,723)

| | Gene size | Gene | | Protein, (Source Organism), |
|---|---|---|---|---|
| ORF | (bp) | name | Proposed function | Accession number |
| 1 | 762 | cilA | Transporter | ABC transporter ATP-binding protein |
| | | | | (Paenibacillus mucilaginosus), |
| | | | | WP_187296748.1 |
| 2 | 1929 | cilB | Transporter | FtsX-like permease family protein |
| | | | | (Paenibacillus mucilaginosus), |
| | | | | WP_013916735.1 |
| 3 | 20117 | cilC | Biosynthesis gene | non-ribosomal peptide synthetase |
| | | | | (Paenibacillus mucilaginosus) |
| 4 | 13086 | cilD | Biosynthesis gene | non-ribosomal peptide synthetase |
| | | | | (Paenibacillus mucilaginosus), |
| | | | | WP_013916739.1 |
| 5 | 12696 | cilE | Biosynthesis gene | non-ribosomal peptide synthetase |
| | | | | (Paenibacillus mucilaginosus), |
| | | | | WP_013916740.1 |
| 6 | 678 | cilF | Regulator | response regulator transcription factor |
| | | | | (Paenibacillus mucilaginosus), |
| | | | | WP_013916742.1 |
| 7 | 1062 | cilG | Regulator | sensor histidine kinase |
| | | | | (Paenibacillus mucilaginosus), |
| | | | | WP_013916743.1 |
| 8 | 594 | cilH | Regulator | TetR/AcrR family transcriptional regulator |
| | | | | (Paenibacillus mucilaginosus), |
| | | | | WP_014370159.1 |

Eleven A-domains had perfect or near perfect (80%) matches to characterized A-domains, and therefore the amino acid incorporated by these modules are determined with high confidence. The A-domain substrate signature from module 9 had equally good matches (70%) to two amino acids (Tyr and Glu). This analysis gave two potential linear lipopeptide products for the cil BGC (La and Lb) (FIG. 1C).

Epimerization domains found in modules 1, 3, 6, and 7, indicated that these amino acids appear in the D-configuration in the cil BGC product. The absence of any genes encoding tailoring enzymes (i.e., methyltransferase, hydroxylation, amino transferase, glycosyl transferase, etc.) within 10 kB of the cil NRPS genes indicated that the product of the cil BGC was not modified beyond the NRPS produced lipopeptide (Walsh et al., 2001, Current opinion in chemical biology, 5:525-534).

Naturally occurring lipopeptides appear as either linear or cyclic structures. The cil linear peptide contains three amino acids that could serve as nucleophiles (D-Ser-1, Thr-2, D-Dab-3) for cyclization through the C-terminal carboxyl. Bringing together the linear peptide and three potential cyclization sites yielded eight structures (2 linear, 6 cyclic) that could arise from the cil BGC (FIG. 1C).

Each of the eight potential BGC products was generated by solid-phase peptide synthesis (Table 2). The cil BGC did not contain any lipid biosynthetic genes and therefore the lipid found on the product of the cil BGC would likely arise directly from native fatty acid biosynthesis. Among characterized bacterial lipopeptides, myristic acid is one of the most frequently seen simple lipids and therefore all synthetic peptides were N-terminal acylated with myristic acid.

TABLE 2
High-resolution mass spectrometry (HRMS) data
for cilagicin analogs. All HRMS data were collected in positive
ionization mode with a mass range from m/z 200 to 2000.

| | | | Observed | |
|---|---|---|---|---|
| | Chemical Formula | Theoretical | Mass | error |
| Molecules | (M) | (M + H)⁺ | (M + H)⁺ | (ppm) |
| Cilagicin-La | C68H105N15O22 | 1484.7631 | 1484.7623 | 0.5 |
| Cilagicin-Lb | C64H103N15O23 | 1450.7424 | 1450.7417 | 0.5 |
| Cilagicin-C1a | C68H103N15O21 | 1466.7526 | 1466.7500 | 1.7 |
| Cilagicin-C1b | C64H101N15O22 | 1432.7318 | 1432.7330 | 0.8 |
| Cilagicin-C2a | C68H103N15O21 | 1466.7526 | 1466.7468 | 3.9 |
| Cilagicin-C2b | C64H101N15O22 | 1432.7318 | 1432.7328 | 0.7 |
| Cilagicin-C3a | C68H103N15O21 | 1466.7526 | 1466.7468 | 3.9 |
| Cilagicin-C3b | C64H101N15O22 | 1432.7318 | 1432.7311 | 0.5 |
| Cilagicin-L1 | C67H85N15O21 | 1436.6117 | 1436.6071 | 3.2 |
| Cilagicin-L2 | C74H91N15O21 | 1526.6587 | 1526.6572 | 0.9 |
| Cilagicin-L3 | C66H91N15O21 | 1430.6587 | 1430.6558 | 2.0 |
| Cilagicin-L4 | C70H88N16O21 | 1489.6383 | 1489.6383 | 1.2 |
| Cilagicin-L5 | C63H93N15O21 | 1396.6743 | 1396.6728 | 1.0 |
| Cilagicin-L6 | C64H85N15O21 | 1400.6117 | 1400.6106 | 0.8 |

All eight synthetic structures were assayed for activity against the ESKAPE pathogens (Enterococcus faecium, Staphylococcus aureus, Klebsiella pneumoniae, Acinetobacter baumannii, Pseudomonas aeruginosa, and Enterobacter species) (FIG. 1D, Table 3).

TABLE 3
Bacterial strains, human cell lines and culture conditions.

| | Name of Bacteria | Stain | Media | Culture condition |
|---|---|---|---|---|
| ESKAPE | Enterococcus faecium | Com 15 | LB | 37° C., aerobic |
| pathogens | Staphylococcus aureus | USA 300 | LB | |
| | Klebsiella pneumonia | ATCC 10031 | LB | |
| | Acinetobacter baumannii | ATCC 17978 | LB | |
| | Pseudomonas aeruginosa | PAO1 | LB | |
| | Enterobacter cloacae | ATCC 13047 | LB | |
| Antibiotic | Staphylococcus aureus | USA100 | LB | 37° C., aerobic |
| resistant | Staphylococcus aureus | USA 200 | LB | |
| S. aureus | Staphylococcus aureus | USA 500 | LB | |
| and | Staphylococcus aureus | USA 800 | LB | |
| Enterococci | Staphylococcus aureus | NRS 100 | LB | |
| strains | Staphylococcus aureus | NRS 108 | LB | |
| | Staphylococcus aureus | NRS 281 | LB | |
| | Staphylococcus aureus | NRS 22 | LB | |
| | Staphylococcus aureus | BAA-42 | LB | |
| | Staphylococcus aureus | BAA-1721 | LB | |
| | Staphylococcus aureus | BAA-1556 | LB | |
| | Staphylococcus aureus | BAA-1717 | LB | |
| | Staphylococcus aureus | NRS 146 | LB | |
| | Staphylococcus aureus | NRS 140 | LB | |
| | Staphylococcus aureus | COL (Anu 189) | LB | |
| | Staphylococcus aureus | HAR 22 | LB | |
| | Staphylococcus aureus | HU 25 | LB | |
| | Staphylococcus aureus | WCMC039967 | LB | |
| | Staphylococcus aureus | WCMC069634 | LB | |
| | Staphylococcus aureus | WCMC070502 | LB | |
| | Vancomycin-resistant Enterococci | AR 0781-0810 | LB | |
| Antibiotic | Acinetobacter baumannii | 1755 | LB | 37° C., aerobic |
| resistant | Acinetobacter baumannii | 1788 | LB | |
| A. baumannii | Acinetobacter baumannii | 1790 | LB | |
| strains | Acinetobacter baumannii | 1791 | LB | |
| | Acinetobacter baumannii | 1795 | LB | |
| | Acinetobacter baumannii | 1797 | LB | |
| | Acinetobacter baumannii | S3 | LB | |
| | Acinetobacter baumannii | S5 | LB | |
| | Clostridium difficile | HM89 | LBMª | 37° C., 5% H₂, 5% |
| | Clostridium difficile | HM746 | LBMª | CO₂, 90% N₂ |
| | Streptococcus pneumonia | R6 | LBMª | 37° C., 5% CO₂ |
| | Streptococcus pneumonia | Tigr4 | LBMª | 37° C., 5% CO₂ |
| | Streptococcus pyrogens | ATCC 19615 | LBMª | 37° C., aerobic |
| | Streptococcus agalactiae | BAA2675 | LBMª | 37° C., aerobic |
| | Streptococcus agalactiae | BAA1176 | LBMª | 37° C., aerobic |
| | Bacillus subtilis | 168A1 | LB | 37° C., aerobic |
| Human cells | Human kidney cells | HEK293 | DMEM | 37° C., 5% CO₂ |

ªLBM media was a brain heart infusion media derivate which supplemented with 5 ug/mL hemin, 1 mg/mL maltose, 1 mg/mL cellobiose and 500 ug/mL L-cysteine. Detail information can be found in previous study (Carmichael et al., 1987, Cancer Res, 47: 936-942).

The 11 amino acid macrocycle that was cyclized through the hydroxyl of Thr-2 and contained tyrosine at position 9 (compound-C₂a) showed potent antibacterial activity against the two Gram-positive ESKAPE pathogens (MIC 1 μg/mL). Interestingly, even small changes to this structure completely abrogated the antibacterial activity, as none of the remaining close analogs that were synthesized showed any significant activity against these strains. This was the active structure cilagicin (FIG. 1E). Moreover, no reported synthetic or natural compounds were closely related in structure to cilagicin.

### Cilagicin Antibacterial Activity and Resistance

When assayed more broadly, it was found that cilagicin was active against all Gram− positive pathogens that were tested with minimum inhibitory concentrations (MIC) ranging from 0.125 to 2 μg/mL (Table 4).

TABLE 4
Activity of cilagicin against microorganisms and human cells.

| | Cilagicin MIC |
|---|---|
| Pathogens/Human cells | (μg/mL)^{a} |
| Gram-positive | |
| Staphylococcus aureus USA300 (MRSA) | 1 |
| Staphylococcus aureus BAA1717(BRSA) | 0.5 |
| Enterococcus faecium EF18 (VRE) | 0.5 |
| Enterococcus faecalis AR785 (VRE) | 0.5 |
| Enterococcus gallinarum AR784 (VRE) | 0.5 |
| Enterococcus casseliflavus AR798 (VRE) | 0.125 |
| Streptococcus pneumoniae R^{b} | 0.5 |
| Streptococcus pneumoniae Tigr4^{b} | 0.25 |
| Clostridium difficile HM89^{c} | 2 |
| Clostridium difficile HM746^{c} | 2 |
| Streptococcus pyrogens ATCC19615 | 0.125 |
| Streptococcus agalactiae BAA2675 | 1 |
| Streptococcus agalactiae BAA1176 | 1 |
| Bacillus subtilis 168A1 | 2 |
| Gram-negative | |
| Acinetobacter baumannii ATCC17978 | 8 |
| Escherichia coli BAS849 | 4 |
| Escherichia coli ATCC25922 | >64 |
| Klebsiella pneumoniae ATCC13833 | >64 |
| Pseudomonas aeruginosa PAO1 | >64 |
| Enterobacter cloacae ATCC13047 | >64 |
| Human cell line | |
| HEK293 | >64^{c} |

^{a}The MIC was tested by broth microdilution.
^{b}Bacteria were cultured under 5% CO₂;
^{c}Bacteria were cultured under anaerobic condition; MRSA, methicillin-resistant S. aureus; BRSA, bacitracin-resistant S. aureus; VRE, vancomycin-resistant Enterococci.
^{c}IC₅₀

It was largely inactive against Gram-negative bacteria with the exception of A. baumannii (MIC 8 μg/mL) (Table 5) and outer membrane permeabilized E. coli BAS849 (MIC 4 μg/mL), indicating that the outer membrane of Gram-negative bacteria blocked cilagicin's access to its target.

TABLE 5
MIC of cilagicin against various A. baumannii strains.

| | | Resistant | Cilagicin MIC |
|---|---|---|---|
| Pathogen name | Strain | phenotype | (μg/mL) |
| Acinetobacter baumannii | 1775 | | 8 |
| Acinetobacter baumannii | 1788 | tetracycline | 8 |
| Acinetobacter baumannii | 1790 | tetracycline | 8 |
| Acinetobacter baumannii | 1791 | tetracycline | 8 |
| Acinetobacter baumannii | 1795 | tetracycline | 8 |
| Acinetobacter baumannii | 1797 | tetracycline | 8 |
| Acinetobacter baumannii | S3 | | 8 |
| Acinetobacter baumannii | S5 | | 8 |

In addition to its potent S. aureus activity, cilagicin exhibited potent activity against a number of difficult-to-treat vancomycin-resistant Enterococci pathogens as well as Clostridial difficile, which are considered urgent and serious threat pathogens by the CDC, receptively (CDC. Antibiotic Resistance Threats in the United States, 2019. Altlanta, GA: U.S. Department of Health and Human Services, CDC 2019). Cilagicin was particularly active against Streptococcus pathogens, including Streptococcus pneumoniae and Streptococcus pyrogens for which MICs were ≤0.25 μg/mL. Cilagicin was also active against all antibiotic resistant Gram-positive pathogens that were tested. For example, cilagicin maintained potent activity (MIC 1-2 μg/mL) against all members of a panel of 19 S. aureus strains that showed different patterns of resistance to clinically relevant families of antibiotics (Table 6).

TABLE 6
MIC of cilagicin against various antibiotic resistant S. aureus strains.

| | | | Cilagicin |
|---|---|---|---|
| | | Resistant | MIC |
| Pathogen name | Strain | phenotype | (ug/mL) |
| Staphylococcus aureus | USA 100 | M | 1 |
| Staphylococcus aureus | USA 200 | M | 1 |
| Staphylococcus aureus | USA 300 | M | 1 |
| Staphylococcus aureus | USA 500 | M | 2 |
| Staphylococcus aureus | USA 800 | M | 1 |
| Staphylococcus aureus | NRS 100 | T, O, M | 2 |
| Staphylococcus aureus | NRS 108 | G, O | 1 |
| Staphylococcus aureus | NRS 281 | E, S | 2 |
| Staphylococcus aureus | NRS 22 | V | 2 |
| Staphylococcus aureus | BAA-42 | M, O, P | 2 |
| Staphylococcus aureus | BAA-1721 | Hyper-virulent | 1 |
| Staphylococcus aureus | BAA-1556 | M, MP | 1 |
| Staphylococcus aureus | BAA-1717 | M, B | 0.5 |
| Staphylococcus aureus | NRS 146 | V | 1 |
| Staphylococcus aureus | NRS 140 | E, S | 1 |
| Staphylococcus aureus | COL (Anu 189) | | 2 |
| Staphylococcus aureus | HAR 22 | O | 2 |
| Staphylococcus aureus | HU 25 | O | 1 |
| Staphylococcus aureus | WCMC039967 | C, L | 2 |
| Staphylococcus aureus | WCMC069634 | C, L | 2 |
| Staphylococcus aureus | WCMC070502 | C, L | 2 |

M, methicillin;
T, tetracycline;
O, oxacillin;
G, gentamicin;
E, erythromycin;
S, spectinomycin;
V, vancomycin;
C, ciprofloxacin;
L, levofloxacin.

In stark contrast to FDA approved antibiotics, cilagicin also maintained potent activity (MIC≤1 μg/mL) against all strains found in a panel of 30 vancomycin-resistant Enterococci clinical isolates (Table 7).

TABLE 7
MIC of cilagicin against a panel of vancomycin-resistant Enterococci clinical isolates. MIC data to the right
of the dotted line was obtained from CDC&FDA antibiotic resistant isolate BANK at cdc.gov/ARIsolateBank/QA.

| | Enterococci | | | | | | | | |
|---|---|---|---|---|---|---|---|---|---|
| | | Van | | | | | | | |
| | Vancomycin | resistance | Cilagicin | Cilagicin | | | | | |
| | phenotype | MOA | phenotype | MIC | Vancomycin | Ampicillin | Chloramphenicol | Daptomycin | Doxycycline |
| Code | MIC (μg/mL) | | | | | | | | |
| 781 | R | A | S | 1 | >64 | 1 | 8 | 4 | 8 |
| 782 | R | B | S | 1 | 32 | 1 | >32 | 2 | ≤1 |
| 783 | R | B | S | 1 | 64 | 64 | 16 | 4 | 16 |
| 784 | R | C | S | 0.5 | 4 | 1 | 4 | 4 | ≤1 |
| 785 | S | | S | 0.5 | 2 | 2 | >32 | 2 | 8 |
| 786 | R | B | S | 0.125 | 32 | 0.5 | 16 | 0.5 | ≤1 |
| 787 | R | A | S | 1 | >64 | 8 | 32 | 8 | 32 |
| 788 | R | | S | 0.25 | 2 | 1 | 8 | 1 | ≤1 |
| 789 | R | A | S | 1 | >64 | 0.12 | 16 | 8 | ≤1 |
| 790 | R | B | S | 1 | >64 | 64 | 8 | 4 | ≤1 |
| 791 | S | | S | 0.5 | 0.5 | 1 | 8 | 1 | 4 |
| 792 | R | B | S | 1 | >64 | 64 | 4 | 4 | ≤1 |
| 793 | S | | S | 1 | 1 | 1 | 8 | 4 | ≤1 |
| 794 | R | A | S | 1 | >64 | 64 | 8 | 4 | ≤1 |
| 795 | R | A | S | 0.5 | 32 | 2 | 32 | 1 | 4 |
| 796 | R | C | S | 0.5-1 | 8 | 2 | 8 | 2 | 4 |
| 797 | R | | S | 0.125 | 2 | 1 | 8 | 1 | ≤1 |
| 798 | R | | S | 0.125 | 2 | 1 | 16 | 1 | ≤1 |
| 799 | R | B | S | 0.5 | >64 | 1 | >32 | 2 | 16 |
| 800 | R | B | S | 0.5 | 64 | 1 | 16 | 2 | ≤1 |
| 801 | R | | S | 0.25 | 4 | 1 | 8 | 2 | ≤1 |
| 802 | R | A | S | 1 | >64 | 64 | 8 | 4 | 16 |
| 803 | R | A | S | 1 | >64 | 128 | 8 | 4 | ≤1 |
| 804 | I | B | S | 0.5 | 16 | 1 | >32 | 1 | 16 |
| 805 | S | | S | 0.5 | 1 | 1 | 8 | 2 | ≤1 |
| 806 | S | | S | 1 | 0.5 | 4 | 8 | 4 | ≤1 |
| 807 | R | B | S | 1 | >64 | 32 | 32 | 4 | 4 |
| 808 | R | C | S | 0.25 | 4 | 2 | 8 | 4 | ≤1 |
| 809 | R | A | S | 0.5 | >64 | 0.5 | 8 | 4 | ≤1 |
| 810 | S | | S | 0.5 | 1 | 1 | 8 | 4 | 16 |

| | Enterococci | | | | | | | |
|---|---|---|---|---|---|---|---|---|
| | | | | | | Quinupristin/ | | |
| | Gentamicin | Streptomycin | Levofloxacin | Linezolid | Penicillin | dalfopristin | Rifampin | Teicoplanin |
| Code | MIC (μg/mL) | | | | | | | |
| 781 | ≤500 | ≤1000 | 2 | 2 | 2 | 4 | 1 | >64 |
| 782 | >500 | >1000 | 1 | 2 | 2 | 16 | 1 | 0.5 |
| 783 | >500 | >1000 | 4 | 2 | >32 | 0.5 | >4 | 2 |
| 784 | ≤500 | ≤1000 | 2 | 1 | 2 | 2 | ≤0.25 | 0.5 |
| 785 | >500 | >1000 | 1 | 2 | 4 | 16 | 2 | 0.5 |
| 786 | >500 | ≤1000 | 0.5 | 2 | 1 | 8 | 2 | 0.25 |
| 787 | ≤500 | >1000 | 2 | 2 | 32 | 16 | ≤0.25 | >64 |
| 788 | ≤500 | ≤1000 | 2 | 4 | 1 | 2 | >4 | 1 |
| 789 | >500 | >1000 | 4 | 2 | ≤0.12 | 1 | >4 | >64 |
| 790 | ≤500 | >1000 | >8 | 2 | >32 | 0.5 | >4 | 1 |
| 791 | ≤500 | ≤1000 | 1 | 2 | 2 | 1 | 1 | 0.5 |
| 792 | >500 | ≤1000 | >8 | 1 | >32 | 0.5 | 4 | 1 |
| 793 | ≤500 | ≤1000 | 1 | 2 | 2 | 4 | 4 | 0.5 |
| 794 | >500 | >1000 | >8 | 2 | >32 | 0.5 | 4 | 64 |
| 795 | >500 | >1000 | 0.5 | 1 | 4 | 16 | 2 | 0.5 |
| 796 | ≤500 | ≤1000 | 2 | 2 | 2 | 2 | >4 | 1 |
| 797 | ≤500 | ≤1000 | 2 | 2 | 0.5 | 2 | 4 | 1 |
| 798 | ≤500 | ≤1000 | 4 | 4 | 1 | 2 | ≤0.25 | 1 |
| 799 | >500 | >1000 | 1 | 2 | 2 | 16 | 2 | 0.5 |
| 800 | >500 | ≤1000 | 0.5 | 2 | 2 | 8 | 2 | 0.5 |
| 801 | >500 | ≤1000 | 2 | 2 | 1 | 4 | 0.5 | 1 |
| 802 | >500 | >1000 | 2 | 2 | >32 | 0.5 | ≤0.25 | 64 |
| 803 | >500 | >1000 | 4 | 4 | >32 | 1 | >4 | >64 |
| 804 | >500 | >1000 | 1 | 2 | 4 | 16 | 2 | 0.5 |
| 805 | ≤500 | ≤1000 | 1 | 2 | 2 | 4 | 2 | 0.5 |
| 806 | ≤500 | ≤1000 | 2 | 2 | 8 | 2 | >4 | 1 |
| 807 | ≤500 | >1000 | 8 | 2 | >32 | 0.5 | >4 | 1 |
| 808 | ≤500 | ≤1000 | 2 | 4 | 2 | 2 | >4 | 0.5 |
| 809 | ≤500 | >1000 | >8 | 2 | 1 | 16 | 2 | 64 |
| 810 | ≤500 | >1000 | 1 | 2 | 2 | 8 | 2 | 0.5 |

This collection was highly enriched in MDR isolates with more than half exhibiting resistance to between five and eight different clinically used antibiotics. In addition to no clinical isolates showing resistance to cilagicin, colonies that showed more than a 2-fold increase in MIC (2 μg/mL) when plating S. aureus directly on cilagicin-containing agar plates were never observed.

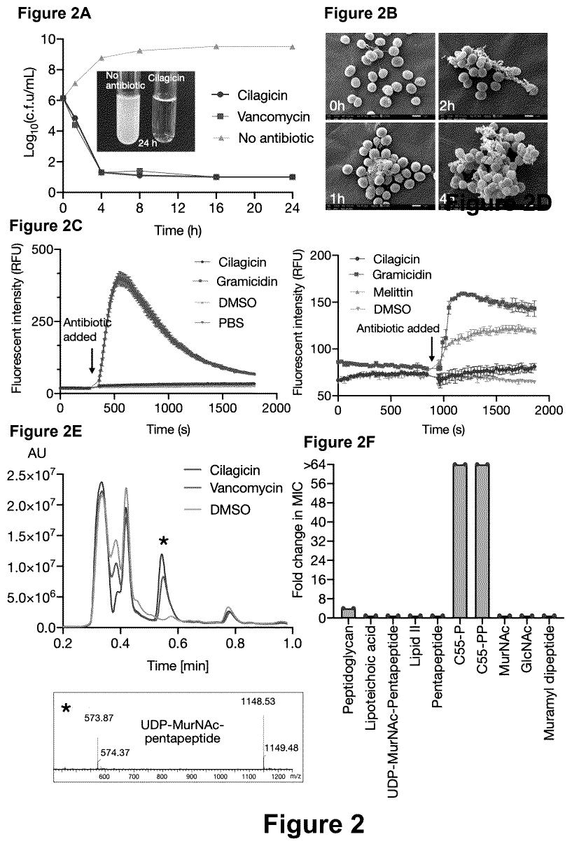

In a time-dependent killing curve analysis, cilagicin was found to be bactericidal and to reduce the number of viable bacteria by more than four orders of magnitude after 4 hours (FIG. 2A). Electron microscopy images of cilagicin treated cells showed cell collapse over time (FIG. 2B). At the highest concentration that was tested (64 μg/ml), cilagicin did not show human cell line (HEK293) cytotoxicity (Table 4). The absence of resistance together with its potent bactericidal activity and no human cell line toxicity led to explore cilagicin's mode of action in more detail.

### Mode of Action Studies

Potent antibacterial activity, with no detectable resistance, can point to a detergent-like activity (Bechinger et al., 2006, Biochim Biophys Acta, 1758:1529-1539; Shai et al., 1999, Biochim Biophys Acta, 1462:55-70). Cilagicin was therefore tested for membrane depolarization and cell lytic activities using 3,3′-dipropylthiadicarboncyanine iodide (DiSC3(5)) and SYTOX based fluorescence assays, respectively (FIG. 2C and FIG. 2D) (Roth et al., 1997, Appl Environ Microbiol, 63:2421-2431; Cabrini et al., 1986, J Membr Biol, 92:171-182; Wu et al., 1999, Biochemistry, 38:7235-7242). No response was detected in either assay when S. aureus was exposed to even 8-fold cilagicin's MIC, ruling out membrane disruption as its MOA.

Cilagicin is a zwitterion with two positively charged residues (3-D-Dab, 11-D-Dab) and two negatively charged residues (4-Asp, 7-D-Asp). Charged lipopeptide antibiotics often do not enter the cell and instead function by disrupting synthesis of the cell wall outside the cell membrane (Hover et al., 2018, Nat Microbiol, 3:415-422; Wu et al., 2019, J Am Chem Soc, 141:3910-3919; Cochrane et al., 2016, Proc Natl Acad Sci USA, 113:11561-11566).

Antibiotics that block peptidoglycan biosynthesis led to the accumulation of the lipid II precursor UDP-MurNAc-pentapeptide, which is easily detected by LCMS in antibiotic exposed cultures (Hover et al., 2018, Nat Microbiol, 3:415-422; Ling et al., 2015, Nature, 517:455-459; Schneider et al., 2009, Antimicrob Agents Chemother, 53:1610-1618; Kleijn et al., 2016, J Med Chem, 59:3569-3574). LCMS analysis of S. aureus cultures exposed to cilagicin (1×MIC) showed an obvious accumulation of UDP-MurNAc-pentapeptide (FIG. 2E).

As it is often much more difficult to alter a small molecule target than a protein target through genomic mutations, the inability to identify cilagicin resistant mutants hinted at the binding of a small molecule instead of a protein as the mode of inhibiting cell wall biosynthesis. To identify metabolite(s) that interact with cilagicin, a series of lipid II intermediates were screened for the ability to suppress cilagicin's antibacterial activity (FIG. 2F). In these studies, the MIC of cilagicin against S. aureus USA300 was determined in the presence of a 5-fold molar excess of each metabolite. Two of the compounds that were tested, undecaprenyl phosphate (C55-P) and undecaprenyl pyrophosphate (C55-PP), suppressed cilagicin's antibacterial activity.

C55-P is a monophosphorylated 55 carbon-long isoprene that is essential for transporting intermediates in the biosynthesis of cell wall carbohydrate polymers (e.g., peptidoglycan, O antigen, teichoic acids and others) across the bacteria cell membrane (Touz et al., 2008, EcoSal Plus, 3; van Heijenoort et al., 2001, Nat Prod Rep, 18:503-519). C55-PP is the di-phosphorylated version of the same 55 carbon isoprene. It is produced both de novo and recycled from C55-P during the biosynthesis of the cell wall. Its dephosphorylation by membrane-embedded pyrophosphatases generate the cellular pool of C55-P that is required for cell wall synthesis (Ghachi et al., 2018, Nat Commun, 9:1078).

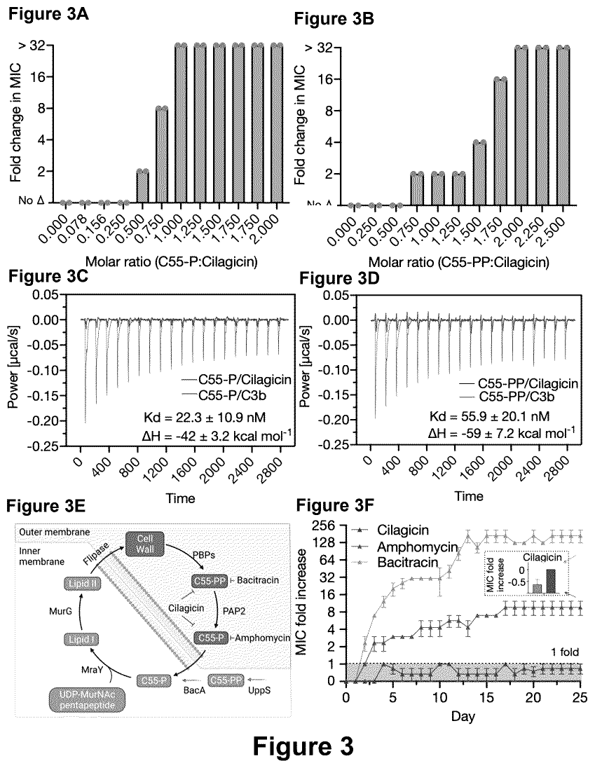

C55-P and C55-PP both show dose dependent inhibition of cilagicin's antibacterial activity (FIG. 3A and FIG. 3B). At a molar ratio of ~1.25, C55-P completely inhibited the bioactivity of cilagicin (MIC≥64 μg/mL). In the case of C55-PP, complete inhibition occurred at a molar ratio of ~2.5.

Using isothermal titration calorimetry (ITC), it was observed that cilagicin binds C55P and C55PP with measured dissociation constants (Kds) of 22 nM and 56 nM, respectively. In a similar ITC experiment using a representative inactive analog from the initial synthesis studies (cilagicin-3b) it was found that this closely related inactive structure did not bind either compound (FIG. 3C and FIG. 3D).

Collectively, the MOA studies are consistent with cilagicin being able to sequester both C55-P and C55-PP (i.e., bifunctional) and, in doing so, disrupt peptidoglycan biosynthesis. Moreover, cilagicin is the first antibiotic capable of sequestering the entire pool of phosphorylated undecaprenyl metabolites, thereby representing an unprecedented antibacterial MOA.

Bacteria only have a small pool of free undecaprenyl carrier lipids (~105 molecules per cell) to use in the transfer of critical biosynthetic intermediates across the cell membrane (Hernandez-Rocamora et al., 2018, Cell Surf, 2:1-13). Although disruption of this process is an appealing antibacterial MOA it remains underexploited clinically as only a few antibiotics have been identified that bind even one undecaprenyl phosphate. These include the antibiotics bacitracin and tripropeptin, which specifically bind C55-PP in zinc and calcium dependent manners, respectively (Hashizume et al., 2011, Antimicrob Agents Chemother, 55:3821-3828; Stone et al., 1971, Proc Natl Acad Sci USA, 68:3223-3227). The only known antibiotics that bind C55-P are the calcium dependent lipopeptide friulimicin and its close congeners (e.g., amphomycin, laspartomycin) (Schneider et al., 2009, Antimicrob Agents Chemother, 53:1610-1618; Kleijn et al., 2016, J Med Chem, 59:3569-3574; Singh et al., 2016, Sci Rep, 6:31757). Binding C55-P directly reduces the amount of available C55-P, while sequestering C55-PP indirectly reduces C55-P by preventing C55-PP dephosphorylation. In either case, this disrupts the flow of peptidoglycan precursors into the cell-wall, ultimately leading to cell death (FIG. 3E).

Bacitracin is used clinically as a topical antibiotic and friulimicin is in development for use in animal health. Unfortunately, bacteria exposed to antibiotics that bind a single undecaprenyl phosphate are reported to readily develop resistance.

Antibiotics with multiple molecular targets tend to have reduced rates of resistance because of the difficulty associated with altering multiple targets simultaneously. Although not bound by any particular theory, it was hypothesized that in the case of cilagicin, its ability to bind both undecaprenyl phosphorylates (i.e., two distinct small molecules) would lead to a reduced resistance rate compared to antibiotics that bind a single phosphorylated undecaprenyl moiety.

As the studies failed to identify mutants resistant to cilagicin by direct plating on antibiotic containing media, S. aureus resistant mutants were raised by daily serial passage in the presence of sub-MIC levels of antibiotic. This was done using cilagicin, bacitracin or the friulimicin congener amphomycin to allow a direct comparison of resistance rates for antibiotics that bind either one or two phosphorylated undecaprenyl moieties. S. aureus rapidly developed resistance to both bacitracin and amphomycin.

MICs for these antibiotics increased by 8- to 256-fold during the course of the serial passage experiment. In contrast, after 25 days of constant exposure the maximum change in MIC, there was a 1-fold increase for cilagicin (FIG. 3F). In addition, neither the highly bacitracin resistant mutants nor the highly amphomycin resistant mutants that were generated showed cross resistance to cilagicin. Interestingly, the cil BGC is found in the genome of P. mucilaginosus. P. mucilaginosus 's negative Gram staining indicates the presence of an outer membrane that would protect it from cilagicin's toxicity thereby eliminating the need for self-resistance elements to have evolved in nature (Hu et al., 2010, Int J Syst Evol Microbiol, 60:8-14; Goswami et al., 2015, Cogent Food & Agriculture, 1:1000714).

### Animal Study

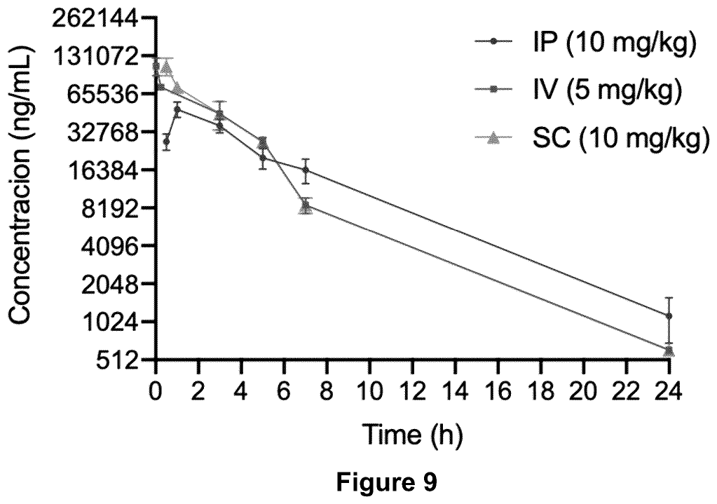

The initial pharmacological assessment in mice showed cilagicin had high plasma bioavailability when delivered by intraperitoneal (IP) injection (FIG. 9). However, it did not reduce bacterial burden in an animal infection model. It was subsequently observed that cilagicin's antibacterial activity was significantly suppressed in the presence of serum suggesting that high serum binding might have also limited its in vivo activity (FIG. 4A). Subsequent studies therefore attempted to generate a cilagicin analog with reduced serum binding. To do this a collection of cilagicin analogs as generated with different N-terminal lipids and compared their MICs in the presence and absence of serum. An analog containing a biphenyl N-terminal substituent (cilagicin BP) maintained good antimicrobial activity and showed no increase in MIC in the presence of serum (FIG. 4B, Table 8).

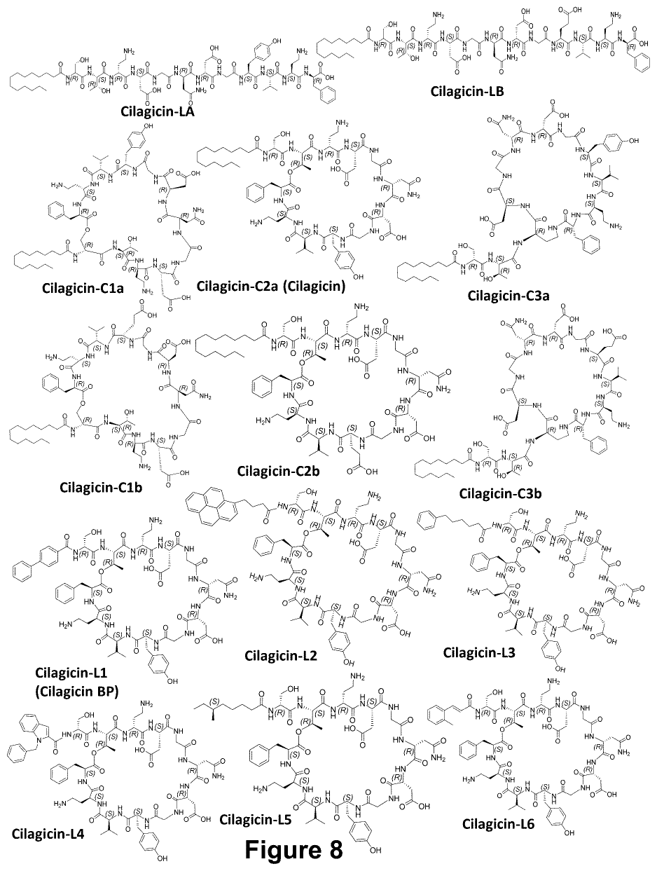

TABLE 8
Antimicrobial spectrum of cilagicin analogs against
a panel of Gram-positive pathogens. Structures of analogs
L1, L2, L3, L4, L5 and L6 are shown in FIG. 8.

| | MIC μg/mL | | | | | | |
|---|---|---|---|---|---|---|---|
| Pathogen | Cilagicin | L1(BP) | L2 | L3 | L4 | L5 | L6 |
| S. aureus USA300 | 1 | 4 | 0.5 | 8 | 8 | 4 | 64 |
| S. aureus USA300 + | 32 | 4 | 8 | 8 | 16 | 4 | 64 |
| 10% Serum | | | | | | | |
| E. faecium Com15 | 0.5 | >64 | 4-8 | 64 | 64 | 64 | >64 |
| S. pneumoniae Tigr4 | 0.25 | 4 | 1 | 4 | 8 | 4 | 32 |
| S. pyrogens ATCC | 0.125 | 1 | 0.125 | 1 | 1 | 1 | 4 |
| 19615 | | | | | | | |
| S. agalactiae | 1 | 16 | 2 | 16 | 16 | 8 | 64 |
| ATCC2675 | | | | | | | |
| C. difficile HM89 | 2 | 16 | 2 | 16 | 16 | 16 | >64 |

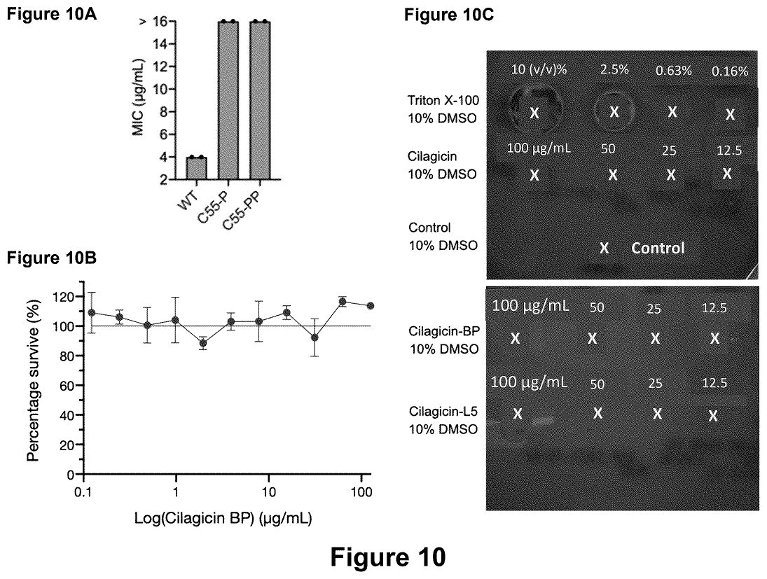

This change of lipid substituent did not alter its MOA (FIG. 10A), and cilagicin BP continued to show no hemolytic activity and no human cell cytotoxicity (FIG. 10 and FIG. 3C). Studies also assessed the in vivo efficacy of cilagicin BP using a neutropenic mouse thigh model. At 24 hours post infection, cilagicin BP showed significant antibacterial activity against S. aureus USA300 at 40 mg/kg (thrice daily, TID), resulting in an almost 4 log₁₀ reduction in colony forming units (CFU) compared to the vehicle control (FIG. 4C).

Next, cilagicin BP was evaluated against S. pyrogens ATCC19615 in the same neutropenic thigh model. In this case, cilagicin BP showed an even more impressive reduction (>5 log₁₀) in bacterial burden compared to the vehicle control, which was consistent with the lower MIC values for this pathogen in vitro (FIG. 4D). Against S. pyrogens, cilagicin BP resulted in more than a log greater reduction of bacterial burden than vancomycin.

### CONCLUSION

Infections caused by MDR pathogens are a serious and growing risk to healthcare systems around the world. The chemical diversity among naturally occurring lipopeptides has provided a number of privileged scaffolds that have proved useful in developing clinically relevant antibiotics. Numerous uncharacterized BGC that encode additional lipopeptides have been identified in bacterial genome sequencing data. In this study, Cs phylogeny was used to classify cryptic sequenced lipopeptide biosynthetic gene cluster families on a genetic level. This analysis guided the identification of the cil BGC, which in turn inspired the synthesis of the antibiotic cilagicin.

Cilagicin uniquely sequesters both C55-P and C55-PP, which significantly reduces frequency of resistance compared to antibiotics that bind individual phosphorylated undecaprenyl moieties. In fact, studies have so far failed to identify any cilagicin resistance in the laboratory or among clinical isolates. The suppression of cilagicin's antibacterial activity by serum was dramatically reduced with the synthesis of a biphenyl analog, cilagicin BP. Cilagicin BP's attractive and unique mode of action, absence of any detectable resistance and in vivo activity make it an appealing lead structure for the development of a next generation antibiotic that is capable of helping to address the growing antibiotic resistance crisis.

In summary, the lack of new therapeutics to address the growing antibiotic resistance crisis poses a threat to global public health. As most of the biosynthetic capacity within the bacterial kingdom has remained silent in previous antibiotic discovery efforts, uncharacterized biosynthetic gene clusters found in bacterial genome sequencing studies remain an appealing potential source of new antibiotics. The present studies report the discovery of the naturally inspired lipopeptide antibiotic “cilagicin” that was chemically synthesized based on a detailed bioinformatic analysis of the uncharacterized cil biosynthetic gene cluster. Cilagicin's unique ability to sequester two distinct indispensable undecaprenyl phosphates used in cell wall biosynthesis, together with the absence of detectable resistance in laboratory tests and among multi-drug resistant clinical isolates, makes it an appealing candidate for combating antibiotic resistant pathogens.

The materials and methods employed in the present experimental examples are now described.

### Identification and Bioinformatic Analysis of Cilagicin Biosynthetic Gene Cluster (BGC)

A total of 36,957 non-ribosomal peptide synthetase (NRPS) BGCs were retrieved from antismash-db (v2.0) using a parallel job execution tool “GNU parallel” from antiSMASH (v5.1.2, bacterial version). An in-house Perl Script (gist.github.com/yzhemand/e6d1779eb3b81957d4ab11c9cf085982) using a Bio::SeqIO module from BioPerl was written to identify lipopeptide BGCs by detecting the presence of a condensation starter (Cs) domain in these BGCs. In this analysis, the Perl Script read GenBank files one at a time and collected species name, number of A-domains, Cs domain sequence, and the whole cluster sequence for each BGC. Only BGCs containing a Cs domain and 5 or more A domains were used in the analysis. Cs domains were extracted from these BGCs and aligned using the MUSCLE algorithm in MacVector 18.0. Phylogenetic trees were visualized using iTOL v6 online software. For the cilagicin BGC, the 10 amino acids that makes up each A-domain binding pocket (i.e., amino acids 235, 236, 239, 278, 299, 301, 322, 330, 331, and 517) were identified using antiSMASH (v5.1.2). To determine the substrate of each cil BGC A-domain, A-domain substrate specificity signature sequences from the cil BGC were compared to a curated list of A-domain substrate specificity sequences from functionally characterized A-domains.

### Solid Phase Peptide Synthesis

Cilagicin and its analogs were synthesized using standard Fmoc-based solid-phase peptide synthesis (SPPS) methods on 2-chlorotrityl chloride resin. Cilagicin-C1a, C1b, C2b, C3a, and lipid analogs of cilagicin were synthesized starting from the glycine at the₅th position of the peptide. 2-cholorotrityl resin pre-loaded with glycine (0.3 g, 0.45 mmol/g) was swollen in DCM for 30 minutes at room temperature then drained and washed with DMF (3 mL, 3×). Subsequent couplings were carried out using Fmoc-protected amino acids (or a lipid) (2 equiv. relative to resin loading) mixed with HATU (2 equiv.) and DIPEA (2 equiv.) in DMF (3 mL). Each coupling reaction was carried out for 1 hour then washed with DMF (3 mL, 3×). Fmoc deprotection was carried out by treating resin-bound peptide with 20% piperidine in DMF (3 mL) for 7.5 minutes. This was repeated twice and the resin was then washed with DMF (4 mL, 5×). The synthesis of cilagicin-La, Lb, C2a, and C3b began with 2-cholorotrityl resin pre-loaded with phenylalanine (0.3 g, 0.486 mmol/g). Coupling and deprotection were done as described above.

Ester bondformation: Ester bonds were formed between the serine (cilagicin-C1a, C2b) or threonine (cilagicin-C1b, C3a) hydroxyl and the carboxylic acid of the C-terminal amino acid (phenylalanine) as follows. The resin-bound peptide with a free hydroxyl group was mixed with Fmoc-Phe (20 equiv.), DIPEA (40 equiv.), benzoyl chloride (20 equiv.) and DMAP (0.8 equiv.) in 15 mL DCM and gently shaken for 72 hours. After ester bond formation, the remaining amino acids were coupled as described above to form linear peptides.

Alloc group removal: Cilagicin-C2a and C3b contained Alloc protected diaminobutyric acids (Dab) at position 3 whereas all other cilagicin analogs contained Boc-protected Dab. This was done to allow differential deprotection of the Dab and thereby cyclization of cilagicin-C2a and C3b through the amine on Dab and the carboxylic acid at the C-terminus. Alloc deprotection was carried out as follows. Resin-bound linear peptide was washed with DCM (4 mL, 5×) and treated with phenylsilane (15 equiv.) and tetrakis(triphenylphosphine) palladium (0) (0.5 equiv.) in DCM at room temperature in an argon environment. After 2 hours, the resin was washed with 10% w/v sodium diethyldithiocarbamate trihydrate (50 mL DMF) and DCM (5 mL, 10×).

Peptide cyclization: Resin-bound linear peptides (C1a, C1b, C2a, C2b, C3a, C3b, and lipid analogs) were cleaved by treating with 25% hexafluroisopropanol in DCM for 1 hour. They were then collected by filtration and air-dried. The cleaved linear peptides were cyclized without purification by mixing with PyAOP (8 equiv.) and DIPEA (30 equiv.) dissolved in DMF (100 mL). After 2 hours, DCM (200 mL) was added and washed repeatedly with 1% formic acid in water (10 mL, 10×). The extracted peptides were air-dried overnight.

Final cleavage: Peptides were dissolved in 3 mL of cleavage cocktail (95% (v/v) TFA, 2.5% (v/v) triisopropylsilane and 2.5% (v/v) water) for 1.5 hours. A cold mixture of diethyl ether:hexane (1:1) was then added and kept at −20° C. for 20 minutes to precipitate the peptide. Peptide pellets were harvested by centrifuging (2500 g) for 5 minutes, re-dissolved in 10 mL methanol and dried in a speed-vac overnight.

Peptide purification: Crude peptides were purified on an Xbridge Prep C18 HPLC column (Waters) using a dual solvent system (A/B: water/acetonitrile, supplemented with 0.1% (v/v) formic acid). Peptide purity and identity were confirmed by UPLC, HRMS, and NMR. Pure peptides were dissolved in DMSO at 6.4 mg/mL for biological assays.

### Minimum Inhibitory Concentration (MIC) Assay

MIC assay were conducted using the protocol recommended by the Clinical and Laboratory Standards Institute.(37) Culture conditions (temperature, medium) are detailed in Table 3. All compounds were dissolved in sterile DMSO (ATCC, USA) to give a concentration of 6.4 mg/mL. Vancomycin hydrochloride was used as positive controls. Tested compounds were serially diluted across 96-well plates using a 2-fold serial dilution in a volume of 50 μL. An overnight culture of an assay strain was diluted 5,000-fold in fresh medium. 50 μL of this dilution was added into each well, giving a final volume of 100 μL in each well. Compounds were assayed at concentrations ranging from 64 μg/mL to 0.06 μg/mL. The top and bottom rows of each plate were filled with 100 μL of media to avoid edge effects. MIC values were recorded as the minimum concentration at which no bacterial growth appeared, based on visual inspection, after 16 hours of static incubation at 37° C. For Clostridium difficile plates were statically incubated under anaerobic conditions (Vinyl anaerobic chamber, 37° C., 5% H₂, 5% CO₂, 90% N₂). For Streptococcus pneumoniae plates were statically incubated under aerobic conditions supplemented with 5% C02 (37° C.). All MICs were performed in technical duplicate (n=2) and repeated three independent times (n=3).

### Cytotoxicity Assay

The cytotoxicity of cilagicin and its analogs was tested using an MTT (3-(4,5-Dimethyl-2-thiazolyl)-2,5-diphenyl-2H-tetrazolium bromide) assay as previously described (Wikler et al., 2006, CLSI (NCCLS), 26:M7-A7). HEK293 cells were seeded in a 96-well plate with a density of 5,000 cells/well and cultured in Dulbecco's Modified Eagle Medium (DMEM) supplemented with 10% fetal bovine serum, 1% Pen/Strep and 1% glutamate for 24 hours at 37° C. with 5% CO₂. Serially diluted compounds were added into each well at a final concentration ranging from 64 μg/mL to 0.25 μg/mL. After 48 hours of incubation, the media was removed and 110 μL of freshly prepared MTT solution (0.45 mg/mL in DPBS) was added to each well. The plates were incubated for 3 hours at 37° C. with 5% C02 after which the MTT solution was removed by aspiration. Precipitated formazan crystals were dissolved by addition of 100 μL of solubilization solution (40% DMF, 16% SDS and 2% acetic acid in H₂O). The absorbance of each well was measured at OD₅₇₀ₙₘ using a microplate reader (Infinite 200 PRO, Tecan). Paclitaxel (Sigma, USA) and 0.25% DMSO were used as positive and negative controls, respectively. IC50 values were calculated using Prism 9.0 as the concentration of each compound required for 50% inhibition of cell growth compared to the negative control (0.25% DMSO, 100% growth) and positive control (4 μg/mL Paclitaxel, 0% growth). All the experiments were performed in triplicates (n=3) and repeated two independent times (n=2).

### Cell Lysis Assay

Cell lytic activity of cilagicin was evaluated using SYTOX green nucleic acid stain (Thermo Fisher, USA). Gramicidin and DMSO were used as positive and negative controls, respectively. S. aureus USA300 was grown overnight at 37° C. with shaking (200 rpm) and then diluted in fresh LB broth to an OD600ₙₘ of 0.35. 900 μL of this S. aureus USA300 suspension was mixed with 100 μL of SYTOX (17 μM in DMSO). The mixture was incubated at room temperature for 5 minutes, and then transferred into a 384-well flat bottom black microtiter plate (30 μL/well). The initial fluorescence intensity of each well was recorded using an infinite 200 PRO (Excitation/Emission=488/523 nm) at 9 second intervals for 5 minutes. 30 μL of 8×MIC solution of each test compound (cilagicin 8 μg/mL, gramicidin 32 μg/mL) was added into each well. The fluorescence intensity of each well was then continually monitored at 9 second intervals for 25 minutes and plotted by Prism 9.0. All assays were performed in biological triplicates (n=3).

### Membrane Depolarization Assay

The effect of cilagicin on membrane depolarization was analyzed using DiSC3(5) dye (Thermo Fisher, USA). Overnight cultures of S. aureus USA300 were harvested and resuspended in PBS (OD600 nm=0.37). Cell suspension (1 mL) and 10 μL of 4 μM DisC3(5) were pre-mixed and incubated in the dark at room temperature for 15 minutes. 50 μL of this mixture was then added to each well of black 96-well flat bottom black microtiter plates. The initial fluorescence intensity of each well was recorded using an infinite 200 PRO (Excitation/Emission=620/670 nm) at 9 second intervals for 15 minutes. 50 μL of a 1 μM solution of each test compound was then added to each well. Gramicidin (30 μM) and melittin (1 μM) were used as positive controls. DMSO was used as the vehicle control. The fluorescence intensity of each well was then monitored at 9 second intervals for another 15 minutes and plotted by Prism 9.0. All assays were performed in biological triplicate (n=3).

### Time-Dependent Killing Assay

An overnight culture of S. aureus USA300 was diluted 1:10,000 in 50 mL fresh LB and incubated at 37° C. with aeration at 220 rpm for 2 hours. Bacteria (3 mL) were challenged with 10× the MIC of each antibiotic (10 μg/mL cilagicin, 10 μg/mL vancomycin). After 1.25, 4, 8, 16 and 24 hours 200 μL aliquots of cells were collected, centrifuged at 10,000×g for 1 minute and then resuspended in 200 μL of sterile PBS (pH 7.4). 50 μL aliquots of 10-fold serial dilutions were plated on LB agar plates. After overnight growth at 37° C., colonies were counted. Experiments were performed three independent times (n=3).

### UDP-MurNAc-Pentapeptide Accumulation Assay

The effect of cilagicin on cell wall biosynthesis was investigated by tracking the accumulation UDP-MurNAc-pentapeptide in antibiotic treated cultures. Overnight S. aureus USA300 cultures were diluted in fresh LB broth and grown at 37° C. to an OD600 nm of 0.5. They were then treated with 130 μg/mL of chloramphenicol. After incubating at 37° C. for 15 minutes, 1 μg/mL of cilagicin was added. Vancomycin (10 μg/mL) and DMSO were used as positive and vehicle control, respectively. After an incubation of 60 mins, cells (0.5 mL) were harvested, and the cell pellet was resuspended in 30 μL ddH₂O. The cell suspension was incubated in boiling water for 15 minutes and spun down at 15,000×g for 5 minutes. The supernatant was then analyzed by UPLC-DAD-MS (Acquity UPLC BEH C18, 2.1×50 mm, 1.7 μM, 130 Å, 0.45 mE/min isocratic elution at 97% water:acetonitrile (ACN) for 1 minute, then from 97% to 5% water:ACN over 3 minutes, with constant 0.1% formic acid; negative ionization modes).

### Scanning Electron Microscopy

S. aureus USA300 cultures at different time points from the time-kill curve analysis (FIG. 2A, 0, 1, 2, and 4 hours) were used in this study. 30 μl of each cell suspension was spotted onto 12 mm glass or Aclar film round coverslips. A drop of fixative (100 μl of 2% glutaraldehyde, 4% formaldehyde in 0.1 M sodium cacodylate buffer pH 7.2) was added on top of the coverslip which was coated with 0.1% polylysine. After 30 minutes, additional fixative was added to the dish containing the coverslips and samples were left at 4° C. overnight. Samples were gently washed three times with 0.1 M sodium cacodylate buffer (pH 7.2) for 5-10 minutes each time and postfixed with osmium tetroxide 1% in 0.1M sodium cacodylate buffer pH 7.2 for 1 hour at room temperature. After rinsing three times with buffer, samples were dehydrated in a graded series of ethanol concentrations (30%, 50%, 70%, 90%) for 10 minutes each and three times in 100% ethanol (with molecular sieves) for 15 minutes each and then further dried in a critical point drier (Tousimis Autosamdri 931, USA). Samples were coated with 10 nm of iridium using a Leica ACE600 sputter coater.

### Imaging was Done in a JEOL JSM-IT500HR at 5.0 kV.

Isothermal titration calorimetry (ITC) The interaction of cilagicin and undecaprenyl phosphate (C55-P, Larodan, SE) or undecaprenyl diphosphate (C55-PP, Larodan, SE) was measured using ITC. Briefly, 0.1 μm of large unilamellar vesicles (LUVs) was prepared by mixing 1,2-dioleoyl-sn-glycero-3-phosphocholine (DOPC, Avanti, AL) and 0.1% of either C55-P or C55-PP in chloroform/methanol (2:1). The resulting lipid suspension was dried under argon gas for 2 hours and then rehydrated in 10 mM HEPES buffer (pH 7.5, 100 mM NaCl). The dried sample was filtered 10 times through an Avanti mini extruder (Avanti, AL) using a 0.1 μM filter membrane. ITC binding experiments were performed using a MicroCal Auto-iTC200. A vesicle suspension of 0.27 μM C55-P or 0.25 μM C55-PP and 2.5 mM DOPC in HEPES buffer was titrated into a freshly made solution of 0.1 mM cilagicin in the same buffer. The titration was conducted under the following conditions: temperature 25° C., reference power 5 uCals-1, syringe-stirring speed 750 rpm, number of injections 19, injection volume 2 μl, and time between injections 150 s. The calorimetric data obtained from the Auto-iTC200 instrument was analyzed using AFFINImeter software. Thermodynamic parameters (enthalpy (AH), entropy (AS), and the equilibrium binding constant (KD)) were calculated using a one-binding site model.

### Intermediate Feeding Assay

The effect of lipid II biosynthesis intermediates on cilagicin's antibacterial activity was evaluated using S. aureus USA300 and the general MIC assay methods described above with the following changes. Potential antagonizing compounds were mixed with cilagicin at a constant molar ratio of 5. Cilagicin mixed with solvent alone was used as a control. Premixed solutions were dried in vacuo for 2 hours to completely remove all organic solvent. They were then resuspended in LB broth and mixed with S. aureus. All assays were run in duplicate (n=2) and repeated two independent times (n=2).

### Resistance Studies

A single colony of S. aureus USA300 was inoculated in 5 mL LB broth and grown overnight at 37° C. with continuous shaking (200 rpm). The overnight culture was then diluted 1:5,000 into fresh LB. 50 μL aliquots of dilute cells were transferred into individual wells of 96-well plates containing 50 μL of serially diluted cilagicin, amphomycin (AG Scientific, USA) or bacitracin (Sigma, USA). Plates were statically incubated at 37° C. After 24 hours, the MIC was recorded. For the next round of assays, an aliquot from the culture with the second highest antibiotic concentration that showed cloudy growth was diluted 1:5,000 time in fresh LB and mixed with serial diluted antibiotics. The MIC was determined as described above. This process was repeated daily for 25 days. For bacitracin, the LB medium was supplemented with 50 μg/mL ZnCl₂. For amphomycin, the medium was supplemented with 100 μg/mL CaCl₂-2H₂O. Experiments were performed three independent times (n=3).

### In-Vivo Pharmacokinetics Studies

All animal studies were ethically reviewed and carried out in accordance with the Institutional Animal Care and Use Committee of Hackensack Meridian Health under protocol number of 269.01. Six-week-old CD-1 female mice (20-25 g) were used in pharmacokinetic studies. Cilagicin was administered as a single dose by intravenous (IV, 5 mg/kg), intraperitoneal (IP, 10 mg/kg), and subcutaneous (SC, 10 mg/kg) injection. Aliquots of 20 μL of blood were taken by puncture of the lateral tail vein from each mouse (n=3 per route and dose) at each timepoint. Blood timepoints were taken after dosing at 1 minute, 15 minutes, 1 hour, 3 hours, 7 hours, and 24 hours for IV dosing, and 30 minutes, 1 hour, 3 hours, 5 hours, 7 hours, and 24 hours for SC, IP, and PO. Blood was captured in CB300 blood collection tubes containing K2EDTA and stored on ice. Plasma was recovered after centrifugation (3,500×g, 5 minutes) and stored at −80° C. until analyzed by HPLC-MS/MS. Cilagicin was formulated in 5% DMSO and 95% water, which resulted in a clear solution.

### HPLC-MS/MS (High Pressure Liquid Chromatography Coupled Tandem Mass Spectrometry) Pharmacokinetic Analysis

Cilagicin (1 mg/mL, DMSO) was serial diluted in ACN:water (50:50) to create standard curves and quality control (QC) spiking solutions. Standards and QCs were created by adding 10 μLs of spiking solutions to 90 μL of drug free plasma (CD-1 K2EDTA Mouse, Bioreclamation IVT). Cilagicin was extracted by combining 10 μL of plasma with 100 μL ACN:methanol (MeOH) (50:50) (precipitation solvent) containing 10 ng/mL of the internal standard (IS) verapamil (Sigma Aldrich, USA). Extracts were vortexed for 5 minutes and centrifuged at 3200×g for 5 minutes. 75 μL of supernatant was used for HPLC-MS/MS analysis. HPLC-MS/MS analysis was performed on a Sciex Applied Biosystems Qtrap 6500+ triple-quadrupole mass spectrometer coupled to a Shimadzu Nexera X2 UHPLC (Ultra-High-Performance Liquid Chromatography) system to quantify each drug in the plasma. Chromatography was performed on a Phenomenex Luna Omega Polar C18 column (2.1×100 mm; particle size, 3 μm) using a reversed phase gradient. Milli-Q deionized water with 0.1% formic acid was used for the aqueous mobile phase and ACN with 0.10% formic acid was used for the organic mobile phase. Multiple-reaction monitoring (MRM) of parent/daughter transitions in electrospray positive-ionization mode was used to quantify the analytes. The double charged ion was used for cilagicin. The following MRM transitions were used for cilagicin (733.96/72.00) and Verapamil (455.40/165.00). Sample analysis was accepted if the concentrations of the quality control samples were within 20% of the nominal concentration. Data processing was performed using Analyst software (v1.6.2; Applied Biosystems Sciex).

### Hemolytic Assay

The hemolytic activity of cilagicin was evaluated using a red blood cell disc diffusion assay. Cilagicin and other test compounds were dissolved in 10% DMSO to give concentrations ranging from 100 μg/mL to 12.5 μg/mL. 10% (v/v) Triton X-100 and 10% DMSO were used as positive and vehicle controls, respectively. 20 μL of each serially diluted compound was infused on a sterile disc. Infused discs were overlaid on the surface of a 5% sheep blood agar plate (Hardy diagnostics, USA) and incubated at 20° C. for 24 hours. The size of the transparent ring that appeared in the agar plate was measured to determine hemolytic activity. Experiments were performed two independent times. A representative picture was taken of one experiment.

### Neutropenic Thigh Infection Model

All procedures in the animal study were ethically reviewed and carried out in accordance with the IACUC (Institutional Animal Care and Use Committees) at the Rockefeller University under protocol 19032-H. Six-week-old female outbred Swiss Webster mice (20-25 g, Charles River, USA) were used for this experiment. Mice were randomly housed in individually ventilated cages (IVC). The room was set on a twelve-hour light cycle and the temperature was set to 70° F. The humidity was set to 30%. To initiate the neutropenic model, mice were injected with 150 mg/kg and 100 mg/kg of cyclophosphamide via intraperitoneal (IP) injection on days −4 and −1, respectively. On day −1, a single colony of S. aureus USA300 was grown in 15 mL of cation adjusted Mueller Hinton (MH) broth at 37° C. with shaking overnight. On day 0, cells were centrifuged, washed twice in 0.9% sterile saline, and resuspended in sterile 0.9% saline solution to give final optical density (600 nm) of 0.1. 50 μL of bacteria suspension was injected into both thighs via intramuscular (IM) injection. This provides a challenge inoculum of approximately 1.0×10⁶ CFU in each thigh in a volume of 50 μL. At 2 hours post infection, mice were given 200 μL of vehicle (10% DMSO), vancomycin (40 mg/kg, 10% DMSO) or cilagicin BP (40 mg/kg, 10% DMSO) at 2, 10, and 18 hours post infection via IP injection. Mice in the pretreatment group (n=4 mice/n=8 thighs) were humanely euthanized by CO₂ narcosis to determine the starting thigh bacterial burden. 24 hours post the infection, mice were humanely euthanized by CO₂ narcosis (n=4 mice/n=8 thighs for each condition). Thigh muscles were aseptically removed, weighed, homogenized and enumerated for bacterial burden by CFU counts after plating on MH agar. All graphic data are expressed as data points by group and were statistically analyzed using Prism software (Prism 9). For S. pyrogens ATCC19615, LBM broth and agar medium detailed in Table 3 were used to culture and plate the bacteria.

What is claimed is:

1. A compound comprising the amino acid sequence D(X^{A})ₐK,
wherein each occurrence of X^{A} is independently selected from the group consisting of a natural amino acid, functionalized natural amino acid, unnatural amino acid, functionalized unnatural amino acid, and any combination thereof; and
wherein a is an integer from 8 to 100.

2. The compound of claim 1, wherein the compound is a zwitterion.

3. The compound of claim 2, wherein the zwitterion comprises at least two positively charged residues and at least two negatively charged residues.

4. The compound of claim 1, wherein the compound is a cyclic compound.

5. The compound of claim 1, wherein the compound is a linear compound.

6. The compound of claim 1, wherein the amino acid sequence D(X^{A})ₐK comprises at least one amino acid sequence selected from the group consisting of at least one amino acid sequence as set forth in SEQ ID NOs: 1-12 or a fragment thereof.

7. The compound of claim 1, wherein a is an integer represented by 8.

8. The compound of claim 1, wherein the functionalized natural amino acid or the functionalized unnatural amino acid comprises a functional group selected from the group consisting of at least one selected from FIG. 4A, at least one selected from FIG. 6, at least one selected from FIG. 7, and any combination thereof.

9. The compound of claim 1, wherein the compound is a compound having the structure of Formula (I) 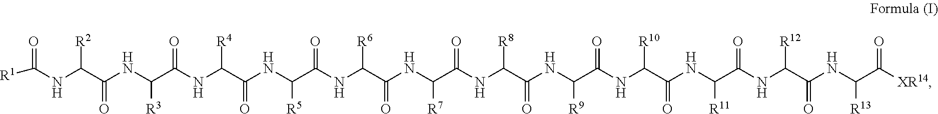
or a racemate, an enantiomer, a diastereomer, a pharmaceutically acceptable salt, or a derivative thereof;
wherein X is selected from the group consisting of O, S, and N(R¹⁴); and
each occurrence of R¹, R², R³, R⁴, R⁵, R⁶, R⁷, R⁸, R⁹, R¹⁰, R¹¹, R¹², R¹³, and R¹⁴ is independently selected from the group consisting of hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, and any combinations thereof;
wherein R² and X are optionally fused or joined to form a ring;
R³ and X are optionally fused or joined to form a ring; or
R⁴ and X are optionally fused or joined to form a ring.

10. The compound of claim 9, wherein the compound having the structure of Formula (I) is a compound having the structure selected from the group consisting of 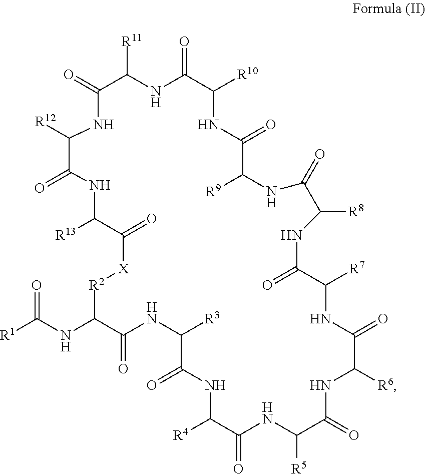
or a racemate, an enantiomer, a diastereomer, a pharmaceutically acceptable salt, or a derivative thereof; 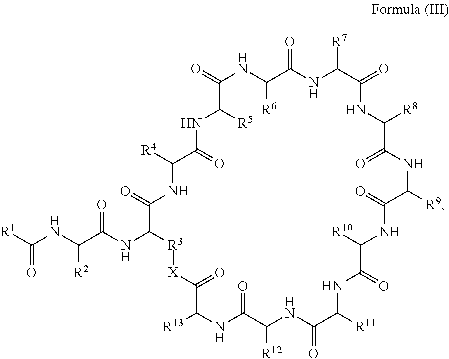
or a racemate, an enantiomer, a diastereomer, a pharmaceutically acceptable salt, or a derivative thereof; and 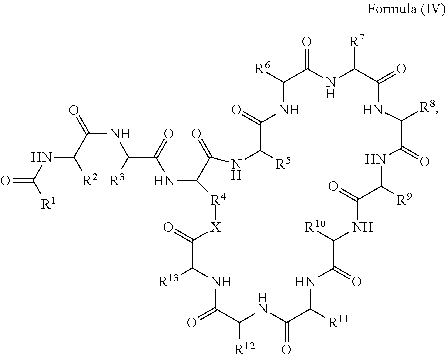
or a racemate, an enantiomer, a diastereomer, a pharmaceutically acceptable salt, or a derivative thereof;
wherein X is selected from the group consisting of O, S, and N(R¹⁴); and
each occurrence of R¹, R², R³, R⁴, R⁵, R⁶, R⁷, R⁸, R⁹, R¹⁰, R¹¹, R¹², R¹³, and R¹⁴ is independently selected from the group consisting of hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, and any combinations thereof.

11. The compound of claim 1, wherein the compound is a compound selected from the group consisting of 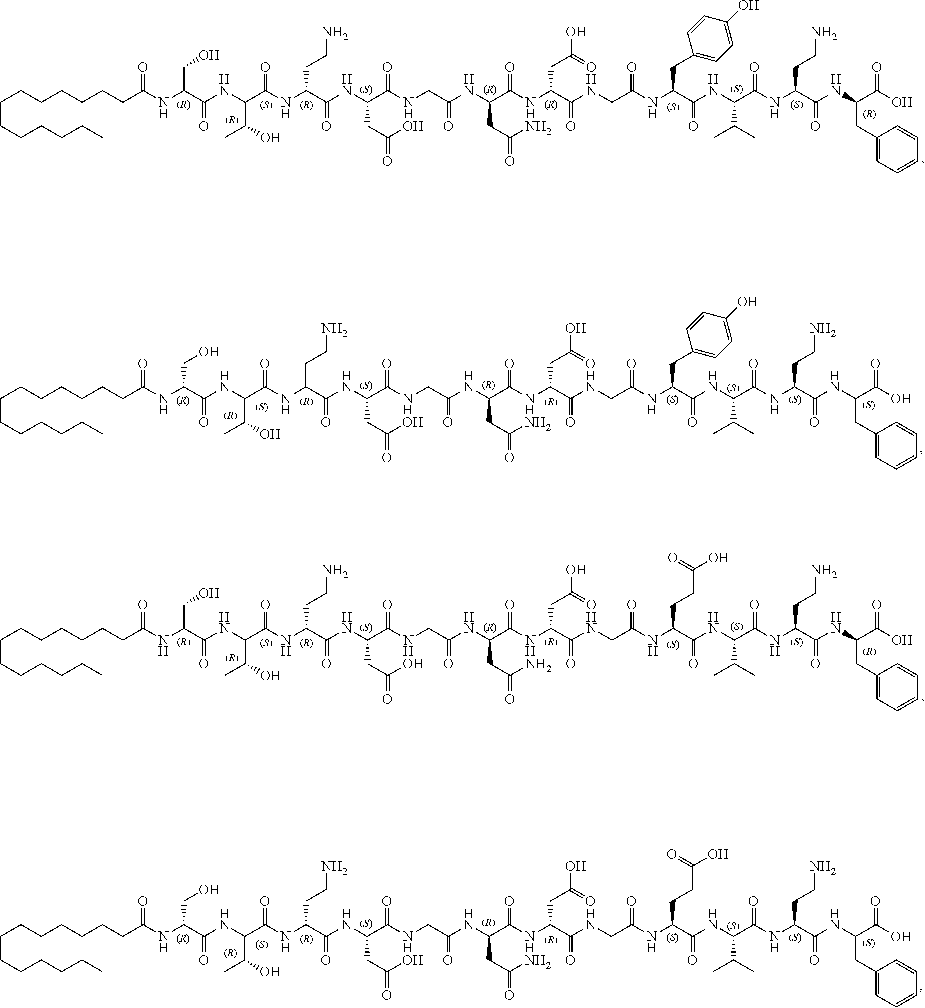 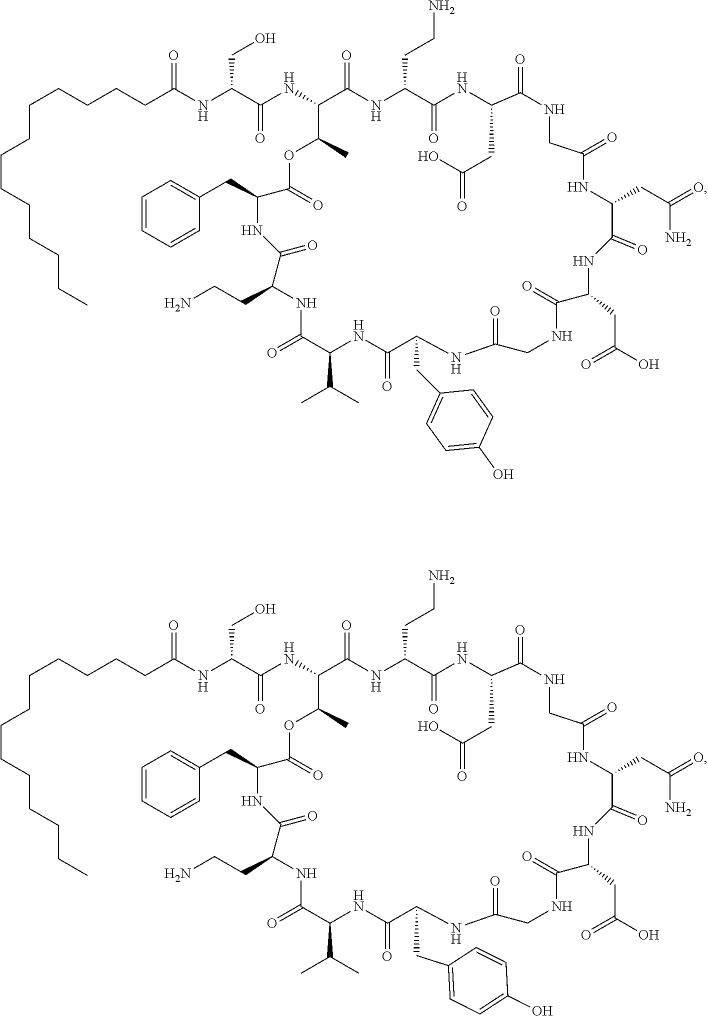 [SMILES: CCCCCCCCCCCCCC(=O)N[C@H](CO)C(=O)N[C@@H]1C(=O)N[C@H](CCN)C(=O)N[C@@H](CC(=O)O)C(=O)NCC(=O)N[C@H](CC(N)=O)C(=O)N[C@H](CC(=O)O)C(=O)NCC(=O)N[C@@H](Cc2ccc(O)cc2)C(=O)N[C@@H](C(C)C)C(=O)N[C@@H](CCN)C(=O)N[C@@H](Cc2ccccc2)C(=O)O[C@@H]1C ; CCCCCCCCCCCCCC(=O)N[C@H](CO)C(=O)N[C@@H]1C(=O)N[C@H](CCN)C(=O)N[C@@H](CC(=O)O)C(=O)NCC(=O)N[C@H](CC(N)=O)C(=O)N[C@H](CC(=O)O)C(=O)NCC(=O)N[C@@H](Cc2ccc(O)cc2)C(=O)N[C@@H](C(C)C)C(=O)N[C@@H](CCN)C(=O)N[C@@H](Cc2ccccc2)C(=O)O[C@@H]1C] 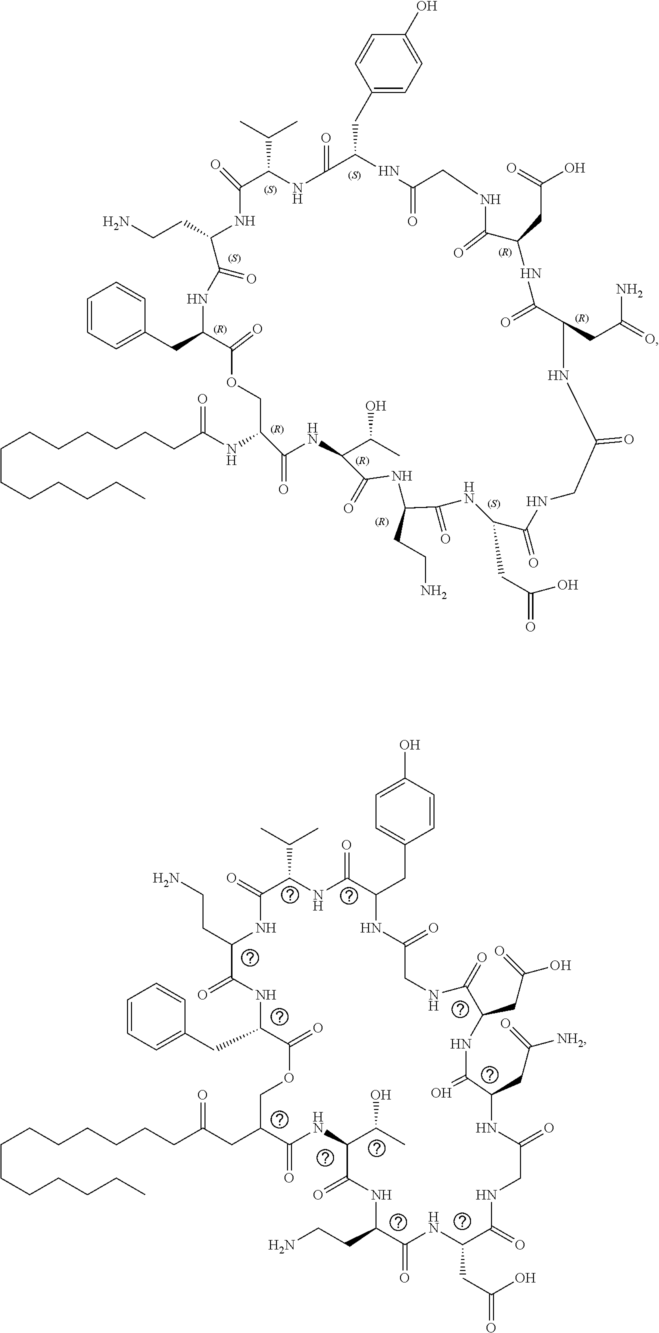 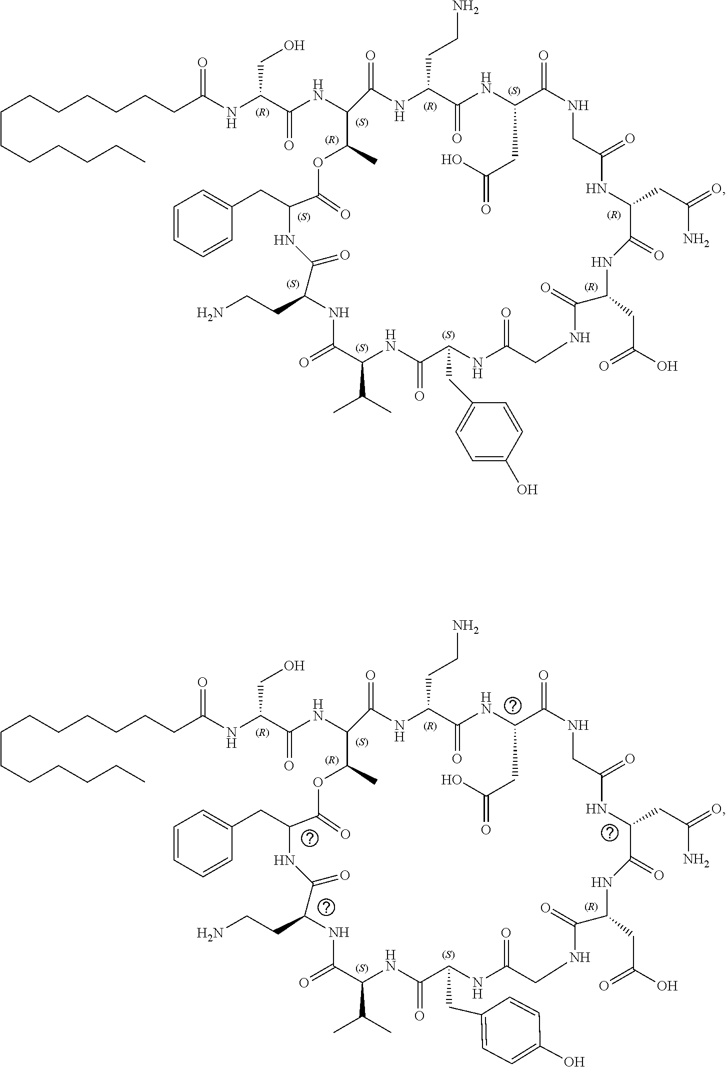 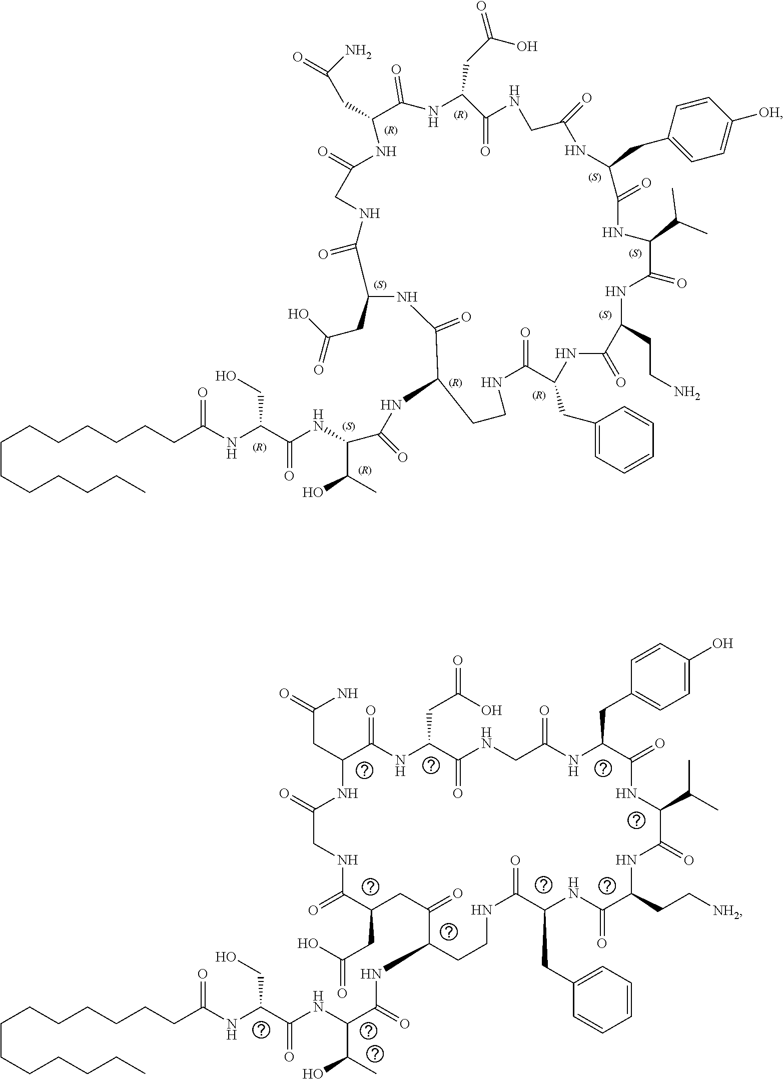 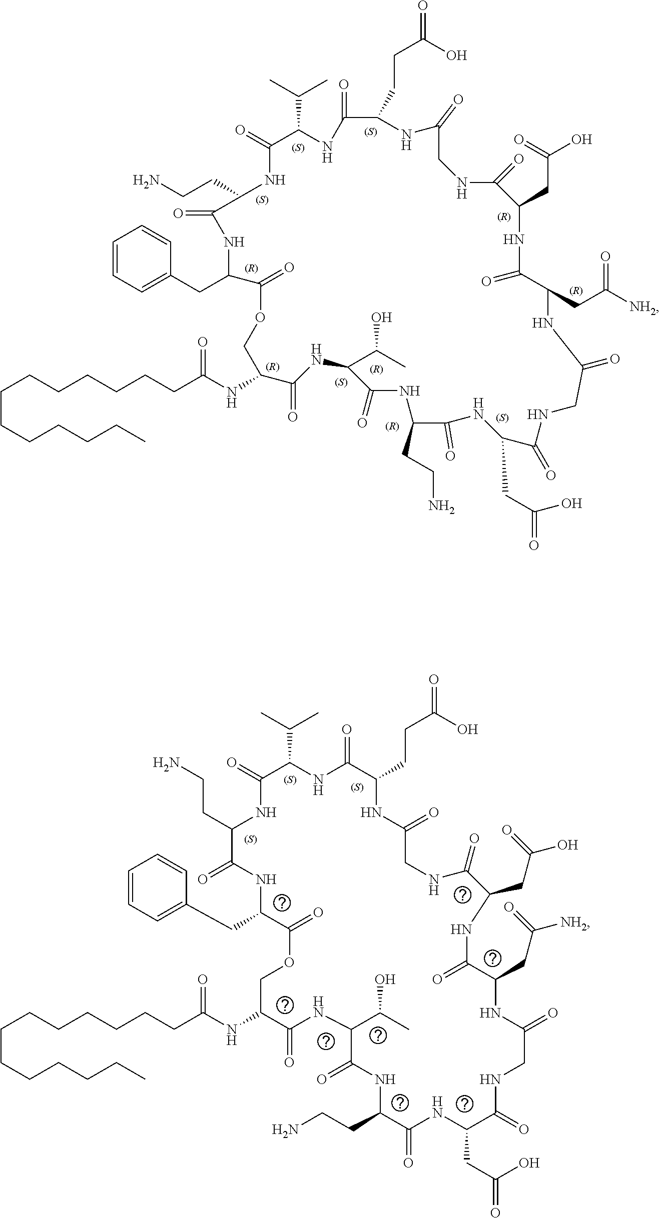 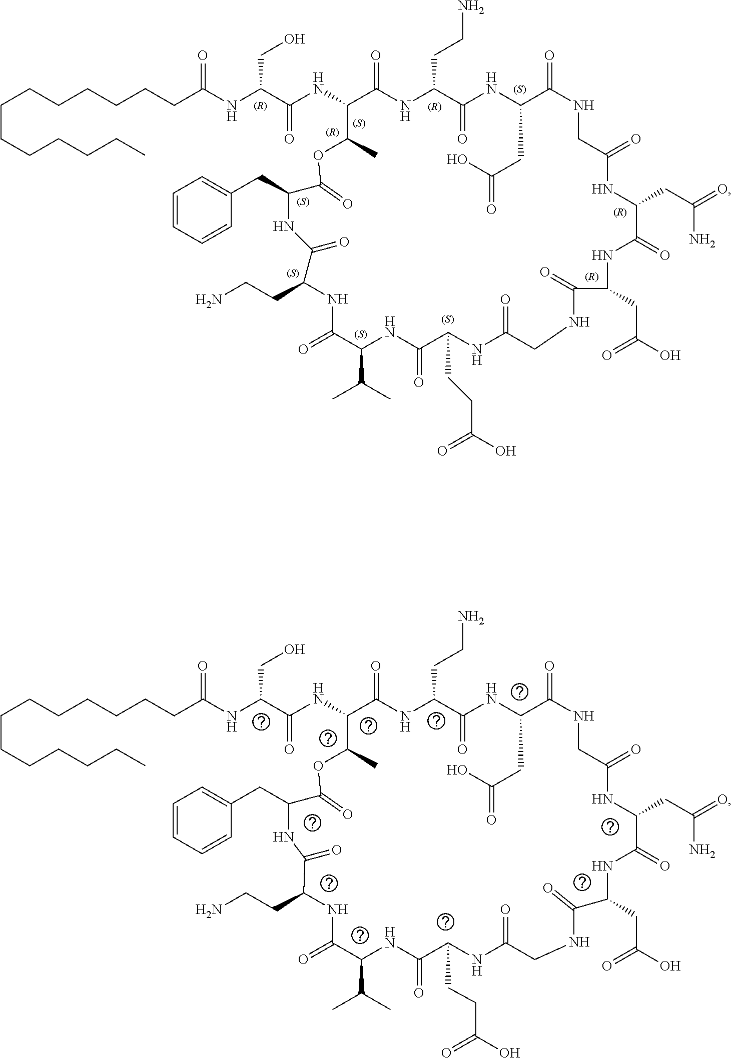 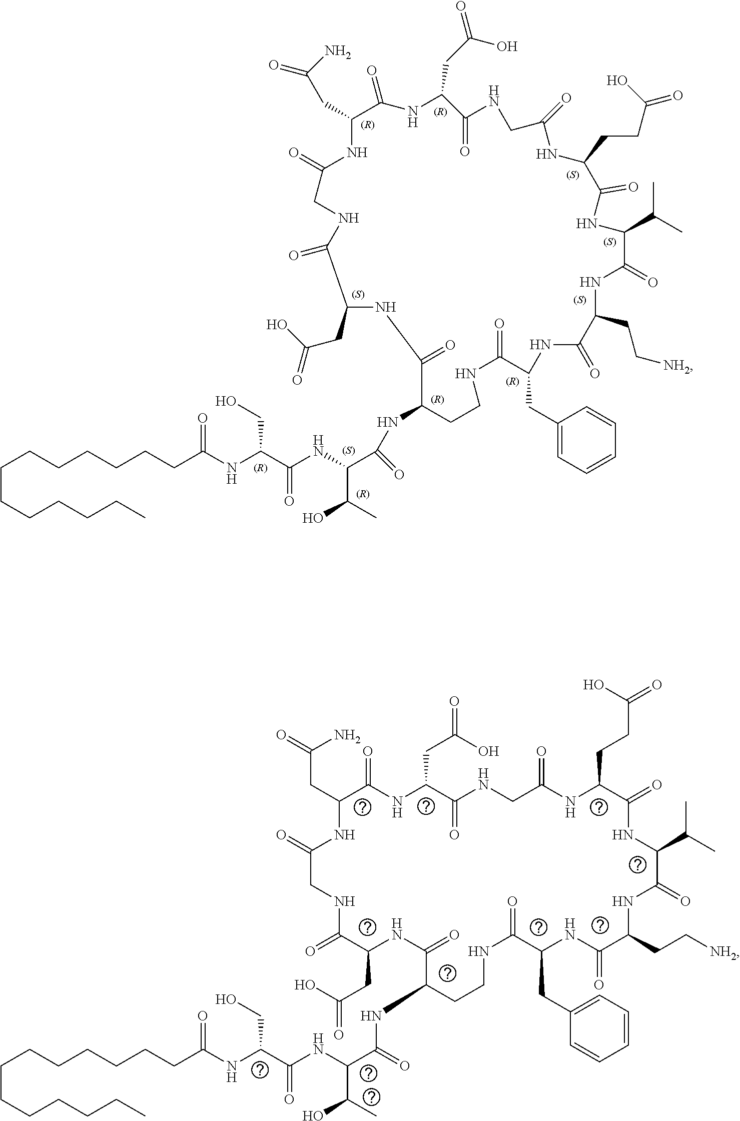 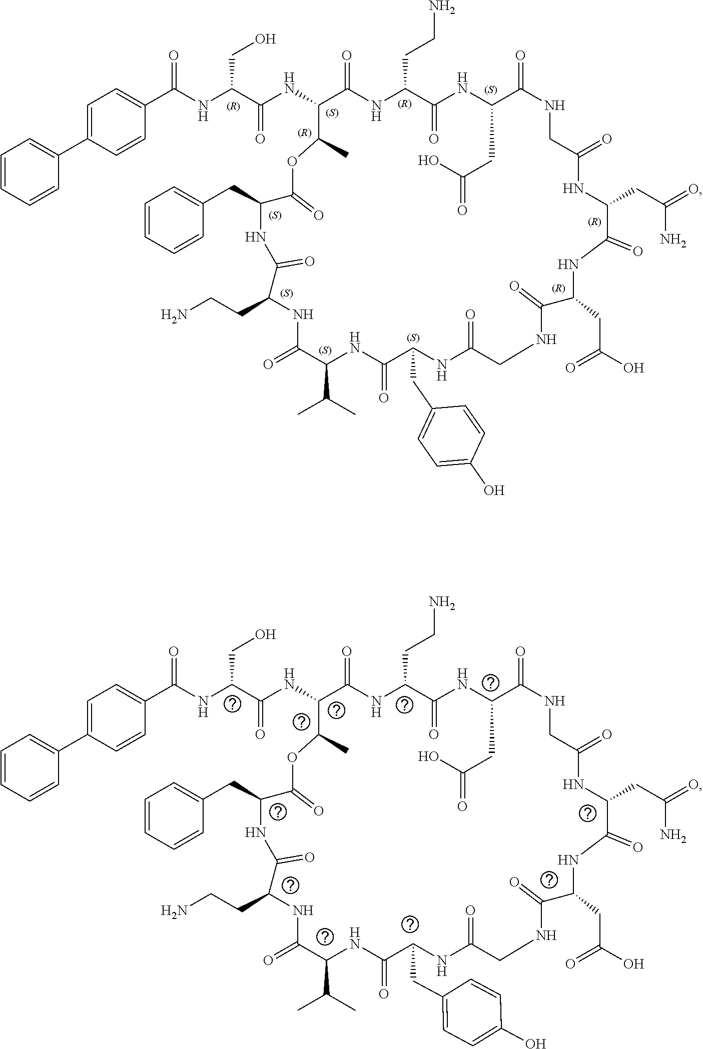 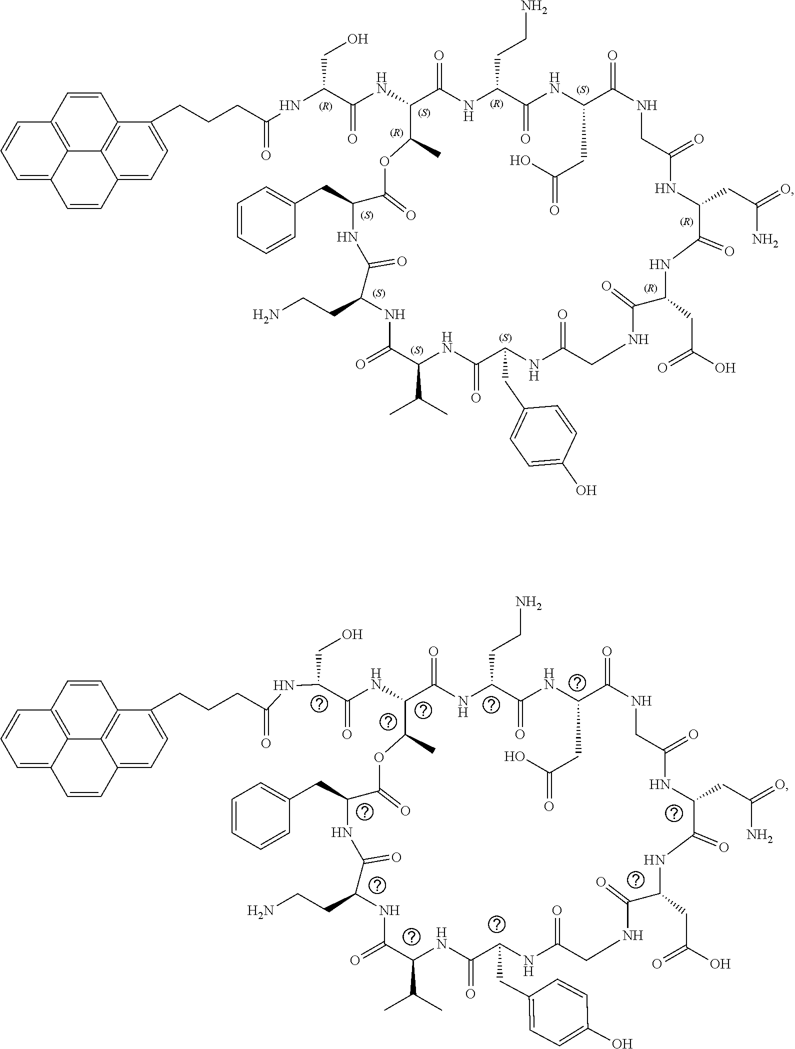 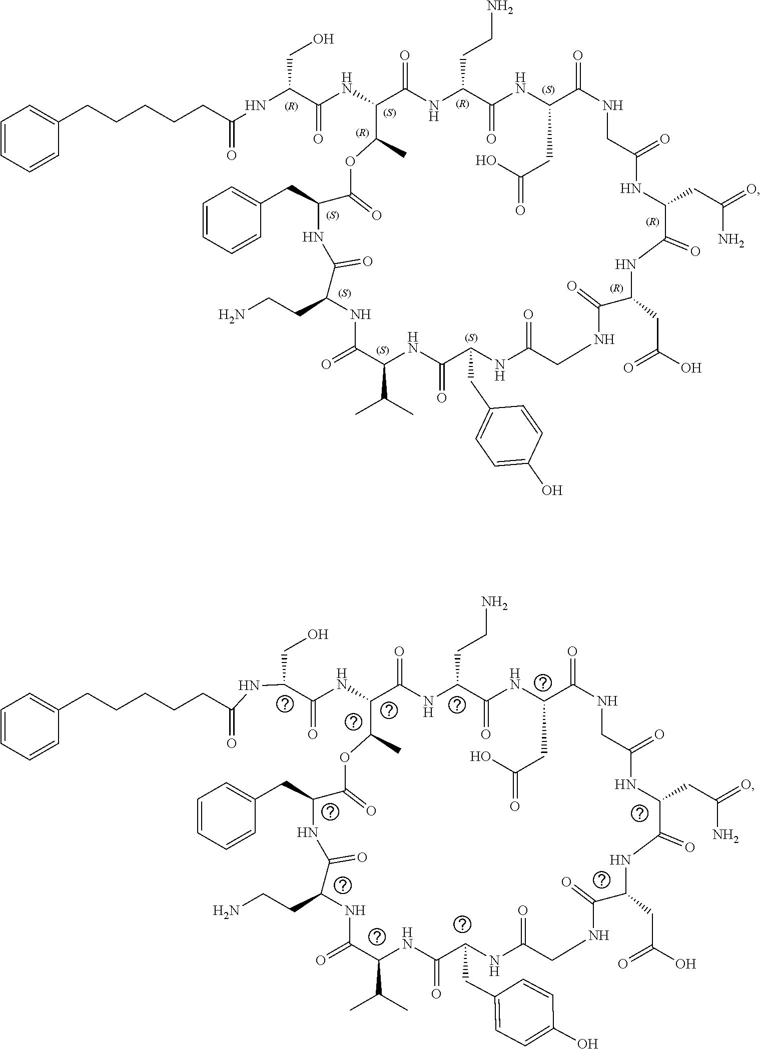 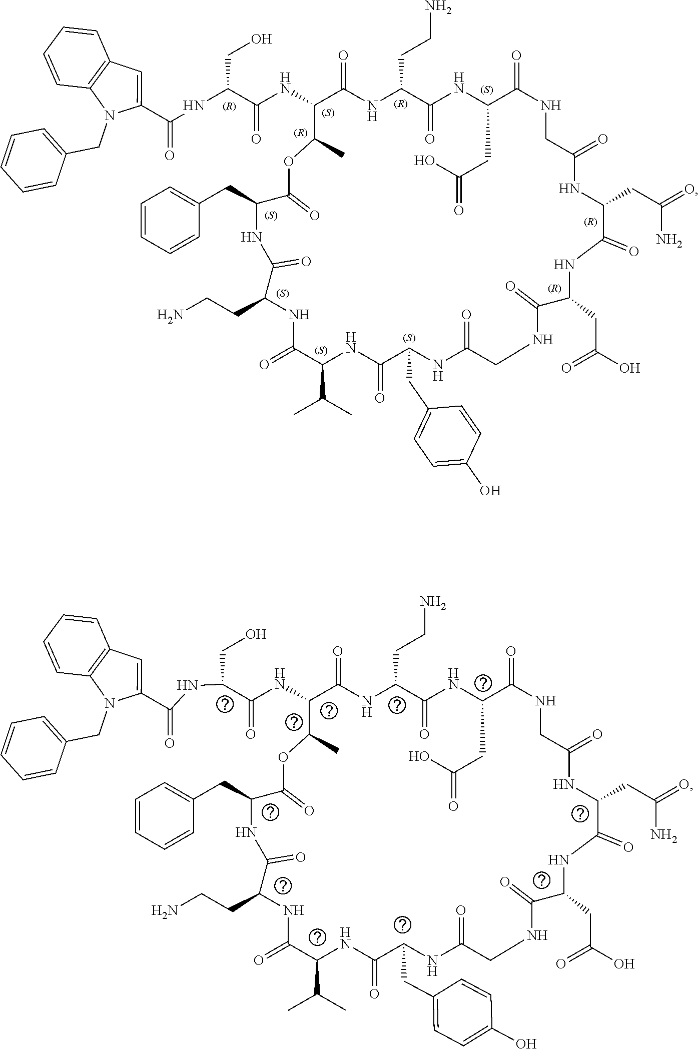 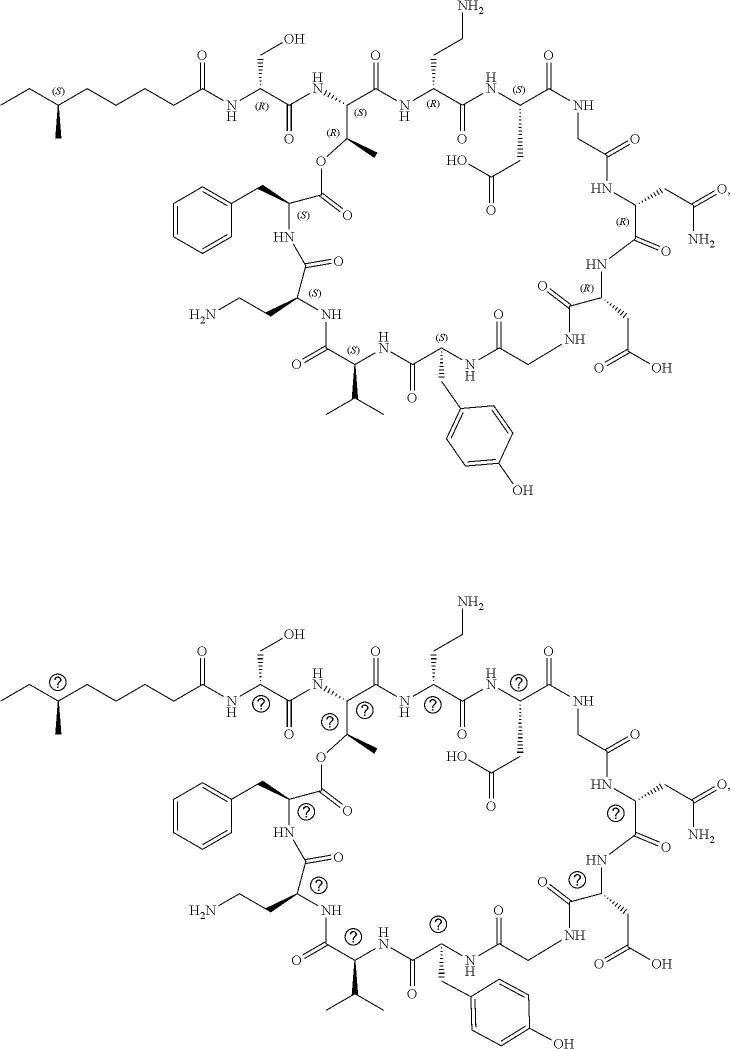 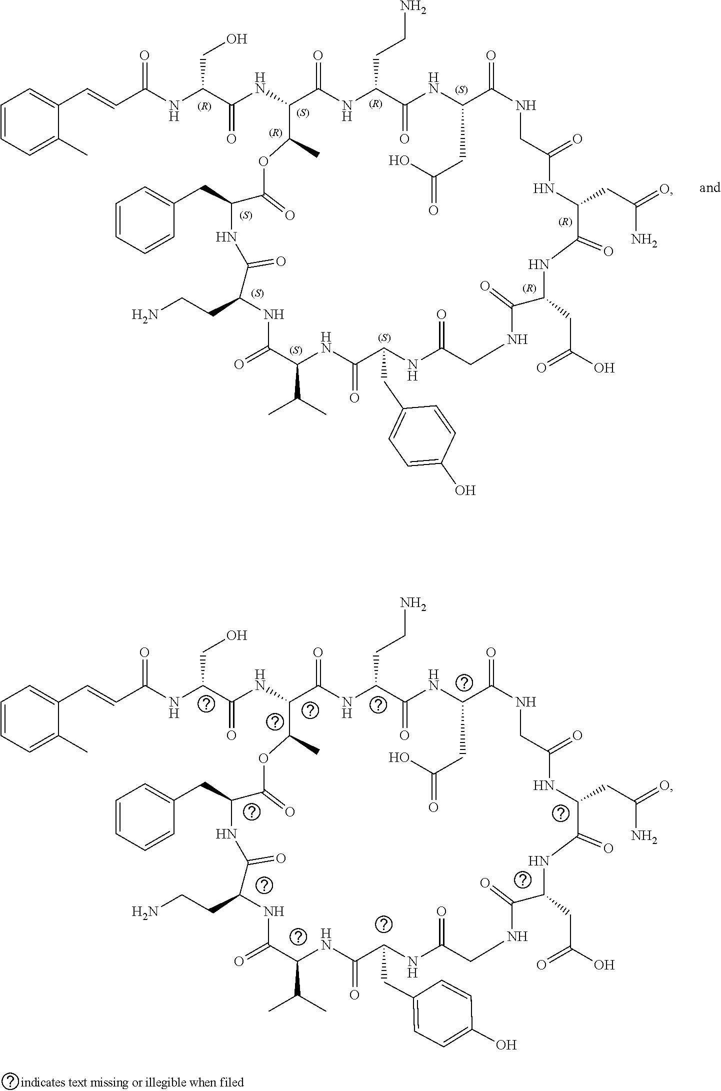
or a racemate, an enantiomer, a diastereomer, a pharmaceutically acceptable salt, or a derivative thereof.

12. The compound of claim 1, wherein the compound inhibits cell wall biosynthesis.

13. The compound of claim 1, wherein the compound specifically binds at least one undecaprenyl phosphorylate.

14. A pharmaceutical composition comprising at least one compound of claim 1 or a racemate, an enantiomer, a diastereomer, a pharmaceutically acceptable salt, or a derivative thereof.

15. An isolated nucleic acid molecule encoding at least one compound of claim 1 or a racemate, an enantiomer, a diastereomer, a pharmaceutically acceptable salt, or a derivative thereof.

16. The isolated nucleic acid molecule of claim 15, wherein the nucleic acid molecule comprises at least one nucleotide sequence of FIG. 1.

17. A genetically engineered cell producing at least one compound of claim 1 or a racemate, an enantiomer, a diastereomer, a pharmaceutically acceptable salt, or a derivative thereof.

18. A method of treating or preventing a bacterial infection in a subject in need thereof, the method comprising administering at least one compound of claim 1 or a composition thereof to the subject.

19. The method of claim 18, wherein the subject is exposed to or infected with a pathogen.

20. The method of claim 19, wherein the pathogen is bacteria.

21. The method of claim 20, wherein the bacteria is selected from the group consisting of drug resistant bacteria, gram positive bacteria, and any combination thereof.

22. The method of claim 20, wherein the bacteria is selected from the group consisting of Bacillus subtilis, Clostridium difficile, Enterococcus faecium, Enterococcus gallinarum, Enterococcus casseliflavus, Escherichia coli, Streptococcus agalactiae, Streptococcus pneumoniae, Streptococcus pyrogens, Staphylococcus aureus, Klebsiella pneumoniae, Acinetobacter baumannii, Pseudomonas aeruginosa, Enterobacter cloacae, Enterobacter species, and any combination thereof.

23. The method of claim 18, wherein the method further comprises administering a second therapeutic.

24. The method of claim 23, wherein the second therapeutic is an antibiotic.

25. A method of inhibiting the growth of or killing a bacterial cell, the method comprising, contacting the bacterial cell with at least one compound of claim 1 or a composition thereof.

26. A method of biosynthesizing at least one compound of claim 1, the method comprising:
a) providing a nucleic acid to a host or a growth medium, wherein the nucleic acid encodes the amino acid sequence D(X^{A})ₐK or a fragment thereof,
wherein each occurrence of X^{A} is independently selected from the group consisting of a natural amino acid, functionalized natural amino acid, unnatural amino acid, functionalized unnatural amino acid, and any combination thereof; and
wherein a is an integer from 8 to 100;
b) incubating the host in a growth medium; and
c) isolating the compound from the host or the growth medium.
